(19) United States
(12) Patent Application Publication
(10) Pub. No.: US 2024/0293532 A1
(43) Pub. Date: Sep. 5, 2024

(54) REPLICATION-COMPETENT ADENOVIRUS TYPE 4 SARS-COV-2 VACCINES AND THEIR USE

(71) Applicant: The U.S.A., as Represented by The Secretary, Department of Health and Human Services, Bethesda, MD (US)

(72) Inventor: Mark Connors, Bethesda, MD (US)

(73) Assignee: The U.S.A., as Represented by The Secretary, Department of Health and Human Services, Bethesda, MD (US)

(21) Appl. No.: 18/271,901

(22) PCT Filed: Jan. 14, 2022
(86) PCT No.: PCT/US2022/012530

§ 371 Date: Jul. 12, 2023

Related U.S. Application Data
(60) Provisional application No. 63/138,221, filed on Jan. 15, 2021.

(51) Int. Cl. A61K 39/215; A61K 9/00; A61K 39/00; A61P 31/14; C12N 7/00

(52) U.S. Cl. CPC A61K 39/215; A61K 9/0043; A61P 31/14; C12N 7/00; A61K 2039/5254; A61K 2039/543; A61K 2039/545; C12N 2750/14121; C12N 2750/14122; C12N 2750/14134

(57) ABSTRACT

A replication-competent adenovirus type 4 (Ad4) modified to express the SARS-COV-2 spike protein is described. The genome of the recombinant Ad4 is modified to have a deletion of at least a portion of the adenovirus E3 region to accommodate insertion of the spike protein coding sequence. Administration of the recombinant Ad4 to the upper respiratory tract elicits mucosal immunity, which is important for protection against SARS-COV-2 infection and for preventing transmission of the virus.

### CROSS REFERENCE TO RELATED APPLICATIONS

This application claims the benefit of U.S. Provisional Application No. 63/138,221, filed Jan. 15, 2021, which is herein incorporated by reference in its entirety.

### FIELD

This disclosure concerns a recombinant replication-competent adenovirus type 4 (Ad4) expressing a SARS-COV-2 spike protein and its use as an immunogenic composition for inhibiting SARS-COV-2 infection and transmission.

### BACKGROUND

Coronaviruses are a large family of viruses that typically cause mild to moderate upper respiratory tract disease; however, some members of this family can cause severe disease and death in humans. In the last 20 years, coronaviruses have caused three major outbreaks in humans resulting from severe acute respiratory syndrome coronavirus (SARS-COV), Middle East respiratory syndrome coronavirus (MERS-COV), and SARS-COV-2, the latter of which first emerged in Wuhan, China in December 2019. As of January 2021, SARS-COV-2 had infected more than 84 million people worldwide, leading to nearly 2 million deaths. Although several SARS-COV-2 vaccines have been approved for use in the U.S. and other countries, a need remains for an effective SARS-COV-2 vaccine that induces mucosal immunity and can be rapidly produced in large quantities.

### SUMMARY

Disclosed herein are immunogenic compositions comprised of a replication-competent adenovirus type 4 (Ad4) expressing a SARS-COV-2 spike (S) protein (“Ad4-Spike”), such as a wild-type or modified version of the S protein from the original Wuhan strain or from a SARS-CoV-2 variant, such as the beta (B.1.351) variant, the delta (B.1.617.2) variant, the gamma (P.1) variant, the delta plus variant, or the omicron (B.1.1.529) variant. In the disclosed Ad4 vector, the gene encoding the SARS-COV-2 S protein is cloned into the E3 region of an Ad4 vaccine strain. To accommodate insertion of the S protein, at least a portion of the E3 region is deleted. The disclosed Ad4-Spike vaccines possess several important advantages over other proposed and licensed SARS-COV-2 vaccine platforms. In particular, as a replicating vector, Ad4-Spike is capable of inducing a durable immune response, including mucosal immunity, which is an important factor for inhibiting both infection and transmission of the virus. Furthermore, Ad4-Spike vaccines can be rapidly produced to high titers at a relatively low cost.

Provided herein is a recombinant, replication-competent Ad4 expressing a SARS-COV-2 S protein. The genome of the recombinant Ad4 includes a deletion in the adenovirus E3 region and an insertion of a coding sequence for the SARS-COV-2 S protein. The SARS-COV-2 S protein can be a native S protein or a modified S protein, such as a stabilized or truncated S protein. Additionally, the S protein can be from the Wuhan strain of SARS-COV-2 or a variant thereof, such as a variant of concern (VOC).

Also provided is a recombinant, replication-competent Ad4 vector having a deletion in the adenovirus E3 region and an insertion of a coding sequence for the SARS-COV-2 S protein. The SARS-COV-2 S protein can be a native S protein or a modified S protein, such as a stabilized or truncated S protein, derived from either the Wuhan strain or a SARS-COV-2 variant, such as a VOC.

Further provided are immunogenic compositions that include a recombinant Ad4 or a recombinant Ad4 vector disclosed herein, and a pharmaceutically acceptable carrier.

Also provided are methods of eliciting an immune response against SARS-COV-2 in a subject and methods of immunizing a subject against SARS-COV-2 infection by administering to the subject a therapeutically effective amount of a recombinant Ad4, a recombinant Ad4 vector, or an immunogenic composition disclosed herein. In some embodiments, the recombinant Ad4, recombinant Ad4 vector or immunogenic composition is administered to the upper respiratory tract, such as intranasally.

The foregoing and other objects and features of the disclosure will become more apparent from the following detailed description, which proceeds with reference to the accompanying figures.

### BRIEF DESCRIPTION OF THE DRAWINGS

FIG. 1: SARS-COV-2 spike expression of stabilized and truncated designs in transfected A549 Cells. A549 cells were transfected with a shuttle vector plasmid containing the gene for the SARS-COV-2 spike protein from the Wuhan strain (nCOV). Four spike protein constructs were made: wild-type (WT), stabilized (PP), tail truncated (TT), and endocytosis motif truncated (noEndo). Controls included untransfected (unTF) cells and cells transfected with a plasmid expressing an HIV-1 envelope (Env) protein (FDE3). Expression of spike and Env was measured by flow cytometry using a SARS-COV-2 spike protein-specific antibody and an HIV-1 Env-specific antibody (VRC01), respectively. SARS-COV-2 spike protein expression in transfected A549 cells diminished with stabilizing mutations, truncation of the tail, and truncation of the endocytosis motif, relative to wild-type spike protein.

FIGS. 2A-2B: SARS-COV-2 spike expression of stabilized and truncated designs in infected A549 Cells. Replicating adenovirus carrying a SARS-COV-2 protein gene was used to infect A549 cells. Three spike protein designs based on the Wuhan strain were tested for expression on the surface of A549 cells: wild-type (nCOV-WT), PP-stabilized (nCOV-PP), and tail-truncated (nCOV-TT) spike protein. A replicating adenovirus expressing an HIV-1 Env protein (FDE3) was used as a positive control of infection and uninfected (unIF) cells were used a negative control. Expression of spike protein was measured by flow cytometry using a SARS-COV-2 spike protein-specific antibody. Antibody VRC01 was used to detect expression of HIV Env. Expression of spike by nCOV-WT is shown in FIG. 2A; expression of spike by FDE3, nCOV-PP and nCOV-TT is shown in FIG. 2B. As shown in FIGS. 2A-2B, expression of spike protein was high from both the nCOV-WT and nCOV-PP constructs.

FIG. 3: Immunization with replicating Ad4 containing SARS-COV-2 spike protein gene induces neutralization in rabbits. New Zealand white rabbits were immunized on day 0 and day 28 (indicated by the arrows) with 1.29×10⁹ infectious units (IFU) of purified replicating Ad4 nCoV-WT. Using a luciferase assay, serum neutralization against Wuhan SARS-COV-2 pseudovirus was detected starting at 4 weeks post-immunization (prior to the second dose), and continued to increase up to 12 weeks post-immunization.

FIG. 4: Amino acid alignment of nCOV-PP, nCOV-WT, nCOV-Tail-Truncation, and nCoV-No-Endo spike proteins. Alignment displays locations of three mutations introduced to the SARS-Cov-2 wild-type (Wuhan) spike protein. nCOV-PP contains double proline stabilization substitutions at amino acid position 986 and 987; nCOV-Tail-Truncation includes a deletion of the terminal 24 amino acids at the cytoplasmic tail; and nCOV-No-Endo contains a deletion of the terminal endocytosis signaling motif (terminal five residues). Amino acid numbering is with reference to wild-type spike protein set forth herein as SEQ ID NO: 2.

FIGS. 5A-5B: Serum neutralization against Wuhan pseudovirus in a dose titration of intranasal Ad4-SARS-COV-2_{WuPP} in hamsters. Syrian golden hamsters were intranasally administered 102 to 107 infection forming units (IFU) of Ad4-SARS-COV-2 Wuhan spike with PP stabilization (Ad4-SARS-COV-2_{WuPP}). Serum neutralization against Wuhan pseudovirus was measured at week 4 (FIG. 5A) and week 8 (FIG. 5B). Strong neutralization was observed at both timepoints for the highest doses of Ad4-SARS-COV-2_{WuPP}.

FIGS. 6A-6E: Serum neutralization of intranasal Ad4-SARS-COV-2 expressing the indicated VOC spike in hamsters. Syrian golden hamsters were immunized with intranasal Ad4 expressing stabilized spike proteins from either the Wuhan strain (Ad4-CoV2-Wuhan), the beta variant (Ad4-CoV2-SA), the delta variant (Ad4-CoV2-Indian) or the gamma variant (Ad4-CoV2-Brazil), or a stabilized chimeric spike protein having the beta variant RBD (Ad-CoV2-Wu/RBD-SA). An Ad4 expressing an influenza virus H5 hemagglutinin (Ad4-H5) and sham inoculation were included as negative controls. Serum neutralization against Wuhan pseudovirus (FIG. 6A) or delta pseudovirus (FIG. 6B) was determined 28 days following intranasal administration. In addition, serum neutralization against Wuhan pseudovirus (FIG. 6C), delta pseudovirus (FIG. 6D) and omicron pseudovirus (FIG. 6E) was determined 56 days following intranasal administration.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Jan. 14, 2022, 199 KB, which is incorporated by reference herein. In the accompanying sequence listing:

SEQ ID NO: 1 is the nucleotide sequence
of the Ad4-SARS-CoV-2 spike vector.
TAAATTTAAATGAATTCCGTCAAGGGCGACACAAAAGGTATTCTAAAT
GCATAATAAATACTGATAACATCTTATAGTTTGTATTATATTTTGTAT
TATCGTTGACATGTATAATTTTGATATCAAAAACTGATTTTCCCTTTA
TTATTTTCGAGATTTATTTTCTTAATTCTCTTTAACAAACTAGAAATA
TTGTATATACAAAAAATCATAAATAATAGATGAATAGTTTAATTATAG
GTGTTCATCAATCGAAAAAGCAACGTATCTTATTTAAAGTGCGTTGCT
TTTTTCTCATTTATAAGGTTAAATAATTCTCATATATCAAGCAAAGTG
ACAGGCGCCCTTAAATATTCTGACAAATGCTCTTTCCCTAAACTCCCC
CCATAAAAAAACCCGCCGAAGCGGGTTTTTACGTTATTTGCGGATTAA
CGATTACTCGTTATCAGAACCGCCCAGGATGCCTGGCAGTTCCCTACT
CTCGCCGCTGCGCTCGGTCGTTCGGCTGCGGGACCTCAGCGCTAGCGG
AGTGTATACTGGCTTACTATGTTGGCACTGATGAGGGTGTCAGTGAAG
TGCTTCATGTGGCAGGAGAAAAAAGGCTGCACCGGTGCGTCAGCAGAA
TATGTGATACAGGATATATTCCGCTTCCTCGCTCACTGACTCGCTACG
CTCGGTCGTTCGACTGCGGCGAGCGGAAATGGCTTACGAACGGGGCGG
AGATTTCCTGGAAGATGCCAGGAAGATACTTAACAGGGAAGTGAGAGG
GCCGCGGCAAAGCCGTTTTTCCATAGGCTCCGCCCCCCTGACAAGCAT
CACGAAATCTGACGCTCAAATCAGTGGTGGCGAAACCCGACAGGACTA
TAAAGATACCAGGCGTTTCCCCCTGGCGGCTCCCTCGTGCGCTCTCCT
GTTCCTGCCTTTCGGTTTACCGGTGTCATTCCGCTGTTATGGCCGCGT
TTGTCTCATTCCACGCCTGACACTCAGTTCCGGGTAGGCAGTTCGCTC
CAAGCTGGACTGTATGCACGAACCCCCCGTTCAGTCCGACCGCTGCGC
CTTATCCGGTAACTATCGTCTTGAGTCCAACCCGGAAAGACATGCAAA
AGCACCACTGGCAGCAGCCACTGGTAATTGATTTAGAGGAGTTAGTCT
TGAAGTCATGCGCCGGTTAAGGCTAAACTGAAAGGACAAGTTTTGGTG
ACTGCGCTCCTCCAAGCCAGTTACCTCGGTTCAAAGAGTTGGTAGCTC
AGAGAACCTTCGAAAAACCGCCCTGCAAGGCGGTTTTTTCGTTTTCAG
AGCAAGAGATTACGCGCAGACCAAAACGATCTCAAGAAGATCATCTTA
TTAAGCTTAGAAAAACTCATCGAGCATCAAATGAAATTGCAATTTATT
CATATCAGGATTATCAATACCATATTTTTGAAAAAGCCGTTTCTGTAA
TGAAGGAGAAAACTCACCGAGGCAGTTCCATAGGATGGCAAGATCCTG
GTATCGGTCTGCGATTCCGACTCGTCCAACATCAATACAACCTATTAA
TTTCCCCTCGTCAAAAATAAGGTTATCAAGTGAGAAATCACCATGAGT
GACGACTGAATCCGGTGAGAATGGCAAAAGTTTATGCATTTCTTTCCA
GACTTGTTCAACAGGCCAGCCATTACGCTCGTCATCAAAATCACTCGC
ATCAACCAAACCGTTATTCATTCGTGATTGCGCCTGAGCGAGGCGAAA
TACGCGATCGCTGTTAAAAGGACAATTACAAACAGGAATCGAGTGCAA
CCGGCGCAGGAACACTGCCAGCGCATCAACAATATTTTCACCTGAATC
AGGATATTCTTCTAATACCTGGAACGCTGTTTTTCCGGGGATCGCAGT
GGTGAGTAACCATGCATCATCAGGAGTACGGATAAAATGCTTGATGGT
CGGAAGTGGCATAAATTCCGTCAGCCAGTTTAGTCTGACCATCTCATC
TGTAACATCATTGGCAACGCTACCTTTGCCATGTTTCAGAAACAACTC
TGGCGCATCGGGCTTCCCATACAAGCGATAGATTGTCGCACCTGATTG
CCCGACATTATCGCGAGCCCATTTATACCCATATAAATCAGCATCCAT
GTTGGAATTTAATCGCGGCCTCGACGTTTCCCGTTGAATATGGCTCAT
ATTCTTCCTTTTTCAATATTATTGAAGCATTTATCAGGGTTATTGTCT
CATGAGCGGATACATATTTGAATGTATTTAGAAAAATAAACAAATAGG
GGTCAGTGTTACAACCAATTAACCAATTCTGAACATTATCGCGAGCCC
ATTTATACCTGAATATGGCTCATAACACCCCTTGTTTGCCTGGCGGCA
GTAGCGCGGTGGTCCCACCTGACCCCATGCCGAACTCAGAAGTGAAAC
GCCGTAGCGCCGATGGTAGTGTGGGGACTCCCCATGCGAGAGTAGGGA
ACTGCCAGGCATCAAATAAAACGAAAGGCTCAGTCGAAAGACTGGGCC
TTTCGCCCGGGCTAATTAGGGGGTGTCGCCCTTATCGCTGAGGATCCA
TTTAAATTTAATTAACATCATCAATAATATACCTTATTTTTTTTGTGT
GAGTTAATATGCAAATAAGGCGTGAAAATTTGGGGATGGGGCGCGCTG
ATTGGCTGTGACAGCGGCGTTCGTTAGGGGCGGGGCAGGTGACGTTTT
GATGACGCGACTATGAGGAGGAGTTAGTTTGCAAGTTCTGGTGGGGAA
AAGTGACGTCAAACGAGGTGTGGTTTAAACACGGAAATACTCAATTTT
CCCACGCTGTCTAACAGGAAATGAGGTGTTTTTGGGCGGATGCAAGTG
AAAACGGACCATTTTCGCGCGAAAACTGAATGAGGAAGTGAAATCTGA
GTAATTTAGTGTTTATGACAGGGAGGAGTATTTGCCGAGGGCCGAGTA
GACTTTGACCGTTTACGTGGGGGTTTCGATTACCGTGTTTTTCACCTA
AAGTTCCGCGTACGGTGTCAAAGTCCGGTGTTTTTACGTAGGTGTCAG
CTGATCGTCAGGGTATTTAAACCTGCGCTCTGCAGTCAAGAGGCCACT
CTTGAGTGCCAGCGAGAAGAGTTTTCTCCTCCGCGCCGCGAGTCAGAT
CTACACTTTGAAATATGAGGCACCTAAGAGACCTGCCCGATGAGGAAA
TTATCATCGCTTCCGGGAGCGAGATTCTGGAACTGGTGGTAAATGCTA
TGATGGGCGACGACCATCCGGAACCCCCCACCCCATTTGAGACACCTT
CGCTGCACGATTTGTATGATCTGGAGGTGGATGTGCCCGAGGACGACC
CCAACGAGAAGGCGGTAAATGATTTATTTAGCGATGCCGCGCTGCTAG
CTGCCGAGGAGGCTTCAAGCCCTAGCTCAGACAGCGACTCTTCACTGC
ATACCCCTAGACACGACAGAGGTGAGAAAGAGATCCCCGGGCTTAAAT
GGGAAAAGATGGACTTGCGTTGCTATGAGGAATGCCTGCCCCCAAGCG
ATGATGAGGACGAGCAGGCGATTCAGAACGCAGCGAGCCATGGAGTGC
AAGCCGTCAGCGAGAGCTTTGCACTGGACTGCCCGCCTTTGCCCGGAC
ACGGCTGTAAGTCTTGTGAATTTCATCGCATCAATACTGGAGATAAAG
CTGTGTTATGTGCACTTTGCTATATGAGAGCGTACAACCATTGTGTTT
ACAGTAAGTGTGATTAAGTGAACTTTAAAGGGAGGCAAAGAGTAGGGT
GACTGGGTGATGACTGGTTTATTTATGTATATCTGTTTTTTATATAGG
TCCCGTTTCTGACGCAGATGATGAGACCCCCACTACAGAGTCCACTTT
GTCACCCCCTGAAATTGGCACGTCTCCATCTGACAATATTGTTAGACC
AGTTCCTGTAAGAGCCACTGGGAGGAGAGCAGCTGTAGAATGTTTGGA
TGATTTGCTTCAGGGTGGAGATGAACCTTTGGACTTGTGTACCCGGAA
ACGCCCCAGGCATTAAGTGCCACACATGTGTGTTTACTTGAGGTGATG
TCAGTATTTATAGGGTGTGGAGTGCAATAAAATATGTGTTGACTTTAA
GTGCGTGGTTTATGACTCAGGGGAGGGGACTTTGGGTATATAAGCAGG
TGCAGACCTGTGTGGTTAGCTCAGAGCGGTATGGAGATTTGGACGGTT
TTGGAAGACTTTCACAAGACTAGGCAGCTGCTAGAGAACGCCTCGAAC
GGAGTCTCTTACCTGTGGAGATTCTGCTTCGGCGGTGACCTAGCTAAG
CTAGTCTATAGGGCCAAACAGGATTATAGGGAACAATTTGAGGATATT
TTGAGAGAGTGTCCTGGTCTTTTTGACGCTCTTAACTTGGGCCATCAG
TCTCACTTTAACCAGAGAATTTCAAGAGCCCTTGACTTTACTACTCCT
GGCAGAACCACTGCAGCAGTAGCCTTTTTTGCTTTTATTTTTGACAAA
TGGAGTCAAGAAACCCATTTCAGCAGGGATTACCAGCTGGATTTCTTA
GCAGTAGCTTTGTGGAGAACATGGAAGTGCCAGCGCCTGAATGCAATC
TCCGGCTACTTGCCGGTACAGCCGCTAGACACTCTGAGGATCCTGAGT
CTCCAGCAGCAGGAGGATCAAGAAGAGAATCCGAGAGCCGGCCTGGAC
CCTCCGGCGGAGGAGTAGCTGACCTGTTTCCTGAACTGCACCGGGTGC
TGACTAGGTCTTCGAGTGGTCGGGAGAGGGGTATTAAGCGGGAGAGGC
ATGATGAGACTAATCACAGAATTGAACTGACTGTGGGTCTGATGAGCC
GCAAGCGTCCAGAAACAGTGTGGTGGTATGAGGTGCAGTCAACTGGCA
CAGATGAGGTGTCAGTCATGCATGAGAGATTTTCCCTAGAACAAGTCA
AGACTTGTTGGTTGGAGCCTGAGGATGATTGGGAGGTAGCCATCAGGA
ATTATGCCAAGCTGGCTCTGAGGCCAGATAGAAAGTACAAGATTACTA
AGCTGATAAATATCAGAAATGCCTGCTACATCTCAGGGAATGGGGCTG
AAGTGGAGATCTGTCTCCAGGATAGAGTGGCTTTCAGATGCTGCATGA
TGAATATGTACCCGGGAGTGGTGGACATGGATGGGGTCACCTTTATGA
ACATGAGGTTCAGGGGAGATGGGTATAATGGGACGGTCTTTATGGCCA
ATACCAAGCTGACAGTGCATGGATGCTCCTTCTTTGGGTTTAATAACA
CCTGCATCGAGGCTTGGGGTCAGGTCGGTGTTAAGGGGTGCAGTTTTT
CAGCCAACTGGATGGGGGTAGTGGGCAGGACCAAGAGTATGCTGTCTG
TGAAGAAATGCTTGTTTGAGAGGTGCCACCTGGGGGTGATGAGCGAGG
GCGAAGCCAGAATCCGCCACTGTGCCTCTACCGAGACGGGCTGTTTTG
TGCTGTGCAAGGGCAATGCCAAGATCAAGCATAATATGATCTGTGGAG
CCTCGGACGAGCGCGGCTACCAGATGCTGACCTGCGCCGGTGGGAACA
GTCATATGCTGGCCGCCGTGCATGTGGCTTCCCATTCCCGCAAGCCCT
GGCCTGAGTTCGAGCACAATGTCATGACCAGGTGCAATATGCATCTGG
GGGCTCGCCGAGGCATGTTTATGCCCTACCAGTGCAACCTGAATTATG
TAAAGGTGCTCCTGGAGCCCGATGTCATGTCCAGAGTGAGCCTGACGG
GGGTGTTTGACATGAATGTGGAAGTGTGGAAGATTCTAAGATATGATG
AATACAAGACCAGGTGTCGAGCCTGCGAGTGCGGAGGGAAGCATGCCA
GGTTCCAGCCCGTGTGTGTGGATGTGACGGAGGACCTGCGACCCGATC
ATTTGGTGTTGTCCTGCACCGGGACGGAGTTCGGCTCCAGTGGGGAAG
AATCTGACTAGAGTGAGTAGTGTTTTGGGGAGGGAGAGGACCTGCATA
AGGGGCAGAATGATTAAAATCTGTGCTTTTCTGTGTGTTGCAGCAGCA
TGAGCGGAAACGGCTCCTTTGAGGGAGGGGTATTCAGCCCTTATCTGA
CGGGGCGTCTCCCCTCCTGGGCGGGAGTGCGTCAAAATGTGATGGGAT
CCACGGTGGACGGCCGGCCCGTACAGCCCGCGAACTCTTCAACCCTGA
CCTATGCAACCCTGAGCTCCTCGTCGGTGGACGCAGCTGCCGCCGCAG
CTGCTGCTTCTGCCGCCAGCGCCGTGCGCGGAATGGCCATGGGCGCCG
GCTATTACGGCACTCTGGTGGCCAACTCGAGTTCCACTAATAATCCCG
CCAGCCTGAACGAGGAGAAGCTGCTGCTGTTGATGGCCCAGCTCGAGG
CCTTGACCCAGCGCCTGGGCGAGCTGACCCAGCAGGTGGCTCAGCTGC
AGGAGCAGACGCGGGCCGCGGTTGCCACGGTGAAATCCAAATAAAAAA
TGAATCAATAAATAAACGGAGACGGTTGTTGATTTTAAAAATCAGAGT
CTGAATCTTTATTTGATTTTTCGCGCACGGTAGGCCCTGGACCACCGG
CCTCGATCATTGAGCACCCGGTGGATCTTTTCCAAGACCCGGTAGAGG
TGGGATTGGATATTGAGGTACATGGGCATGAGCCCGTCCCGGGGGTGA
AGGTAGCTCCATTGCAGGGCCTCGTGCTCGGGGGTGGTGTTGTAAATC
ACCCAGTCATAGCAGGGACGCAGGGCGTGGTGTTGCACAATATCTTTG
AGGAGGAGACTGATGGCCACGGGCAGCCCTTTGGTGTAGGTGTTTACA
AACCTGTTGAGCTGGGAGGGATGCATGCGGGGGGAGATGAGGTGCATC
TTAGCCTGGATCTTCAGATTGGCGATGTTACCGCCCAGATCCCGCCTG
GGATTCATGTTGTGCAGGACCACCAGCACGGTGTATCCGGTGCACTTG
GGGAATTTGTCATGCAACTTGGAAGGGAAGGCATGAAAGAATTTGGAG
ACGCCCTTGTGGCCGCCCAGGTTTTCCATGCACTCATCCATGATAATG
GCTATGGGCCCGTGGGCGGCGGCTTGGGCAAAGACGTTTCGGGGGTCG
GACACATCGTAGTTGTGGTCCTGGGTGAGATCTTCATAGGCCATTTTA
ATGAATTTGGGGCGGAGGGTGCCCGATTGGGGGACGAAGGTACCCTCA
ATCCCGGGGGCGTAGTTTCCCTCACAGATCTGCATCTCCCAGGCCTTA
AGCTCCGAGGGGGGGATCATGTCCACCTGCGGGGCGATAAAGAAAACG
GTTTCCGGGGCGGGGGAGATGAGCTGGGCGGAAAGCAGGTTGCGGAGT
AGCTGGGACTTACCGCAGCCGGTGGGGCCGTAGATAACCCCAATGACC
GGCTGCAGGTGGTAGTTGAGGGAGACACAGCTGCCGTCCTCCCTAAGA
AGGGGGGCCACCTCGTTCATCATTTGGCGCACGTGCATGTTCTCGCGC
ACCAGTTCCGCCAGGAGTCGCTCTCCGCCCAGCGAGAGGAGCTCCTGG
AGCGAGGCGAAGTTTTTCAGCGGCTTGAGCCCGTCGGCCATGGGCATT
TTGGAAAGGGTCTGTTGCAGGAGTTCCAAGCGGTCCCAGAGCTCGGTG
ATGTGCTCTACGGCATCTCGATCCAGCAGACCTCCTCGTTTCGCGGGT
TGGGGCGACTGCGGGAGTAGGGCGCCAGACGATGGGCGTCCAGCGCGG
CCAGGGTCCGGTCCTTCCAGGGTCGCAGCGTCCGCGTCAGGGTGGTCT
CCGTCACGGTAAAGGGGTGCGCGCCGGGCTGGGCGCTTGCGAGGGTGC
GCTTCAGGCTCATCCGGCTGGTCGAGAACCGCTCCCGATCGGCGCCCT
GTGCGTCGGCCAGGTAGCAATTGACCATGAGTTCGTAGTTGAGCGCCT
CGGCCGCGTGGCCTTTGGCGCGGAGCTTACCTTTGGAAGTCTGCCCAC
AGGCGGGACAGAGGAGGGACTTGAGGGCGTAGAGCTTGGGGGCGAGGA
AGACGGACTCGGGGGCGTAGGCGTCCGCGCCGCAGTGGGCGCAGACGG
TCTCGCACTCCACGAGCCAGGTGAGGTCGGGCTGATTGGGATCAAAAA
CCAGTTTTCCGCCGTTCTTTTTGATGCGTTTCTTACCTCTGGTCTCCA
TGAGCTCGTGTCCCCGCTGGGTGACAAAGAGGCTGTCCGTGTCCCCGT
AAACCGACTTTATGGGTCGGTCCTCGAGTGGGACGCCGCGGTCCTCGT
CGTAGAGGAAACCCGACCACTCTGAGACGAAGGCCCGGGTCCAAGCCA
GCACGAAGGAGGCCACGTGGGAGGGATAGCGGTCGTTATCCACCAGCG
GGTCCACCTTCTCCAGTGTATGCAAACACATGTCCCCCTCGTCCACAT
CCAGGAAGGTGATTGGCTTGTAAGTGTAGGCCACGTGACCGGGGGTCC
CGGCCGGGGGGGTATAAAAGGGGGCGGGCCGCTGCTCGTCTTCACTGT
CTTCCGGATCGCTGTCCAGGAGCGCCAGCTGTTGGGGTAGGTATTCCC
TCTCAAAGGCGGGCATGACCTCCGCACTCAGGTTGTCAGTTTCTAGAA
ACGAGGAGGATTTGATATTGACGGTGCCGGCGGAGATGCCTTTCAAGA
GCCCCTCGTCCATCTGGTCAGAAAAGACAATCTTTTTGTTGTCGAGTT
TGGTGGCGAAGGAGCCGTAGAGGGCGTTGGAGAGGAGCTTGGCGATGG
AGCGCATGGTCTGGTTCTTTTCCTTGTCGGCGCGCTCCTTGGCGGCGA
TGTTGAGCTGCACGTACTCGCGCGCCACGCACTTCCATTCGGGGAAGA
CGGTGGTTAGCTCGTCTGGCACGATTCTGACCTGCCAGCCCCGGTTAT
GCAGGGTGATGAGGTCAACGCTGGTGGCCACCTCGCCGCGCAGGGGCT
CGTTGGTCCAGCAGAGGCGGCCGCCCTTGCGCGAGCAGAAGGGGGGCA
GGGGGTCCAGCATAAGCTCGTCGGGGGGGTCAGCATCGATGGTGAAGA
TGCCTGGCAGGAGGTCGGGGTCGAAGTAGCTTATGCAGGTGCCCAGAT
CGTCCAGAGAAGCTTGCCATTCGCGCACGGCCAGCGCGCGCTCGTAGG
GACTAAGGGGCGTGCCCCAGGGCATGGGGTGGGTGAGCGCGGAGGCGT
ACATGCCGCAGATGTCGTAGACGTAGAGGGGCTCATCAAGGATGCCAA
TGTAGGTGGGGTAGCAGCGGCCCCCGCGGATGCTGGCGCGCACGTAGT
CATACAACTCGTGCGAGGGGGCGAGGAGCCCGGCTCCGAGATTGGCGC
GGCTGGGTTTTTCGGCGCGGTAGACGATCTGACGGAAGATGGCGTGGG
AGTTGGAGGAGATGGTGGGTCTTTGGAAGATGTTGAAGTGGGCGTGGG
GCAGGCCGACCGAGTCGCGGATGAAGTGGGCGTAGGAGTCTTGCAGCT
TGGCGACAAGCTCGGCGGTGACGAGGACGTCCAGGGCGCAGTAGTCAA
GGGTCTCTTGGATGATGTCATACTTGAGCTGGCCCTTTTGTTTCCACA
GCTCGCGGTTGAGAAGGAACTCTTCGCGGTCCTTCCAGTACTCTTCAA
GGGGGAACCCGTCCTGGTCGGCACGGTAAGAGCCTAGCATGTAGAACT
GGTTAACGGCCTTGTAGGCGCAGCAGCCCTTCTCCACGGGGAGGGCAT
AGGCCTGGGCGGCCTTGCGCAGGGAGGTGTGCGTGAGGGCGAAGGTGT
CCCTGACCATGACCTTTAGGAACTGGTGCTTGAAGTCGATATCGTCGC
AGCCCCCCTGCTCCCAGAGCTGGAAGTCCGTGCGCTTCTTGTAGGCGG
GGTTGGGCAAAGCGAAAGTAACATCGTTGAAGAGGATCTTGCCCGCGC
GGGGCATAAAGTTGCGAGTGATGCGGAAAGGCTGGGGCACCTCGGCCC
GGTTGTTGATGACCTGGGCGGCGAGCACGATCTCGTCGAAGCCGTTAA
TGTTGTGGCCCACAATGTATAGTTCCACGAACCGCGGGCGGCCCTTGA
CGTGGGGCAGTTTCTTGAGCTCCTCGTAGGTGAGCTCGTCGGGGTCGC
TGAGCCCGTGCTGCTCGAGGGCCCAGTCGGCGAGATGGGGGTTGGCGC
GGAGGAAGGAAGTCCAGAGATCCACGGCCAGGGCGGTTTGCAGACGAT
CCCGGTACTGGCGGAACTGCTGACCCACGGCCATTTTTTCGGGGGTGA
CGCAGTAGAAGGTGCGGGGGTCGCCGTGCCAACGGTCCCATTTTAGCT
GGAGGGCGAGATCAAGGGCGAGCTCAACGAGCCGGTCGTCCCCGGAGA
GTTTCATGACCAGCATGAAGGGGACGAGCTGCTTGCCGAAGGACCCCA
TCCAGGTGTAGGTTTCCACATCGTAGGTGAGGAAGAGCCTTTCGGTGC
GAGGATGCGAGCCGATGGGGAAGAACTGGATCTCCTGCCACCAGTTGG
AGGAATGGCTGTTGATGTGATGGAAGTAGAAATGCCGACGGCGCGCCG
AACATTCGTGCTTGTGTTTATACAAGCGGCCACAGTGCTCGCAACGCT
GCACGGGATGCACGTGCTGCACGAGCTGTACCTGGGTTCCTTTGACGA
GGAATTTCAGTGGGAAGTGGAGTCGTGGCGCCTGCATCTGGTGCTGTA
CTACGTCGTGGTGGTCGGCCTGGCCCTCTTCTGCCTCGATGGTGGTCA
TGCTGACGAGCCCGCGCGGGAGGCAGGTCCAGACCTCGGCGCGAACGG
GTCGGAGAGCGAGGACGAGGGCGCGCAGGCCGGAGCTGTCCAGGGTCC
TGAGACGCTGCGGAGTCAGGTCAGTGGGCAGCGGCGGCGCGCGGTTGA
CTTGCAGGAGTTTTTCAAGGGCGCGCGGGAGGTCCAGATGGTACTTGA
TCTCCACCGCGCCGTTGGTGGCGACGTCGATGGCTTGCAGTGTCCCGT
GCCCCTGGGGAGTGACCACCGTCCCCCGTTTCTTCTTGGCGGGCGGAA
GCGGTTTGGCTTCCATGGTTAAAAGCGGCGGCGAGGACGCGCGCCGGG
CGGTAGGGGCGGCTCGGGACCCGGAGGCAGTGGTGGCAGGGGCACGTC
GGCGCCGCGCGCGGGCAGGTTCTGGTACTGCGCCCGGAGAAGACTGGC
GTGAGCGACGACGCGACGGTTGACGTCCTGGATCTGACGCCTCTGGGT
GAAGGCCACGGGACCCGTGAGTTTGAACCTGAAAGACAGTTCGACAGA
ATCAATCTCGGTATCATTGACGGCGGCCTGCCGCAGAATCTCTTGCAC
GTCGCCCGAGTTGTCCTGGTAGGCAATCTCGGTCATGAACTGCTCGAT
CTCCTCCTCCTGAAGGTCTCCGCGGCCGGCGCGCTCCACGGTGGCCGC
GAGGTCGTTGGAGATGCGGCCCATGAGCTGCGAGAAGGCGTTCATGCC
CGCCTCGTTCCAGACGCGGCTGTAAACCACGGCGCCCTCGGGATCGCG
GGCGCGCATGACCACCTGGGCGAGGTTGAGCTCCACGTGGCGCGCAAA
AACCGCGTAGTTGCAGAGGCGCTGGTAGAGGTAGTTGAGCGTGGTGGC
AATGTGCTCAGTGACAAAGAAGTACATAATCCAGCGGCGGAGCGGCAT
TTCGCTGACGTCGCCCAGGGCTTCCAAGCGCTCCATGGCCTCGTAAAA
GTCCACGGCGAAGTTGAAAAACTGGGAGTTGCGTGCAGATACGGTCAA
GTCCTCCTCCAGAAGACGGATGAGCTCGGCGATGGTGGCGCGCACCTC
GCGCTCGAAGGCTCCCGTGAGTTCCTCCACTTCCTCCTCTTCATCCAC
TAACATCTCTTCTACTTCCTCCTCAGGCGGTGGTGGCGGGGGAGGGGG
CCTGCGTCGCCGGCGGCGCACGGGCAGACGGTCGATGAAACGCTCGAT
GGTCTCGCCGCGCCGGCGTCGCATGGTCTCGGTGACGGCGCGCCCGTC
CTCGCGGGGTCGCAGCGTAAAGACGCCGCCGCGCATCTCCAGGTGGCC
CGGGGGGTCCCCGTTGGGCAGGGAGAGTGCGCTGACGATGCATCTTAT
CAATTGCCCCGTAGGGACTCCGCGCAAGGACCTAAGCGTCTCTAGATC
CACGGGATCTGAAAACCGTTGAACGAAGGCTTCGAGCCAGTCGCAGTC
GCAAGGTAGGCTGAGCACGGTTTCTTCTGGCGGCGGTGGGGTGTGGGC
GGGGGCGATGCTGCTGGTGATGAAGTTGAAATAGGCGGTTCTGAGACG
GCGGATGGTGGCGAGGAGCACCAGGTCTTTGGGCCCGGCTTGCTGGAT
GCGCAGACGGTCGGCCATGCCCCAGGCGTGGTCCTGACACCTGGCCAG
GTCCTTGTAGTAGTCCTGCATGAGCCGCTCCACGGGCACCTCCTCCTC
GCCCGCGCGGCCGTGCATACGCGTGAGCCCAAACCCGCGCTGCGGCTG
GACGAGCGCCAGGTCAGCGACGACGCGCTCGGCGAGGATGGCCTGCTG
GATCTGGGTGAGGGTGGTCTGGAAGTCGTCAAAGTCGACGAAGCGGTG
GTAGGCTCCGGTGTTAATGGTGTAGGAGCAGTTGGCCATGACGGACCA
GTTGACAGTCTGGTGACCGGGCCGCGCGAGCTCGTGGTACTTGAGGCG
CGAGTAGGCGCGCGAGTCGAAGATGTAGTCGTTGCAGGTGCGCACCAG
GTACTGGTAGCCGATGAGGAAGTGCGGCGGCGGCTGGCGGTAGAGCGG
CCATCGCTCGGTGGCGGGGGCGCCGGGCGCTAGGTCCTCGAGCATGGT
GCGGTGGTAGCCGTAGATGTACCTTGACATCCAGGTGATGCCGGCGGC
GGTGGTGGAGGCGCGAGGGAACTCGCGGACGCGGTTCCAGATGTTGCG
CAGCGGCAGGAAGTAGTTCATGGTGGGCACGGTCTGGCCCGTGAGGCG
CGCGCAGTCGTTGATGCTCTAGACATACGGGCAAAAACGAAAGCGGTC
AGCGGCTCGACTCCGTGGCCTGGAGGCTAAGCGAACGGGTTGGGCTGC
GCGTGTACCCCGGTTCGAATCTCGAATCAGGCTGGAGCCGCAGCTAAC
GTGGTACTGGCACTCCCGTCTCGACCCAGGCCTGCACAAAACCTCCAG
GATACGGAGGCGGGTCGTTTTGCAAATTTTTGGCGGTCGAAAAAAGCT
AGTAAGCGCGGAAAGCGGCCGACCGCAATGGCTCACTGCCGTAGATTG
GAGAAGAATCGCCAGGGTTGCGTTGCGGTGTGCCCCGGTTCGAGACCG
CTCGGGTCGGCCGAATTCCGCGGCTAACGAGGGCGTGGCTGCCCCGTC
GTTTCCAAGACCCCATAAGCCAGCCGACTTCTCCAGTTACGGAGCGAG
CCCCTCTTTTGTTTTGTTTTTTGCCAGATGCATCCCGTACTGCGGCAG
ATGCGCCCCCACCCTCCACCGCAACAGCAGCCCCCTCCTACGCAACAG
CCGGCGCTTCTGCCTCCGCCCCAGCAGCAGCAACTTCCAGCCACTACC
GCCGCGGCCGCCGTGAGCGGGGCCGGGCAGAGTCAGTATGACCTGGCT
TTGGAAGAGGGCGAGGGGCTGGCGCGCCTGGGGGCGTCGTCGCCGGAG
CGGCACCCGCGCGTGCAGATGAAAAGGGACGCTCGCGAGGCCTACGTG
CCCAAGCAGAACCTGTTCAGAGACAGGAGCGGCGAGGAGCCCGAGGAG
ATGCGCGCAGCCCGTTTCCACGCGGGGGGGGAGCTGCGGCGCGGCCTG
GACAGAAAGAGGGTGCTGAGGGACGAGGATTTCGAGGCGGACGAGCTG
ACGGGGATCAGCCCTGCGCGCGCGCACGTGGCCGCGGCCAACCTGGTC
ACGGCGTACGAGCAGACCGTGAAGGAGGAGAGCAACTTCCAAAAATCC
TTCAACAACCACGTGCGCACCCTGATCGCGCGCGAGGAGGTGACCCTG
GGCCTGATGCACCTGTGGGACCTGCTGGAGGCCATTGTGCAGAACCCC
ACCAGCAAACCGCTGACGGCGCAGCTGTTCCTGGTGGTGCAGCACAGT
CGGGACAACGAGACTTTTAGGGAGGCGCTGCTGAATATCACCGAGCCC
GAGGGCCGCTGGCTTCTGGACCTGGTGAATATTCTGCAGAGCATCGTG
GTGCAGGAGCGCGGGCTGCCGCTGTCCGAGAAGCTGGCGGCCATCAAC
TTTTCGGTGCTGAGTTTGGGCAAGTACTACGCTAGGAAGATCTACAAG
ACCCCGTACGTGCCCATAGACAAGGAGGTGAAGATCGACGGGTTTTAC
ATGCGCATGACCCTGAAAGTGCTGACCCTGAGCGACGATCTGGGGGTG
TACCGCAACGACAGGATGCGCCGCGCGGTAAGCGCCAGCAGGCGGCGC
GAGCTGAGCGATCAGGAGCTGATGCACAGCCTGCAGCGGGCCCTGACC
GGGGCCGGGACCGAGGGGGAGAGCTACTTTGACATGGGCGCGGACCTG
CACTGGCAGCCCAGCCGCCGGGTCTTGGAAGCCGCGGCGGTCCCTTAC
GTAGAAGAGGTGGACGATGAGGATGAGGGCGAGTACCTGGAAGACTGA
TGGCGCGACCGTATTTTTGCTAGATGCAGCAACAGCCACCTCCTGATC
CCGCAATGCGGGCGGCGCTGCAGAGCCAGCCGTCCGGCATTAACTCCT
CGGACGATTGGACCCAGGCCATGCAACGCATCATGGCGCTGACGACCC
GCAACCCCGAAGCCTTTAGACAGCAGCCCCAGGCCAACCGGCTCTCGG
CCATCCTGGAGGCCGTGGTGCCCTCGCGCTCCAACCCCACGCACGAGA
AGGTGCTGGCCATCGTGAACGCGCTGGTTGAGAACAAGGCCATTCGCG
GCGACGAGGCCGGGCTGGTGTACAACGCACTGCTGGAGCGCGTGGCCC
GCTACAACAGCACCAACGTGCAGACCAACCTGGACCGCATGGTGACCG
ACGTGCGCGAAGCCGTGGCCCAGCGCGAACGGTTCCACCGCGAGTCCA
ACCTGGGATCCATGGTGGCACTGAACGCCTTCCTCAGCACGCAGCCCG
CCAACGTGCCCCGGGGCCAGGAGGACTACACCAACTTCATTAGCGCCC
TGCGGCTAATGGTGACCGAGGTGCCCCAGAGCGAGGTGTACCAGTCGG
GCCCGGACTACTTCTTCCAGACCAGTCGCCAGGGCTTGCAGACCGTGA
ACCTGAGTCAGGCTTTCAAGAACTTGCAGGGACTGTGGGGCGTGCAGG
CTCCGGTCGGGGACCGCGCGACGGTGTCGAGCCTGCTGACGCCGAACT
CGCGCCTGCTGCTGCTGCTGGTGGCGCCCTTCACGGACAGCGGTAGTA
TCAACCGCAACTCGTACCTGGGCTACCTGATTAACCTGTACCGCGAGG
CCATTGGCCAGGCGCACGTGGACGAGCAGACCTACCAGGAGATTACCC
ACGTGAGCCGCGCCCTTGGCCAGGACGACCCGGGCAATCTGGAAGCCA
CCCTGAACTTCTTGCTGACCAACCGGTCGCAGAAGATCCCGCCCCAGT
ACGCGCTGAGCGCCGAGGAGGAGCGTATATTGAGATACGTGCAGCAAA
GTGTGGGACTGTTCCTGATGCAGGAGGGGGCCACCCCCAGCGCCGCGC
TCGACATGACCGCGCGCAACATGGAGCCCAGCATGTACGCCAGTAATC
GCCCGTTTATTAATAAGCTGATGGACTACCTGCATCGGGCGGCCGCCA
TGAACTCTGACTATTTCACCAACGCCATCCTGAACCCCCACTGGCTCC
CGCCGCCGGGGTTCTACACGGGCGAGTACGACATGCCCGACCCCAATG
ACGGGTTTCTGTGGGACGACGTGGACAGCAGCGTGTTCTCCCCCCGAC
CGGGTGCTAACGAGCGCCCCTTGTGGAAGAAAGAGGGCAGCGACCGGC
GCCCGTCCTCGGCGCTGTCCGGCCGCACGGGTGCTGCCGCAGCGGTGC
CCGAGGCCGCCAGTCCCTTTCCGAGCTTGTCACTGAACAGCGTCCGCA
GTAGCGAGCTGGGCAGGATCACGCGCCCGCGCTTGCTGGGCGAGGAGG
AGTACTTAAATAACTCGCTGTTGAGGCCCGAGCGGGAGAAGAACTTCC
CCAATAACGGGATAGAGAGTCTGGTGGATAAGATGAGCCGCTGGAAGA
CGTACGCGCATGAGCACAGGGACGATCCCCGGGCAACGCAGGGGGCCA
CCAGCCGGGGCAGTGCCGCCCGTAAACGCCGCTGGCACGACAGGCAGC
GGGGACTGATGTGGGACGATGAGGATTCCGCCGACGACAGCAGCGTGT
TGGACTTGGGCGGGAGTGGTGGTGGTAACCCGTTCGCTCACCTGCGCC
CCCGCGTCGGGCGCCTGATGTAAAAAGAAACCAAAAATAAATGGTACT
CACCAAGGCCATGGCGACCAGCGTGCGTTCGTTTCTTCTCTGTTGTAT
CTAGTATGATGAGGCGTGCGTACCCGGAGGGTCCTCCTCCCTCGTACG
AGAGCGTGATGCAGCAGGCAATGGCGGCGGCGGCGGCGATGCAGCCCC
CGCTGGAGGCTCCTTACGTGCCACCGCGGTACCTGGCGCCTACGGAGG
GGCGAAACAGCATTCGTTACTCGGAGCTGGCACCCTTGTACGATACCA
CCCGGTTGTACCTGGTGGACAACAAGTCGGCGGACATCGCCTCGCTGA
ACTACCAGAACGACCACAGCAACTTTCTGACCACCGTGGTGCAGAACA
ACGATTTCACCCCCACGGAGGCCAGCACCCAGACCATCAACTTTGACG
AGCGCTCGCGGTGGGGCGGTCAGCTGAAAACCATCATGCATACCAACA
TGCCCAACGTGAACGAGTTCATGTACAGCAACAAGTTCAAGGCGCGGG
TCATGGTCTCCCGCAAGACCCCCAACGGGGTGACAGTAGGGGATGATT
ATGATGGTAGTCAGGATGAGCTGAAATACGAGTGGGTGGAGTTTGAGC
TGCCCGAAGGCAACTTCTCGGTGACCATGACCATTGACCTGATGAACA
ACGCCATCATCGACAATTACTTGGCAGTGGGGCGGCAGAACGGGGTGC
TGGAGAGCGACATCGGCGTGAAGTTCGACACCCGGAACTTCAGGCTGG
GTTGGGACCCCGTGACCGAGCTGGTCATGCCCGGGGTGTACACCAACG
AGGCCTTCCACCCCGACATCGTGCTGTTGCCCGGCTGCGGGGTGGACT
TTACCGAGAGCCGCCTCAGTAATATGCTGGGCATCCGCAAGAGGCAGC
CCTTCCAGGAGGGTTTCCAGATCATGTACGAGGACCTGGATGGAGGTA
ACATCCCCGCGCTCTTGGATGTCGAGGCCTATGAGAAAAGCAAGGAGG
AGAGCGTCGCCGCGTCAACCGCAGCCGTAGCCACCGCCTCTACCGAGG
TCCGGGGCGATAATTTTGCTAGCGCCGCAGCAGTGGCGGCGGCCAAGG
CTGATGAAACCGAAAGTAAGATAGTTATTCAGCCGGTGGAGAAGGATA
GCAAGGATAGGAGCTACAACGTGCTCTCGGACAAGAAAAACACCGCCT
ACCGCAGCTGGTACCTGGCCTACAACTATGGCGACCACGAGAAGGGCG
TGCGCTCCTGGACGCTGCTCACCACCTCGGACGTCACCTGCGGCGTGG
AGCAAGTCTACTGGTCGCTGCCCGACATGATGCAAGACCCGGTCACCT
TCCGCTCCACGCGTCAAGTTAGCAACTACCCGGTGGTGGGCGCCGAGC
TCATGCCCGTCTACTCCAAGAGCTTCTTCAACGAGCAGGCCGTCTACT
CGCAGCAGCTGCGCGCCTTCACCTCGCTCACGCACGTCTTCAACCGCT
TCCCTGAGAACCAGATCCTCGTCCGCCCGCCCGCGCCCACCATTACCA
CCGTCAGTGAAAACGTTCCTGCTCTCACAGATCACGGGACCCTGCCGC
TGCGCAGCAGTATCCGGGGAGTCCAGCGCGTGACCGTTACTGACGCCA
GACGCCGCACCTGCCCCTACGTCTACAAGGCCCTGGGCATAGTCGCGC
CGCGCGTCCTCTCGAGCCGCACCTTCTAAAAAATGTCCATTCTCATCT
CGCCCAGTAATAACACCGGTTGGGGTCTGCGCGCGCCCAGCAAGATGT
ACGGAGGCGCTCGCCAACGCTCCACGCAACACCCCGTGCGCGTGCGCG
GGCACTTCCGCGCTCCCTGGGGCGCCCTCAAGGGCCGCGTGCGGTCGC
GCACCACCGTCGACGACGTGATCGACCAGGTGGTGGCCGACGCTCGCA
ACTACACCCCCGCCGCCGCGCCCGTCTCCACCGTGGACGCCGTCATTG
ACAGCGTGGTGTCCGACGCGCGCCGGTACGCCCGCGCCAAGAGCCGGC
GGCGGCGCATCGCCCGGCGGCACCGTAGCACCACCGCCATGCGTGCGG
CGCGAGCCTTGCTGCGCAGGGCCAGGCGCACGGGACGCAGGGCCATGC
TCAGGGCGGCCAGACGCGCGGCTTCAGGCGCCAGCGCCGGCAGGACTC
GGAGACGCGCGGCCACGGCGGCGGCAGCGGCCATAGCCAGCATGTCCC
GCCCGCGGCGAGGGAACGTGTACTGGGTGCGCGACGCCGCCACCGGTG
TGCGCGTGCCCGTGCGCACCCGCCCCCCTCGCACTTGAAGATGTTCAC
TTCGCGATGTTGATGTGTCCCAGCGGCGAGGAGAAGGATGTCCAAGCG
CAAATTCAAGGAAGAGATGCTCCAGGTCATCGCGCCTGAGATCTACGG
CCCCGCGGCGGCGGTGAAGGATGAAAGAAATCCCCGCAAAATCAAGCG
GGTCAAAAAGGACAAAAAGGAAGAAGATGATGTGGACGATATGGTAGA
GTTTGTGCGCGAGTTTGCCCCCCGGAGGCGCGTGCAGTGGCGCGGGCG
GAAAGTGCGTCCGGTGCTGAGACCCGGCACCACGGTGGTTTTCGCGCC
TGGCGAGCGGTCCGGCACGACATCCAAGCGCTCCTACGATGAGGTGTA
CGGGGACGAGGATATTCTCGAGCAGGCGGCCGAGCGCCTGGGCGAGTT
TGCTTACGGCAAGCGCAACCGCCTTGCGCCCCTGAAGGAAGAGGTGGT
GTCCATCCCGCTGGACCACGGCAACCCCACGCCGAGTCTTAAGCCCGT
GACCCTGCAGCAGGTGCTGCCGAGCGCGGCGCCGCGTCGGGGCTTGAA
GCGCGAGGGCGAGGATGTGTACCCCACCATGCAGCTGATGGTGCCCAA
GCGCCAGAAGCTGGAAGACGTGCTGGAGACCATGAAGGTGGACCCGGA
CGTGCAGCCCGAGGTCAAGGTGAGGCCCATCAAGCAGGTGGCCCCGGG
CCTTGGCGTGCAGACCGTGGACATCAAGATCCCCACGGAGCCCATGGA
AACGCAGACCGAGGTCGTGAAGCCCATCACCAGCACCATGGAGGTGCA
GACGGATCCTTGGATGCCGGCGGCGCCCCGAAAACCCCGGCGCAAGTA
CGGCGCGGCCAGCCTGCTGATGCCCAACTACGCGCTGCATCCTTCCAT
CATCCCCACGCCGGGCTACCGCGGCACGCGCTTCTACCACGGCTATAC
CGGCTCCCGCCGCCGCAAGACCACCACCCGCCGCCGTCGTCGCCGCAC
AGCTGCAACTCCCGCTGCCGCCCTGGTGCGGAGAGTGTACCGCCGCGG
CCGCGCGCCTCTGACCCTGCCGCGGGCGCGCTACCACCCGAGCATTAC
CATTTAACTTTGCCGTCGCCTTTGCAGATATGGCTCTCACATGCCGCA
TTCGCGTCCCCATTACGGGCTACCGAGGAAGAAAACCGCGCCGTAGAA
GGCTGGCGGGAAGCGGGATGCGCCGCCACCCCCACCGGCGGCGGCGCG
CCATCAGCAAGCGGTTGGGGGGAGGCTTCCTGCCCGCGCTGATCCCCA
TCATCGCCGCGGCGATCGGGGCGATCCCCGGCATTGCTTCCGTGGCGG
TGCAGGCCTCTCAGCGCCACTGAGACACACACTTGGAAATTGTAATAA
ACCCGAATGGACTCTGACGCTCCTGGTCCTGTGATGTGTTTTTGTAGA
CAGATGGAAGACATCAATTTTTCGTCCCTGGCTCCGCGACACGGCACG
CGGCCGTTTATGGGCACCTGGAGCGACATCGGCACCAGCCAACTGAAC
GGGGGCGCCTTCAATTGGAGCAGTCTCTGGAGCGGGCTTAAGAATTTT
GGGTCCACGCTTAAAACCTATGGCAGCAAGGCGTGGAACAGCACCACA
GGGCAGGCGCTGAGAGATAAGCTGAAAGAGCAGAACTTCCAGCAGAAG
GTAGTCGATGGCCTCGCCTCAGGCATCAACGGGGTGGTGGACCTGGCC
AATCAGGCCGTGCAGCGGCAGATCAACAGCCGCCTGGACCCGGTTCCC
CCCGCCGGCTCCGTGGAGATGCCGCAGGTGGAGGAGGAGCTGCCTCCC
CTGGACAAGCGGGGCGACAAGCGTCCCCGTCCCGACGCGGAGGAGACG
CTGCTGACGCACACGGACGAACCGCCCCCGTACGAGGAGGCGGTGAAA
CTGGGCCTGCCCACCACGCGTCCCATTGCGCCTCTAGCTACCGGGGTG
CTGAAACCCGAGAGTAGTAAGCCCGCGACCTTGGACTTGCCTCCTCCG
CCCACTCCCCGCCCCTCCACAGTGGCTAAGCCCCTGCCGCCGGTGGCC
GTGGCCCGCGCGCGACCGGGGGCTCGCCCTCAGGCGAACTGGCAGAGC
ACTCTGAACAGCATCGTGGGTCTGGGAGTGCAGAGTGTGAAGCGCCGC
CGCTGTTATTAAAAAACACTGTAGCGCTTAACTTGCTTGTCTGTGTAT
ATGTGTATGTCCGCCGCCGCTGCTGTCCAGAAGGAGGAGTGAAGAGAA
AGGCGCGTCGTCGAGTTGCAAGATGGCCACCCCATCGATGCTGCCCCA
GTGGGCGTACATGCACATCGCCGGACAGGACGCTTCGGAGTACCTGAG
TCCGGGTCTGGTGCAGTTCGCCCGCGCCACAGACACCTACTTCAGTCT
GGGGAACAAGTTTAGGAACCCCACGGTGGCGCCTACCCACGATGTGAC
CACCGACCGCAGCCAGCGGCTGACGCTGCGCTTTGTGCCCGTGGACCG
GGAGGACAACACCTACTCGTACAAAGTGCGCTACACGCTGGCCGTGGG
CGACAACCGCGTGCTGGACATGGCCAGCACCTACTTTGACATCCGCGG
CGTGCTGGATCGGGGCCCTAGCTTCAAACCCTACTCCGGCACTGCCTA
CAACAGCCTGGCTCCCAAGGGAGCGCCCAACACCTGCCAGTGGAAGGA
TTCTGACAGCAAAATGCATACCTTTGGGGCAGCTGCCATGCCCGGTGT
TACTGGGAAAAAGATAGAAGCTGATGGGCTGCCTATTAGAATAGATTC
AACTTCTGGAACTGACACAGTAATTTATGCTGATAAAACTTTCCAACC
AGAACCACAAGTTGGAAATGACAGTTGGGTTGACACCAATGGTGCAGA
GGAAAAATATGGAGGCAGAGCTCTAAAGGACACTACAAAAATGAAACC
CTGTTATGGTTCATTCGCCAAGCCTACCAACAAAGAAGGTGGTCAGGC
TAACTTAAAAGATTCAGAACCCGCCGCCACCACTCCTAACTATGATAT
AGACCTGGCTTTCTTTGACAGCAAAACTATTGTTGCTAACTACGATCC
AGATATTGTAATGTACACAGAAAATGTTGACTTGCAGACTCCAGATAC
TCATATTGTATACAAACCTGGAACAGAGGACACCAGCTCTGAATCCAA
TTTGGGTCAGCAGGCCATGCCTAACAGACCCAACTACATTGGCTTCAG
AGACAATTTTATCGGGCTCATGTACTACAACAGCACTGGCAATATGGG
GGTGCTGGCCGGTCAGGCCTCTCAGCTGAATGCTGTGGTTGACTTGCA
AGACAGAAACACTGAACTGTCCTACCAGCTCTTGCTTGACTCTCTGGG
TGACAGAACCCGGTATTTCAGTATGTGGAATCAGGCGGTGGACAGCTA
TGATCCTGATGTGCGCATTATTGAAAACCATGGTGTGGAGGATGAATT
GCCAAACTATTGCTTTCCGTTGAATGGTGTGGGATTGACAGACACTTA
CCAGGGTGTTAAAGTTAAAACAGATGCAGGTTCTGAAAAGTGGGACAA
AGATGACACCACAGTTAGTAATGCTAATGAAATCCATGTAGGCAATCC
TTTTGCCATGGAAATCAACATCCAAGCCAACCTGTGGAGGAACTTCCT
CTATGCCAATGTTGCCCTCTATTTGCCTGATAAATACAAATACACACC
GGCCAACATCACCCTGCCCACCAACACCAACACCTACGAGTACATGAA
CGGCCGGGTGGTGGCGCCCTCGCTGGTGGACGCCTACATTAACATTGG
GGCGCGCTGGTCGCTGGACCCCATGGACAACGTAAATCCCTTCAACCA
CCACCGCAATGCGGGCTTGCGCTACCGCTCCATGCTCCTGGGCAACGG
GCGCTACGTGCCATTCCACATCCAGGTGCCCCAGAAATTTTTTGCCAT
TAAGAGCCTCCTGCTCCTGCCCGGGTCCTACACCTACGAGTGGAACTT
CCGCAAGGACGTCAACATGATCCTGCAGAGTTCCCTTGGCAACGACCT
GCGCACAGACGGGGCCTCCATCACCTTCACCAGCATTAACCTCTACGC
CACCTTCTTCCCCATGGCGCACAACACCGCCTCCACGCTTGAGGCCAT
GCTGCGCAACGACACCAATGACCAATCCTTCAACGACTACCTCTCGGC
GGCCAACATGCTCTATCCCATCCCGGCCAACGCCACCAACGTGCCCAT
CTCCATCCCCTCGCGCAACTGGGCCGCCTTTCGCGGCTGGTCCTTCAC
GCGTCTCAAGACCAAAGAGACGCCCTCGCTGGGCTCCGGGTTCGACCC
CTACTTCGTCTACTCGGGCTCCATCCCCTACCTCGACGGCACCTTCTA
CCTCAACCACACCTTCAAGAAGGTCTCCATCACCTTCGACTCTTCCGT
CAGCTGGCCCGGCAACGACCGGCTCCTGACGCCCAACGAGTTCGAAAT
CAAGCGCACCGTCGACGGCGAGGGATACAACGTGGCCCAGTGCAACAT
GACCAAGGACTGGTTCCTGGTCCAGATGCTGGCCCACTACAACATCGG
CTACCAGGGCTTCTACGTGCCCGAGGGCTACAAGGACCGCATGTACTC
CTTCTTCCGCAACTTCCAGCCCATGAGCCGCCAGGTGGTGGACGAGGT
TAACTACAAGGACTACCAGGCCGTCACCCTGGCCTACCAACACAACAA
CTCGGGCTTCGTTGGATACCTCGCGCCCACTATGCGCCAGGGCCAGCC
CTACCCCGCCAACTACCCCTACCCGCTCATCGGCAAGAGCGCCGTTAC
CAGCGTCACCCAGAAAAAGTTCATCTGCGACAGGGTCATGTGGCGCAT
CCCCTTCTCCAGCAACTTCATGTCCATGGGCGCGCTCACCGACCTCGG
CCAGAACATGCTCTATGCTAACTCCGCCCACGCGCTAGACATGAATTT
CGAAGTCGACCCCATGGATGAGTCCACCCTTCTCTATGTTGTCTTCGA
AGTCTTCGACGTCGTCCGAGTGCACCAGCCCCACCGCGGCGTCATTGA
GGCCGTCTACCTGCGCACCCCCTTCTCAGCCGGTAACGCCACCACATA
AATTCTTGCTTCTTGCAAGAAGCCATGGCCGCGGGCTCCGGCGAGCAG
GAGCTCAGGGCCATCATCCGCGACCTGGGGTGCGGGCCCTACTTCCTG
GGCACCTTCGATAAGCGATTCCCGGGATTCATGGCCCCGCACAAGGTG
GCCTGCGCCATCGTCAACACGGCCGGCCGCGAGACCGGGGGCGAGCAT
TGGCTGGCCTTCGCCTGGAACCCGCGCTCGAACACCTGCTACCTCTTC
GACCCCTTCGGGTTCTCGGACCAGCGCCTCAAGCAAATCTACCAGTTC
GAGTACGAGGGACTGCTGCGCCGCAGCGCCCTGGCCACCAAGGACCGC
TGCGTTACCCTGGAAAAGTCCACCCAGACCGTGCAGGGTCCGCGTTCG
GCCGCCTGCGGGCTTTTCTGCTGCATGTTCCTACACGCCTTCGTGCAC
TGGCCCAACCGCCCCATGGACAAAAATCCCACCATGAACTTGCTGACG
GGGGTGCCCAACGGCATGCTCCAGTCGCCCCAGGTGGAACCTACCCTG
CGCCGCAACCAGGAGGCACTCTACCGCTTCCTCAACTCCCACTCTGCA
TACTTTCGCTCTCACCGCGCGCGCATTGAGAAGGCCACCGCCTTCGAC
CGCATGAATCAAGACATGTAACAGTGTGTTTTAAAATATGTTTAATAA
ACAGCACTTTTTATGTGACACATGCATTTGAGATAATTTTATTCTTAA
AAATCGAAGGGGTTCTGCCGGGAGGTTTCGGCATGGCCCGCGGGCAGG
GACACGTTGCGGAACTGGTACTTGGCCAGCCACTTGAACTCGGGGATC
AGCAGTTTCGGCAGCAGGGTGTCGGGGAACGAGTCGGTCCACAGCTTC
CGCGTCAGTTGCAGGGCGCCCAGCAGGTCGGGCGCGGAGATCTTGAAA
TCGCAGTTGGGACCCGCGTTTTGCGCGCGAGAGTTGCGGTACACAGGG
TTGCAGCACTGGAACACCATCAGGGCCGGATGCTTCACGCTCGCCAGC
ACCGTAGCGTCGGTGATCCCGTCCACGTCGAGGTCTTCGGCGTTGGCC
ATCCCGAAGGGGGTCATCTTGCAGGTCTGCCGGCCCATGGTGGGCACG
CAGCCGGGCTTGTGGTTGCAATCGCAGTGCAGGGGGATCAGCATCATC
TGGGCCTGGTCGGCGTTCATCCCCGGGTACATGGCCTTCATGAAAGCC
TCCAGCTGCTTAAACGCCTGCTGGGCCTTGGCTCCCTCGGTGAAGAAG
ACCCCGCAGGACTTGCTAGAAAACTGGTTGGTAGCGCACCCGGCGTCG
TGCACGCAGCAGCGCGCGTCGTTGTTGGCCAGCTGCACCACGCTGCGC
CCCCAGCGGTTCTGGGTAATCTTGGCCCGGTCGGGGTTCTCCTTTAGC
GCGCGTTGCCCGTTCTCGCTTGCCACATCCATCTCGATCATGTGCTCC
TTCTGGATCATGGTGGTCCCGTGCAGGCACCGCAGCTTGCCCTCGACT
TCGGTACAGCCGTGCAGCCACAGCGCGCACCCCGTGCTCTCCCAGTTC
TTGTGGGCGATCTGGGAATGCGCATGCACGAACCCCTGCAGGAAGCGG
CCCATCATGGTCGTCAGGGTCTTGTTACTGGTAAAGGTCAGCGGAATG
CCGCGGTGCTCCTCGTTGATGTACAGGTGGCAGATGCGGCGATACACC
TCGCCCTGCTCGGGCATCAGTTGGAAGTTGGATTTTAGGTCGCTTTCC
ACACGGTAGCGCTCCATCAGCATATTCATGATTTCCATGCCCTTCTCC
CAGGCCGATACAATGGGCAGGCTCAGGGGGTTCGTCACCGCCATCTTA
GCGCTAGCAGCCTTCGTCAGCGGGTCGTTCTCATTGAGAGTCTCAAAG
CTCCGCTTGCCGTCCTTCTCGGTGATCCGCACGGGGGGGTAGCTGAAG
CCCACGGCCGCCAGCTCCTCCTCGGCCTCTCTTTCGTCCTCGCTGTCC
TGGCTGACGTCCTGCAGGGGCACATGCTTCGTTTTGCGGGGTTTCTTT
TTGGGCGGCTGCTGCGGCGGCGGTGGTTGTTCCTGAGGCGAGGGGGAG
CGCGAGTTCTCGCTCACCACTACTATCTCTTCTTCTTGGTCCGAGGCC
ACGCGGCGGTAGGTATGTCTCTTCAGGGGCAGAGGCGGAGGCGACGGG
CTCTCGCGGCCCGGCGGGTGGCTGGCAGAGCCCCTTCCGCGATCGGGG
GTGCGCTCCCGGCGGCGCTCTAACTGACTTCCTCCGCGGCCGGCCATT
GTGTTCTCCTAGGGAACAACAACAAGCATGGAGACTCAGCCATCGTCG
CCAACCTCGCCATCTGCCCCCACCGCCGACAAGAAGCAGCAGCAGAAT
GAGAGCTTAACCGCCCCGCCGCCCAGCCCCGCCACCTTTGTCGCGGCC
CCAGACATGCAAGAGATGGAGGAATCCATTCAGATTGACCTGGGCTAT
GTGACGCCCGCGGAGCACGAGGAGGAGCTTGCAGTGCGCTTTTCAACC
CAGGAAGAGATACACCAAGAACAGCCAGAGCAGGAAGCAAAGAGCGAG
CATGACTACCTCCACCAGAGCGGGGGGGAGGACGCCCTCATCAAGCAT
CTGGCCCGGCAGGCCATCATCGTCAAGGACGCGCTGCTTGACCGCACC
GAGGTGCCCCTCAGCGTGGAGGAGCTCAGCCGCGCCTACGAGCTCAAC
CTCTTCTCGCCGCGCGTGCCCCCCAAGCGCCAGCCCAACGGCACCTGC
GAGCCCAACCCACGCCTCAACTTCTACCCGGTCTTCGCGGTGCCCGAG
GCCCTGGCCACCTACCACATCTTTTTCAAGAACCAAAGGATCCCTGTC
TCCTGTCGCGCCAACCGCACCCGCGCCGACTCCCTTTTCAACCTGGGC
CCCGGTGCCCGCCTACCTGATATCGCCTCCTTGGAAGAGGTTCCCAAG
ATCTTCGAGGGTCTGGGCAGCGACGAGACTCGGGCCGCAAACGCTCTG
CAAGGAGAAGGAGGAGATCATGAGCACCACAGCGCCCTGGTGGAGTTG
GAAGGCGACAACGCGCGTCTGGCGGTGCTCAAGCGCACGATCGAGCTG
ACCCATTTCGCCTACCCGGCGCTTAACCTGCCCCCCAAAGTCATGAGC
ACGGTTATGGATCAGGTGCTCATCAAGCGCGCGTCGCCCATCTCCAAG
GAGATGCAAGACCCCGAGAGCTCCGAGGAGGGCAAGCCCGTGGTCAGC
GACGAGCAGCTGGCGCGGTGGCTGGGACCCCAAGCTAGTCCCCAGAGC
TTGGAAGAGCGGCGCAAGCTCATAATGGCCGTGGTCCTGGTGACCGCG
GAGCTGGAGTGTCTGCGCCGCTTCTTCGCCGACGCAGAAATTCTGCGC
AAGGTCGAGGAGAACCTGCACTACATCTTCAGGCACGGGTTCGTACGC
CAGGCCTGCAAGATCTCCAACGTGGAGCTGACCAACCTGGTCTCCTAC
ATGGGCATCTTGCACGAGAACCGCCTGGGGCAGAACGTGCTGCACACC
ACCCTGCGCGGGGAGGCCCGCCGCGACTACATCCGCGACTGCGTTTAC
CTCTACCTCTGCCACACCTGGCAGACAGCCATGGGCGTGTGGCAGCAG
TGTCTGGAGGAGCAGAACCTAAAAGAGCTCTGCAAGCTCCTGCAGAAG
AACCTCAAGGCCCTGTGGACCGGGTTCGACGAGCGCACCACCGCCTCG
GACCTGGCAGACCTCATTTTCCCCGAGCGTCTCAGGCTGACGCTGCGC
AACGGTTTGCCCGACTTTATGAGTCAAAGCATGTTGCAAAACTTTCGC
TCTTTCATCCTCGAACGCTCCGGGATCCTGCCGGCCACCTGCTCCGCG
CTGCCCTCGGACTTCGTGCCGCTGACCTTCCGCGAGTGCCCCCCGCCG
CTGTGGAGCCACTGCTACCTGCTGCGCTTGGCCAACTACCTGGCCTAC
CACTCGGACGTGATCGAGGACGTCAGCAGCGAGGGCCTGCTCGAGTGC
CACTGCCGCTGCAACCTCTGCACGCCGCACCGCTCCCTGGCCTGCAAC
CCCCAGCTGCTGAGCGAGACCCAGATCATCGGCACCTTCGAGTTGCAA
GGGCCCGGCGATGAGGGTTCTGCCGCCAAGGGGGGTCTGAAACTCACC
CCGGGGCTGTGGACCTCGGCCTACTTGCGCAAGTTCGTGCCCGAGGAC
TACCATCCCTTCGAGATCAGGTTCTACGAGGACCAATCCCAGCCGCCC
AAGGCCGAGCTGTCGGCCTGCGTCATCACCCAGGGGGCGATCCTGGCC
CAATTGCAAGCTATCCAGAAATCCCGCCAAGAATTCTTGCTGAAAAAG
GGCCGCGGGGTCTACCTTGATCCCCAGACCGGTGAGGAGCTTAACCCC
GGCTTCCCCCAGGATGCCCCGAGGAAGCAGCAAGAAGCTGAAAGTGGA
GCTGCCGCCCGTGGAGGATTTGGAGGAAGACTGGGAGAGCAGTCAGGC
AGAGGAGGAGGAGATGGAAGACTGGGACAGCACTCAGGCAGAGGACAG
CCTGCAAGACAGTCTGGAAGACGAGGAGGAGGCAGAGGAGGTGGAAGA
AGTAGCCGCCGCCGCCAGACCGTCGTCCTCGGCGGAGAAAGCAAGCAG
CACGGATACCATCTCCGCTCCGGGTCGGGGTCCCGCTCGACCCCACAG
TAGATGGGACGAGACCGGGCGATTCCCGAACCCCACCACCCAGACCGG
TAAGAAGGAGCGGCAGGGATACAAGTCCTGGCGGGGGCACAAAAACGC
CATCGTCTCCTGCTTGCAAGCTTGCGGGGGCAACATCTCATTCACCCG
GCGCTACCTGCTCTTTCACCGCGGGGTGAACTTCCCCCGCAACATCTT
GCATTACTACCGTCACCTCCACAGCCCCTACTACTTCCAAGAAGAGGC
AGAAAAAGACAAAACCAGCAGCTAGAAAATCCACAGCGGCGGCGGCGG
CAGGTGGACTGAGGATCGCGGCGAACGAGCCGGCGCAGACCCGGGAAC
TGAGGAACCGGATCTTTCCCACCCTCTATGCCATCTTCCAGCAGAGTC
GGGGGCAGGAGCAGGAACTGAAAGTCAAGAACCGTTCTCTGCGCTCGC
TCACCCGCAGTIGTCTGTATCACAAGAGCGAAGACCAACTTCAGCGCA
CGCTTGAGGACGCCGAGGCTCTCTTCAACAAGTACTGCGCACTCACTC
TTAAAGAGTAGCCCGCGCCCGCCCACACACGGAAAAAGGCGGGAATTA
CGTCACCTGTGCACCCCCACCCAGCACCGCTATGAGCAAAGAAATTCC
CACGCCTTACATGTGGAGCTACCAGCCCCAGATGGGCCTGGCCGCCGG
CGCCGCCCAGGACTACTCCACCCGCATGAATTGGCTCAGCGCCGGGCC
CGGGATGATCTCACGGGTGAATGACATCCGCGCCCACCGAAACCAGAT
ACTCCTAGAACAGTCAGCGCTCACCGCCACGCCCCGCAATCACCTCAA
TCCGCGTAATTGGCCCGCCGCCCTAGTGTACCAGGAAATTCCCCAGCC
CACGACCGTACTACTTCCGCGAGACGCCCAGGCCGAAGTCCAGCTGAC
TAACTCAGGTGTCCAGCTGGCGGGCGGCGCCACCCTGTGTCGTCACCA
CCCCGCTCAGGGTATAAAGCGGCTGGTGATCCGGGGCAGAGGCACACA
GCTCAACGACGAGGTGGTGAGCTCTTCACTGGGTTTGCGACCTGACGG
AGTCTTCCAACTCGCCGGATCGGGAAGATCTTTTCGGGGCAACATCTC
ATTCACCCGGCGCTACCTGCTCTTTCACCGCGGGGTGAACTTCCCCCG
CAACATCTTGCATTACTACCGTCACCTCCACAGCCCCTACTACTTCCA
AGAAGAGGCAGAAAAAGACAAAACCAGCAGCTAGAAAATCCACAGCGG
CGGCGGCGGCAGGTGGACTGAGGATCGCGGCGAACGAGCCGGCGCAGA
CCCGGGAACTGAGGAACCGGATCTTTCCCACCCTCTATGCCATCTTCC
AGCAGAGTCGGGGGCAGGAGCAGGAACTGAAAGTCAAGAACCGTTCTC
TGCGCTCGCTCACCCGCAGTTGTCTGTATCACAAGAGCGAAGACCAAC
TTCAGCGCACGCTTGAGGACGCCGAGGCTCTCTTCAACAAGTACTGCG
CACTCACTCTTAAAGAGTAGCCCGCGCCCGCCCACACACGGAAAAAGG
CGGGAATTACGTCACCTGTGCACCCCCACCCAGCACCGCTATGAGCAA
AGAAATTCCCACGCCTTACATGTGGAGCTACCAGCCCCAGATGGGCCT
GGCCGCCGGCGCCGCCCAGGACTACTCCACCCGCATGAATTGGCTCAG
CGCCGGGCCCGGGATGATCTCACGGGTGAATGACATCCGCGCCCACCG
AAACCAGATACTCCTAGAACAGTCAGCGCTCACCGCCACGCCCCGCAA
TCACCTCAATCCGCGTAATTGGCCCGCCGCCCTAGTGTACCAGGAAAT
TCCCCAGCCCACGACCGTACTACTTCCGCGAGACGCCCAGGCCGAAGT
CCAGCTGACTAACTCAGGTGTCCAGCTGGCGGGCGGCGCCACCCTGTG
TCGTCACCACCCCGCTCAGGGTATAAAGCGGCTGGTGATCCGGGGCAG
AGGCACACAGCTCAACGACGAGGTGGTGAGCTCTTCACTGGGTTTGCG
ACCTGACGGAGTCTTCCAACTCGCCGGATCGGGAAGATCTTCCTTCAC
GCCTCGTCAGGCCGTGCTGACTTTGGAGAGTTCTTCCTCGCAACCTCG
CTCGGGCGGCATCGGCACTCTCCAGTTTGTGGAGGAGTTCACTCCCTC
GGTCTACTTCAACCCCTTCTCCGGCTCCCCCGGCCACTATCCGGACGA
GTTCATCCCGAACTTCGATGCCATCAGCGAATCGGTAGACGGCTACGA
TTGAATGTCCCATGGTGGCGCGGCTGACCTAGCTCGGCTTCGACACCT
GGACCACTGCCGCCGCTTTCGCTGCTTCGCTCGGGACCTCGCCGAGTT
TACCTACTTTGAGCTGTCCGAGGAGCACCCTCAGGGCCCGGCCCACGG
AGTGCGGATCGTCGTCGAAGGGGGCCTAGACTCCCACCTGCTTCGTAT
CTTCAGCCAGCGCCCGATCCTGGTCCAGCGCCAACAGGGCAACACCCT
CCTGACCCTTTACTGCATCTGCAACCACCCCGGCCTGCACGAAAGTCT
TTGTTGTCTGCTGTGTACTGAGTATAATAAAAGCTGAGATCAGCGACT
ACTCCGGACTCGATTGTGTTCCAGCAGTCTGGCGATACCAAGGGTTGC
ATCCACTGCTCCTGCGACTCCCCCGAGTGCGTTCACACCCTCATCAAG
ACCCTATGCGGCCTCCGCGACCTCCTCCCCATGAACTAATCAACTAAC
CCCTTACCCCATTACCCATCCAGTAAAAAAAATAAAGATTAAAGAGAC
GATGATTTTGAATTACTAGTTATTAATAGTAATCAATTACGGGGTCAT
TAGTTCATAGCCCATATATGGAGTTCCGCGTTACATAACTTACGGTAA
ATGGCCCGCCTGGCTGACCGCCCAACGACCCCCGCCCATTGACGTCAA
TAATGACGTATGTTCCCATAGTAACGCCAATAGGGACTTTCCATTGAC
GTCAATGGGTGGAGTATTTACGGTAAACTGCCCACTTGGCAGTACATC
AAGTGTATCATATGCCAAGTACGCCCCCTATTGACGTCAATGACGGTA
AATGGCCCGCCTGGCATTATGCCCAGTACATGACCTTATGGGACTTTC
CTACTTGGCAGTACATCTACGTATTAGTCATCGCTATTACCATGGTGA
TGCGGTTTTGGCAGTACATCAATGGGCGTGGATAGCGGTTTGACTCAC
GGGGATTTCCAAGTCTCCACCCCATTGACGTCAATGGGAGTTTGTTTT
GGCACCAAAATCAACGGGACTTTCCAAAATGTCGTAACAACTCCGCCC
CATTGACGCAAATGGGCGGTAGGCGTGTACGGTGGGAGGTCTATATAA
GCAGAGCTCACTGTCTTCCGGATCGCTGTCCAGGAGCGCCAGCTGTTG
GGCTCGCGGTTGAGAAGGAACTCTTCGCGGTCCTTCCAGTACTCTTCA
AGGGGGAACCCGTCCTGGTCGGCACGGGACTCCGCGCAAGGACCTAAG
CGTCTCCAGATCCACGGGATCTGAAAACCGTTGAACGAAGGCTTCGAG
CCAGTCGCAGTCGCAAGTCTAGAGCCACCATGTTCGTCTTCCTGGTCC
TGCTGCCCCTGGTCTCATCTCAGTGCGTGAATCTGACTACAAGAACTC
AGCTGCCTCCCGCCTACACCAATTCCTTCACCCGGGGCGTGTACTATC
CTGACAAGGTGTTTAGAAGCTCCGTGCTGCACTCTACACAGGATCTGT
TTCTGCCATTCTTTAGCAACGTGACCTGGTTCCACGCCATCCACGTGA
GCGGCACCAATGGCACAAAGCGGTTCGACAATCCCGTGCTGCCTTTTA
ACGATGGCGTGTACTTCGCCTCTACCGAGAAGAGCAACATCATCAGAG
GCTGGATCTTTGGCACCACACTGGACTCCAAGACACAGTCTCTGCTGA
TCGTGAACAATGCCACCAACGTGGTCATCAAGGTGTGCGAGTTCCAGT
TTTGTAATGATCCCTTCCTGGGCGTGTACTATCACAAGAACAATAAGA
GCTGGATGGAGTCCGAGTTTAGAGTGTATTCTAGCGCCAACAATTGCA
CATTTGAGTACGTGTCCCAGCCTTTCCTGATGGACCTGGAGGGCAAGC
AGGGCAATTTCAAGAACCTGAGGGAGTTCGTGTTTAAGAATATCGATG
GCTACTTCAAGATCTACTCTAAGCACACCCCCATCAACCTGGTGCGCG
ACCTGCCTCAGGGCTTCAGCGCCCTGGAGCCACTGGTGGATCTGCCTA
TCGGCATCAACATCACCCGGTTTCAGACACTGCTGGCCCTGCACAGAA
GCTACCTGACACCCGGCGACTCCTCTAGCGGATGGACCGCAGGAGCAG
CAGCCTACTATGTGGGCTATCTGCAGCCTAGGACCTTCCTGCTGAAGT
ACAACGAGAATGGCACCATCACAGACGCAGTGGATTGCGCCCTGGACC
CCCTGAGCGAGACAAAGTGTACACTGAAGTCCTTTACCGTGGAGAAGG
GCATCTATCAGACATCCAATTTCAGGGTGCAGCCAACCGAGTCTATCG
TGCGCTTTCCTAATATCACAAACCTGTGCCCATTTGGCGAGGTGTTCA
ACGCAACCAGGTTCGCAAGCGTGTACGCATGGAATAGGAAGCGCATCT
CTAACTGCGTGGCCGACTATAGCGTGCTGTACAACTCCGCCTCTTTCA
GCACCTTTAAGTGCTATGGCGTGTCCCCCACAAAGCTGAATGACCTGT
GCTTTACCAACGTGTACGCCGATTCTTTCGTGATCAGGGGCGACGAGG
TGCGCCAGATCGCACCTGGACAGACAGGCAAGATCGCCGACTACAATT
ATAAGCTGCCAGACGATTTCACCGGCTGCGTGATCGCCTGGAACAGCA
ACAATCTGGATTCCAAAGTGGGCGGCAACTACAATTATCTGTACCGGC
TGTTTAGAAAGAGCAATCTGAAGCCCTTCGAGAGGGACATCTCTACAG
AGATCTACCAGGCCGGCAGCACCCCTTGCAATGGCGTGGAGGGCTTTA
ACTGTTATTTCCCACTGCAGTCCTACGGCTTCCAGCCCACAAACGGCG
TGGGCTATCAGCCTTACCGCGTGGTGGTGCTGAGCTTTGAGCTGCTGC
ACGCACCAGCAACAGTGTGCGGACCCAAGAAGTCCACCAATCTGGTGA
AGAACAAGTGCGTGAACTTCAACTTCAACGGCCTGACCGGAACAGGCG
TGCTGACCGAGTCCAACAAGAAGTTCCTGCCATTTCAGCAGTTCGGCA
GGGACATCGCAGATACCACAGACGCCGTGCGCGACCCACAGACCCTGG
AGATCCTGGATATCACACCCTGCTCTTTCGGCGGCGTGAGCGTGATCA
CACCAGGAACCAATACAAGCAACCAGGTGGCCGTGCTGTATCAGGACG
TGAATTGTACCGAGGTGCCTGTGGCCATCCACGCCGATCAGCTGACCC
CAACATGGCGGGTGTACAGCACCGGCTCCAACGTGTTCCAGACAAGAG
CAGGATGCCTGATCGGAGCAGAGCACGTGAACAATTCCTATGAGTGCG
ACATCCCAATCGGCGCCGGCATCTGTGCCTCTTACCAGACCCAGACAA
ACTCTCCAAGGAGAGCACGGAGCGTGGCATCCCAGTCTATCATCGCCT
ATACCATGTCCCTGGGCGCCGAGAATTCTGTGGCCTACTCTAACAATA
GCATCGCCATCCCAACCAACTTCACAATCTCTGTGACCACAGAGATCC
TGCCCGTGTCCATGACCAAGACATCTGTGGACTGCACAATGTATATCT
GTGGCGATTCTACCGAGTGCAGCAACCTGCTGCTGCAGTACGGCAGCT
TTTGTACCCAGCTGAATAGAGCCCTGACAGGCATCGCCGTGGAGCAGG
ATAAGAACACACAGGAGGTGTTCGCCCAGGTGAAGCAGATCTACAAGA
CCCCCCCTATCAAGGACTTTGGCGGCTTCAATTTTTCCCAGATCCTGC
CTGATCCATCCAAGCCTTCTAAGCGGAGCTTTATCGAGGACCTGCTGT
TCAACAAGGTGACCCTGGCCGATGCCGGCTTCATCAAGCAGTATGGCG
ATTGCCTGGGCGACATCGCAGCACGGGACCTGATCTGTGCCCAGAAGT
TTAATGGCCTGACCGTGCTGCCACCCCTGCTGACAGATGAGATGATCG
CACAGTACACAAGCGCCCTGCTGGCAGGAACCATCACATCCGGATGGA
CCTTCGGCGCAGGAGCCGCCCTGCAGATCCCCTTTGCCATGCAGATGG
CCTATAGGTTCAACGGCATCGGCGTGACCCAGAATGTGCTGTACGAGA
ACCAGAAGCTGATCGCCAATCAGTTTAACTCCGCCATCGGCAAGATCC
AGGACAGCCTGTCCTCTACAGCCTCCGCCCTGGGCAAGCTGCAGGATG
TGGTGAATCAGAACGCCCAGGCCCTGAATACCCTGGTGAAGCAGCTGA
GCTCCAACTTCGGCGCCATCTCTAGCGTGCTGAATGATATCCTGAGCC
GGCTGGACAAGGTGGAGGCAGAGGTGCAGATCGACCGGCTGATCACAG
GCAGACTGCAGTCTCTGCAGACCTATGTGACACAGCAGCTGATCAGGG
CAGCAGAGATCAGGGCAAGCGCCAATCTGGCAGCAACCAAGATGTCCG
AGTGCGTGCTGGGCCAGTCTAAGAGAGTGGACTTTTGTGGCAAGGGCT
ATCACCTGATGTCCTTCCCTCAGTCTGCCCCACACGGCGTGGTGTTTC
TGCACGTGACCTACGTGCCCGCCCAGGAGAAGAACTTCACCACAGCCC
CTGCCATCTGCCACGATGGCAAGGCCCACTTTCCAAGGGAGGGCGTGT
TCGTGTCCAACGGCACCCACTGGTTTGTGACACAGCGCAATTTCTACG
AGCCCCAGATCATCACCACAGACAATACCTTCGTGAGCGGCAACTGTG
ACGTGGTCATCGGCATCGTGAACAATACCGTGTATGATCCACTGCAGC
CCGAGCTGGACAGCTTTAAGGAGGAGCTGGATAAGTACTTCAAGAATC
ACACCTCCCCTGACGTGGATCTGGGCGACATCAGCGGCATCAATGCCT
CCGTGGTGAACATCCAGAAGGAGATCGACCGCCTGAACGAGGTGGCCA
AGAATCTGAACGAGAGCCTGATCGATCTGCAGGAGCTGGGCAAGTATG
AGCAGTACATCAAGTGGCCATGGTACATCTGGCTGGGCTTCATCGCCG
GCCTGATCGCCATCGTGATGGTGACCATCATGCTGTGCTGTATGACAT
CCTGCTGTTCTTGCCTGAAGGGCTGCTGTAGCTGTGGCTCCTGCTGTA
AGTTTGATGAGGACGATTCCGAACCCGTGCTGAAGGGAGTGAAGCTGC
ATTACACCTGAGGATCCCTCGAGCTGTGCCTTCTAGTTGCCAGCCATC
TGTTGTTTGCCCCTCCCCCGTGCCTTCCTTGACCCTGGAAGGTGCCAC
TCCCACTGTCCTTTCCTAATAAAATGAGGAAATTGCATCGCATTGTCT
GAGTAGGTGTCATTCTATTCTGGGGGGTGGGGTGGGGCAGGACAGCAA
GGGGGAGGATTGGGAAGACAATAGCAGGCATGCTGGGGATGCGGTGGG
CTCTATGGTGATCAATAAAGAATCACTTACTTGAAATCTGAAACCAGG
TCTCTGTCCATGTTTTCTGTCAGCAGCACTTCGCTCCCCTCTTCCCAG
CTCTGGTACTGCAGGCCCCGGCGGGCTGCAAACTTCCTCCACACTCTG
AAGGGGATGTCAAATTCCTCCTGTCCCTCAATCTTCATTTTTTATTTC
TATTAGATGTCCAAAAAGCGCGCGCGGGTGGATGATGGCTTCGACCCC
GTGTATCCCTACGATGCAGACAACGCACCGACCGTGCCCTTCATCAAC
CCTCCCTTCGTCTCTTCAGATGGATTCCAAGAAAAGCCCCTGGGGGTG
TTGTCCCTTAGGCTGGCCGACCCTGTCACCACCAAGAATGGGGAAATT
ACCCTCAAGCTGGGGGAGGGGGTGGACCTTGACGACTCGGGAAAACTC
ATTGCAAACACAGTAAACAAGGCCATTGCCCCTCTCAGTTTTTCCAAC
AACACCATTTCCCTTAACATGGATACCCCTTTATACACCAAAGATGGA
AAACTATCCTTACAAGTTTCTCCACCATTAAGTATATTAAAATCAACA
ATTTTGAATACATTAGCTCTAGCTTTTGGCTCAGGTTTAGGACTCAGT
GGCAGCGCCCTGGCAGTACAGTTAGCCTCTCCACTTACATTTGATGAT
AAAGGGAATATAAAGATTACCCTAAACAGGGGATTGCATGTTACAACA
GGAGATGCAATTGAAAGCAACATCAGTTGGGCTAAAGGTATAAAATTT
GAAGATGGTGCCATAGCTACAAACATTGGTAAGGGGCTAGAGTTCGGA
ACCAGTAGTACAGAAACAGGAGTTAATAATGCTTATCCAATCCAAGTT
AAACTTGGCTCTGGTCTCAGCTTTGACAGCACAGGAGCCATAATGGCT
GGCAATAAAGACTATGATAAATTAACTTTGTGGACAACGCCTGACCCA
TCACCAAACTGTCAAATACTTGCAGAAAATGATGCAAAACTAACACTT
TGCTTAACTAAGTGTGACAGTCAAATACTGGCCACTGTATCAGTTTTG
GTTGTTAGAAGTGGAAACTTAAACCCAATTACTGGCACAGTAAGCAGT
GCTCAAGTTTTTCTACGTTTTGATGCAAATGGTGTTCTTTTAACAGAA
CACTCTACACTAAAAAAATACTGGGGCTACAAGCAAGGAGATAGCATA
GATGGCACTCCATACACCAATGCTGTTGGTTTTATGCCAAATTCAACA
GCTTATCCAAAGACCCAAAGTTCTACTACTAAAAATAATATAGTGGGT
CAAGTATACATGAATGGAGATGTTTCAAAACCCATGCTTCTTACTATA
ACTCTTAATGGTACTGATGACACCACCAGTGCATACTCAATGTCATTT
TCATACACCTGGACTAACGGAAGCTATATCGGAGCAACATTTGGAGCT
AACTCATACACCTTCTCCTACATAGCCCAACAATAATCCCACCCTGCA
TGCCAACCCACCTTTTCCCTCTATTTATAAATGGAAACTGAAACAAAA
ATAAAGTTCAAGTGTTTTATTGATTCAACAGTTTTTCACAGGATTCGA
GTAGTTATTTTCCCTCCACCCTCCCATCTCATGGAATACACTATCCTC
TCCCCACGCACAGCCTTAAACATCTGAATGCTATTGGTAATGGACATG
GTTTTGATCTCCACATTCCACACAGTTTCAGAGCGAGACAGTCTCGGG
TCGGTCAAGGAGATGAAACCCTCCGGGCACTCCTGCATCTGCACCTCA
CAGTTCAACAGCTGAGGGCTGTCCTCGGTGATTGGAATCACAGTTATC
TGGAATAAGAGCGATGAGAATCATAATCCGCAAACGGGATCGGGCGGT
TGTGGCGCATCAGGCCCCGCAGCAGTCGCTGTCTGCGCCGCTCCGTCA
AGCTGCTACTCAAGGGGTCCGGGTCCAGGGACTCCCTGCGCATGATGC
CAATGGCCCTGAGCATCAGTCGCCTGGTACGGCGGGCGCAGCAGCGGA
TGCGGATCTCACTCAGGTCGGAGCAGTACGTGCAGCACAGCACCACCA
AGTTGTTCAACAGTCCATAGTTCAACGTGCTCCAGCCAAAACTCATTT
GTGGAACTATGCTGCCCACATGTCCATCGTACCAGATCCTGATGTAAA
TCAGGTGGCGTCCCCTCCAGAACACACTGCCCATGTACATGATCTCCT
TGGGCATGTGCAGGTTCACCACCTCCCGGTACCACATCACCCGCTGGT
TGAACATGCAGCCCTGGATAATTCTGCGGAACCAGATGGCAAGTACCG
TCCCGCCCGCCATGCAGCGCAGGGACCCCGGGTTCTGGCAATGGCAGT
GGATCACCCACCGCTCGCGACCGTGGATCAACTGGGAACTAAACAAGT
CTATGTTGGCACAGCACAGGCACACGCTCATGCATGTCTTCAGCACTC
TCAATTCCTCGGGGGTCAGGACCATATCCCAGGGCACAGGGAACTCTT
GCAGGACAGTGAACCCGGCCGAACAGGGCAATCCTCGCACGGAACTTA
CATTGTGCATGGACAGGGTATCGCAATCAGGCAGCACCGGATGATCCT
CCACCAGAGAAGCGCGGCTCTCGGTCTCCTCACAGCGAGGTAAGGTGG
CCGGCGGTTGGTACGGATGATGGCGAGATAACGCTAATCGTGTTCTGG
ATCGTGTCATGATGGAGCTGTTTCCGGACATTTTCGTATTTCACAAAG
CAGAACCTGGTCCGGGCACTGCACACCGCTCGTCGGCGACGGTCTCGG
CGCTTCGAGCGCTCAATGTTGAAGTTATAGAACAGCCACTCCCTCAGA
ACGTGCAGTATCTCCTGAGCCTCTTGGGTGATGAAAATCCCATCCGCC
CTGATGGCTCTGATTACATCAACCACGGTGGAATGGGCCAAACCCAGC
CAGATGATGCAATTTTGTTGGGTTTCGGTGACGGCGGGGGAGGGAAGA
ACAGGAAGAACCATGATTAACTTTATTCCAAACGGTCTCGGAACACTT
CAAAATGCAGGTCCCGGAGGTGGCACCTCTCGCCCCCACTGTGTTGGT
GGAAAATAACAGCCAGGTCAAAGGTAACACGGTTCTCGAGATGTTCCA
CGGTGGCTTCCAGCAAAGCCTCCACGCGCACATCCAGAAACAAGAGGA
CAGCGAAAGCGGGAGCGTTTTCTAATTCCTCAATCATCATATTACACT
CCTGCACCATGCCTAGATAATTTTCATTTTTCCAGCCTTGAATGATTC
GTATTAGTTCCTGAGGTAAATCCAAGCCAGCCATGATAAAAAGCTCGC
GCAGAGCGCCCTCCACCGGCATTCTTAAGCACACCCTCATAATTCCAA
CAGATTCTGCTCCTGGTTCACCTGTAGTAGATTAACAAGTGGAATATC
AATTGCTCTGCCGCAATCCCTAAGCTCCTCCCTTAGCAGTAACTGTAT
GTACTCATTCATATCTTCTCCGAAATTTTTAGCCATAGGACCACCAGG
AACAAGAGAAGGGCAAGCCACATTACAGATAAAGCGAAGTCCTCCCCA
GTGAGCATTGCCAAATGTAAGATTGAAATAAGCATGCTGGCTAGACCC
GGTGATATCTTCCAGATAACTGGACAGAAAATCAGGCAAGCAATTTTT
AAGAAAATTAACAAAAGAAAAGTCGTCTAGGTGCACGTTTAGAGCCTC
AGGAACAACGATGGAATAAGTGCAAGGAGTACGTTCCAGCATGGTTAG
TGTTTTTGGTGATCTGTAGAACAAAAAATAAACATGCAATATTAAACC
ATGCTAGCCTGGCGAACAGGTGGATAAATCACTCTTTCCAACACCAGG
CAGGCTACAGGGTCTCCGGCGCGACCATTGTAGAAGCTGACATTATGA
TTAAAAAGCATCACCGACAGACCTTCCCGGTGGCCGGCATGGATGATT
CGAGAAGAAGCATACACTCCGGGAACATTGGCGTCCGTGAGTGAAAAA
AAGCGACCTATAAAGCCTTGAGGCACTACAATGCTTAATCTTAATTCC
AGCAAAGCGACCCCATGCGGATGAAGCACAAAATTGGCAGGTGCGTAA
AAAATGTAATTACTCCCCTTCTGCACAGGCAGCAAAGCCCCCGCTCCC
TCCAGAAACACATACAAAACCTGAGCGTCCATAGCTTACCGAGCACGG
CAGGCGCAAGAGTCAGAGAAAAAGCTGAGCTCTAACCTAACTGCCCGC
TTCTGTACTCAATATATAGCCCTAACCTCACTGACGTAAAGGCCAAGG
TCTAAAAATACCCGCCAACACGCCCAGAAACCGGTGACACACTAAAAA
AATACGTGCACTTCCTCAAACGCCCAAACTGGCGTCATTTCCGGTTTC
CCACGCTACGTCACCTCTCAACGACTTTCAAATTCCGTCGACCGTTAA
ACACATCAGTTACCCCGCCCCTAACGAACGCCGCTGTCACAGCCAATC
AGCGCGCCCCATCCCCAAATTTTCACGCCTTATTTGCATATTAACTCA
CACAAAAAAAATAAGGTATATTATTGATGATGAAGCTTTTAAT
SEQ ID NO: 2 is the amino acid sequence
of a wild-type SARS-CoV-2 (Wuhan strain)
spike protein deposited under GenBank
Accession No. YP_009724390.1.
MFVFLVLLPLVSSQCVNLTTRTQLPPAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNERVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLERKSNLKPFERDISTEIYQAGSTPC
NGVEGENCYFPLQSYGFQPTNGVGYQPYRVVVLSFELLHAPATVCGPK
KSTNLVKNKCVNFNFNGLTGTGVLTESNKKELPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDEGGFNFSQILPDPSKPSKRS
FIEDLLFNKVTLADAGFIKQYGDCLGDIAARDLICAQKENGLTVLPPL
LTDEMIAQYTSALLAGTITSGWTFGAGAALQIPFAMQMAYRENGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT
SEQ ID NO: 3 is the amino acid sequence
of a stabilized SARS-CoV-2 spike protein
with a double proline substitution (nCoV-PP).
MFVFLVLLPLVSSQCVNLTTRTQLPPAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPK
KSTNLVKNKCVNFNFNGLTGTGVLTESNKKELPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKENGLTVLPPL
LTDEMIAQYTSALLAGTITSGWTFGAGAALQIPFAMQMAYRENGIGVT
QNVLYENQKLIANQFNSAIGKIQDSLSSTASALGKLQDVVNQNAQALN
TLVKQLSSNFGAISSVLNDILSRLDPPEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT
SEQ ID NO: 4 is the amino acid sequence
of a tail-truncated SARS-CoV-2 spike
protein (nCoV-TT).
MFVFLVLLPLVSSQCVNLTTRTQLPPAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNERVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPK
KSTNLVKNKCVNFNFNGLTGTGVLTESNKKELPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDEGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRENGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
S
SEQ ID NO: 5 is the amino acid sequence
of a SARS-CoV-2 spike protein lacking
the C- terminal endocytosis motif
(nCoV-noEndo).
MFVFLVLLPLVSSQCVNLTTRTQLPPAYTNSFTRGVYYPDKVFRSSVL
HSTQDLFLPFFSNVTWFHAIHVSGINGTKREDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNERVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPK
KSTNLVKNKCVNFNFNGLTGTGVLTESNKKELPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKENGLTVLPPL
LTDEMIAQYTSALLAGTITSGWTFGAGAALQIPFAMQMAYRENGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGV
SEQ ID NO: 6 is a nucleic acid sequence
encoding a SARS-CoV-2 spike protein.
ATGTTTGTTTTTCTTGTTTTATTGCCACTAGTCTCTAGTCAGTGTGTT
AATCTTACAACCAGAACTCAATTACCCCCTGCATACACTAATTCTTTC
ACACGTGGTGTTTATTACCCTGACAAAGTTTTCAGATCCTCAGTTTTA
CATTCAACTCAGGACTTGTTCTTACCTTTCTTTTCCAATGTTACTTGG
TTCCATGCTATACATGTCTCTGGGACCAATGGTACTAAGAGGTTTGAT
AACCCTGTCCTACCATTTAATGATGGTGTTTATTTTGCTTCCACTGAG
AAGTCTAACATAATAAGAGGCTGGATTTTTGGTACTACTTTAGATTCG
AAGACCCAGTCCCTACTTATTGTTAATAACGCTACTAATGTTGTTATT
AAAGTCTGTGAATTTCAATTTTGTAATGATCCATTTTTGGGTGTTTAT
TACCACAAAAACAACAAAAGTTGGATGGAAAGTGAGTTCAGAGTTTAT
TCTAGTGCGAATAATTGCACTTTTGAATATGTCTCTCAGCCTTTTCTT
ATGGACCTTGAAGGAAAACAGGGTAATTTCAAAAATCTTAGGGAATTT
GTGTTTAAGAATATTGATGGTTATTTTAAAATATATTCTAAGCACACG
CCTATTAATTTAGTGCGTGATCTCCCTCAGGGTTTTTCGGCTTTAGAA
CCATTGGTAGATTTGCCAATAGGTATTAACATCACTAGGTTTCAAACT
TTACTTGCTTTACATAGAAGTTATTTGACTCCTGGTGATTCTTCTTCA
GGTTGGACAGCTGGTGCTGCAGCTTATTATGTGGGTTATCTTCAACCT
AGGACTTTTCTATTAAAATATAATGAAAATGGAACCATTACAGATGCT
GTAGACTGTGCACTTGACCCTCTCTCAGAAACAAAGTGTACGTTGAAA
TCCTTCACTGTAGAAAAAGGAATCTATCAAACTTCTAACTTTAGAGTC
CAACCAACAGAATCTATTGTTAGATTTCCTAATATTACAAACTTGTGC
CCTTTTGGTGAAGTTTTTAACGCCACCAGATTTGCATCTGTTTATGCT
TGGAACAGGAAGAGAATCAGCAACTGTGTTGCTGATTATTCTGTCCTA
TATAATTCCGCATCATTTTCCACTTTTAAGTGTTATGGAGTGTCTCCT
ACTAAATTAAATGATCTCTGCTTTACTAATGTCTATGCAGATTCATTT
GTAATTAGAGGTGATGAAGTCAGACAAATCGCTCCAGGGCAAACTGGA
AAGATTGCTGATTATAATTATAAATTACCAGATGATTTTACAGGCTGC
GTTATAGCTTGGAATTCTAACAATCTTGATTCTAAGGTTGGTGGTAAT
TATAATTACCTGTATAGATTGTTTAGGAAGTCTAATCTCAAACCTTTT
GAGAGAGATATTTCAACTGAAATCTATCAGGCCGGTAGCACACCTTGT
AATGGTGTTGAAGGTTTTAATTGTTACTTTCCTTTACAATCATATGGT
TTCCAACCCACTAATGGTGTTGGTTACCAACCATACAGAGTAGTAGTA
CTTTCTTTTGAACTTCTACATGCACCAGCAACTGTTTGTGGACCTAAA
AAGTCTACTAATTTGGTTAAAAACAAATGTGTCAATTTCAACTTCAAT
GGTTTAACAGGCACAGGTGTTCTTACTGAGTCTAACAAAAAGTTTCTG
CCTTTCCAACAATTTGGCAGAGACATTGCTGACACTACTGATGCTGTC
CGTGATCCACAGACACTTGAGATTCTTGACATTACACCATGTTCTTTT
GGTGGTGTCAGTGTTATAACACCAGGAACAAATACTTCTAACCAGGTT
GCTGTTCTTTATCAGGATGTTAACTGCACAGAAGTCCCTGTTGCTATT
CATGCAGATCAACTTACTCCTACTTGGCGTGTTTATTCTACAGGTTCT
AATGTTTTTCAAACACGTGCAGGCTGTTTAATAGGGGCTGAACATGTC
AACAACTCATATGAGTGTGACATACCCATTGGTGCAGGTATATGCGCT
AGTTATCAGACTCAGACTAATTCTCCTCGGCGGGCACGTAGTGTAGCT
AGTCAATCCATCATTGCCTACACTATGTCACTTGGTGCAGAAAATTCA
GTTGCTTACTCTAATAACTCTATTGCCATACCCACAAATTTTACTATT
AGTGTTACCACAGAAATTCTACCAGTGTCTATGACCAAGACATCAGTA
GATTGTACAATGTACATTTGTGGTGATTCAACTGAATGCAGCAATCTT
TTGTTGCAATATGGCAGTTTTTGTACACAATTAAACCGTGCTTTAACT
GGAATAGCTGTTGAACAAGACAAAAACACCCAAGAAGTTTTTGCACAA
GTCAAACAAATTTACAAAACACCACCAATTAAAGATTTTGGTGGTTTT
AATTTTTCACAAATATTACCAGATCCATCAAAACCAAGCAAGAGGTCA
TTTATTGAAGATCTACTTTTCAACAAAGTGACACTTGCAGATGCTGGC
TTCATCAAACAATATGGTGATTGCCTTGGTGATATTGCTGCTAGAGAC
CTCATTTGTGCACAAAAGTTTAACGGCCTTACTGTTTTGCCACCTTTG
CTCACAGATGAAATGATTGCTCAATACACTTCTGCACTGTTAGCGGGT
ACAATCACTTCTGGTTGGACCTTTGGTGCAGGTGCTGCATTACAAATA
CCATTTGCTATGCAAATGGCTTATAGGTTTAATGGTATTGGAGTTACA
CAGAATGTTCTCTATGAGAACCAAAAATTGATTGCCAACCAATTTAAT
AGTGCTATTGGCAAAATTCAAGACTCACTTTCTTCCACAGCAAGTGCA
CTTGGAAAACTTCAAGATGTGGTCAACCAAAATGCACAAGCTTTAAAC
ACGCTTGTTAAACAACTTAGCTCCAATTTTGGTGCAATTTCAAGTGTT
TTAAATGATATCCTTTCACGTCTTGACAAAGTTGAGGCTGAAGTGCAA
ATTGATAGGTTGATCACAGGCAGACTTCAAAGTTTGCAGACATATGTG
ACTCAACAATTAATTAGAGCTGCAGAAATCAGAGCTTCTGCTAATCTT
GCTGCTACTAAAATGTCAGAGTGTGTACTTGGACAATCAAAAAGAGTT
GATTTTTGTGGAAAGGGCTATCATCTTATGTCCTTCCCTCAGTCAGCA
CCTCATGGTGTAGTCTTCTTGCATGTGACTTATGTCCCTGCACAAGAA
AAGAACTTCACAACTGCTCCTGCCATTTGTCATGATGGAAAAGCACAC
TTTCCTCGTGAAGGTGTCTTTGTTTCAAATGGCACACACTGGTTTGTA
ACACAAAGGAATTTTTATGAACCACAAATCATTACTACAGACAACACA
TTTGTGTCTGGTAACTGTGATGTTGTAATAGGAATTGTCAACAACACA
GTTTATGATCCTTTGCAACCTGAATTAGACTCATTCAAGGAGGAGTTA
GATAAATATTTTAAGAATCATACATCACCAGATGTTGATTTAGGTGAC
ATCTCTGGCATTAATGCTTCAGTTGTAAACATTCAAAAAGAAATTGAC
CGCCTCAATGAGGTTGCCAAGAATTTAAATGAATCTCTCATCGATCTC
CAAGAACTTGGAAAGTATGAGCAGTATATAAAATGGCCATGGTACATT
TGGCTAGGTTTTATAGCTGGCTTGATTGCCATAGTAATGGTGACAATT
ATGCTTTGCTGTATGACCAGTTGCTGTAGTTGTCTCAAGGGCTGTTGT
TCTTGTGGATCCTGCTGCAAATTTGATGAAGACGACTCTGAGCCAGTG
CTCAAAGGAGTCAAATTACATTACACATAA
SEQ ID NO: 7 is the amino acid sequence
of a stabilized SARS-CoV-2 beta variant spike
protein with a double proline substitution.
MFVFLVLLPLVSSQCVNFTTRTQLPPAYTNSFTRGVYYPDKVFRSSVL
HSTQDLFLPFFSNVTWFHAIHVSGTNGTKRFANPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRGLPQGFSALEPLVDLPIGINITRFQT
LHISYLTPGDSSSGWTAGAAAYYVGYLQPRTFLLKYNENGTITDAVDC
ALDPLSETKCTLKSFTVEKGIYQTSNERVQPTESIVRFPNITNLCPFG
EVENATRFASVYAWNRKRISNCVADYSVLYNSASFSTFKCYGVSPTKL
NDLCFTNVYADSFVIRGDEVRQIAPGQTGNIADYNYKLPDDFTGCVIA
WNSNNLDSKVGGNYNYLYRLFRKSNLKPFERDISTEIYQAGSTPCNGV
KGFNCYFPLQSYGFQPTYGVGYQPYRVVVLSFELLHAPATVCGPKKST
NLVKNKCVNFNFNGLTGTGVLTESNKKELPFQQFGRDIADTTDAVRDP
QTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQ
TQTNSPRRARSVASQSIIAYTMSLGVENSVAYSNNSIAIPTNFTISVT
TEILPVSMTKTSVDCTMYICGDSTECSNLLLQYGSFCTQLNRALTGIA
VEQDKNTQEVFAQVKQIYKTPPIKDEGGENFSQILPDPSKPSKRSFIE
DLLFNKVTLADAGFIKQYGDCLGDIAARDLICAQKENGLTVLPPLLTD
EMIAQYTSALLAGTITSGWTFGAGAALQIPFAMQMAYRENGIGVTQNV
LYENQKLIANQFNSAIGKIQDSLSSTASALGKLQDVVNQNAQALNTLV
KQLSSNFGAISSVLNDILSRLDPPEAEVQIDRLITGRLQSLQTYVTQQ
LIRAAEIRASANLAATKMSECVLGQSKRVDFCGKGYHLMSFPQSAPHG
VVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKY
FKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCG
SCCKFDEDDSEPVLKGVKLHYT
SEQ ID NO: 8 is the amino acid sequence
of a stabilized, double proline-substituted,
chimeric SARS-CoV-2 spike protein comprising
the RBD of the beta variant and remaining
sequence from the Wuhan strain.
MFVFLVLLPLVSSQCVNLTTRTQLPPAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNERVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGNIADYNYKLPDDFTGC
VIAWNSNNLDSKVGGNYNYLYRLFRKSNLKPFERDISTEIYQAGSTPC
NGVKGFNCYFPLQSYGFQPTYGVGYQPYRVVVLSFELLHAPATVCGPK
KSTNLVKNKCVNFNFNGLTGTGVLTESNKKELPFQQFGRDIADTTDAV
RDPQTLEILDITPCSFGGVSVITPGTNTSNQVAVLYQGVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKENGLTVLPPL
LTDEMIAQYTSALLAGTITSGWTFGAGAALQIPFAMQMAYRENGIGVT
QNVLYENQKLIANQFNSAIGKIQDSLSSTASALGKLQDVVNQNAQALN
TLVKQLSSNFGAISSVLNDILSRLDPPEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT
SEQ ID NO: 9 is the amino acid sequence
of a stabilized SARS-CoV-2 delta variant
spike protein with a double proline
substitution.
MFVFLVLLPLVSSQCVNLTTTTQLPPAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNDPFLDVY
YHKNNKSWMKSEFRVYSSANNCTFEYVSQPFLMDLEGKQGNFKNLREF
VFKNIDGYFKIYSKHTPINLVRDLPHGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYRYRLFRKSNLKPFERDISTEIYQAGSTPC
NGVQGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQGVNCTEVPVAI
HADQLTPTWRVYSTGSNVFQTRAGCLIGAEHVNNSYECDIPIGAGICA
SYQTQTNSRRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLENKVTLADAGFIKQYGDCLGDIAARDLICAQKENGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDPPEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTDWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT
SEQ ID NO: 10 is the amino acid sequence
of a stabilized SARS-CoV-2 gamma variant
spike protein with a double proline
substitution.
MFVFLVLLPLVSSQCVNFTNRTQLPSAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNYPFLGVY
YHKNNKSWMESEFRVYSSANNCTFEYVSQPFLMDLEGKQGNFKNLSEF
VFKNIDGYFKIYSKHTPINLVRDLPQGFSALEPLVDLPIGINITRFQT
LLALHRSYLTPGDSSSGWTAGAAAYYVGYLQPRTELLKYNENGTITDA
VDCALDPLSETKCTLKSFTVEKGIYQTSNERVQPTESIVRFPNITNLC
PFGEVENATRFASVYAWNRKRISNCVADYSVLYNSASFSTFKCYGVSP
TKLNDLCFTNVYADSEVIRGDEVRQIAPGQTGTIADYNYKLPDDFTGC
VIAWNSNNLDSKVGGNYNYLYRLERKSNLKPFERDISTEIYQAGSTPC
NGVKGFNCYFPLQSYGFQPTYGVGYQPYRVVVLSFELLHAPATVCGPK
KSTNLVKNKCVNFNFNGLTGTGVLTESNKKELPFQQFGRDIADTTDAV
RDPQTLEILDITPCSFGGVSVITPGTNTSNQVAVLYQGVNCTEVPVAI
HADQLTPTWRVYSTGSNVFQTRAGCLIGAEYVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKENGLTVLPPL
LTDEMIAQYTSALLAGTITSGWTFGAGAALQIPFAMQMAYRENGIGVT
QNVLYENQKLIANQFNSAIGKIQDSLSSTASALGKLQDVVNQNAQALN
TLVKQLSSNFGAISSVLNDILSRLDPPEAEVQIDRLITGRLQSLQTYV
TQQLIRAAEIRASANLAAIKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASFVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT
SEQ ID NO: 11 is the amino acid sequence
of a stabilized SARS-CoV-2 delta plus
variant spike protein with a double
proline substitution.
MFVFLVLLPLVSSQCVNLRTRTQLPPAYTNSFTRGVYYPDKVFRSSVL
HSTQDLFLPFFSNVTWFHAIHVSGTNGTKREDNPVLPFNDGVYFASTE
KSNIIRGWIFGTTLDSKTQSLLIVNNATNVVIKVCEFQFCNDPFLDVY
YHKNNKSWMESGVYSSANNCTFEYVSQPFLMDLEGKQGNFKNLREFVE
KNIDGYFKIYSKHTPINLVRDLPQGFSALEPLVDLPIGINITRFQTLL
ALHRSYLTPGDSSSGWTAGAAAYYVGYLQPRTFLLKYNENGTITDAVD
CALDPLSETKCTLKSFTVEKGIYQTSNERVQPTESIVRFPNITNLCPF
GEVENATRFASVYAWNRKRISNCVADYSVLYNSASFSTFKCYGVSPTK
LNDLCFTNVYADSFVIRGDEVRQIAPGQTGNIADYNYKLPDDFTGCVI
AWNSNNLDSKVGGNYNYRYRLFRKSNLKPFERDISTEIYQAGSKPCNG
VEGFNCYFPLQSYGFQPTNGVGYQPYRVVVLSFELLHAPATVCGPKKS
TNLVKNKCVNFNFNGLTGTGVLTESNKKFLPFQQFGRDIADTTDAVRD
PQTLEILDITPCSFGGVSVITPGTNTSNQVAVLYQGVNCTEVPVAIHA
DQLTPTWRVYSTGSNVFQTRAGCLIGAEHVNNSYECDIPIGAGICASY
QTQTNSRRRARSVASQSIIAYTMSLGAENSVAYSNNSIAIPTNFTISV
TTEILPVSMTKTSVDCTMYICGDSTECSNLLLQYGSFCTQLNRALTGI
AVEQDKNTQEVFAQVKQIYKTPPIKDFGGFNFSQILPDPSKPSKRSFI
EDLLFNKVTLADAGFIKQYGDCLGDIAARDLICAQKFNGLTVLPPLLT
DEMIAQYTSALLAGTITSGWTFGAGAALQIPFAMQMAYRFNGIGVTQN
VLYENQKLIANQFNSAIGKIQDSLSSTASALGKLQNVVNQNAQALNTL
VKQLSSNFGAISSVLNDILSRLDPPEAEVQIDRLITGRLQSLQTYVTQ
QLIRAAEIRASANLAATKMSECVLGQSKRVDECGKGYHLMSFPQSAPH
GVVFLHVTYVPAQEKNFTTAPAICHDGKAHFPREGVFVSNGTHWFVTQ
RNFYEPQIITTDNTFVSGNCDVVIGIVNNTVYDPLQPELDSFKEELDK
YFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSC
GSCCKFDEDDSEPVLKGVKLHYT
SEQ ID NO: 12 is the amino acid sequence
of a stabilized SARS-CoV-2 omicron variant
spike protein with a double proline
substitution.
MFVFLVLLPLVSSQCVNLTTRTQLPPAYTNSFTRGVYYPDKVFRSSVL
HSTQDLFLPFFSNVTWFHVISGTNGTKRFDNPVLPFNDGVYFASIEKS
NIIRGWIFGTTLDSKTQSLLIVNNATNVVIKVCEFQFCNDPFLDHKNN
KSWMESEFRVYSSANNCTFEYVSQPFLMDLEGKQGNFKNLREFVFKNI
DGYFKIYSKHTPIIVEPERDLPQGFSALEPLVDLPIGINITRFQTLLA
LHRSYLTPGDSSSGWTAGAAAYYVGYLQPRTFLLKYNENGTITDAVDC
ALDPLSETKCTLKSFTVEKGIYQTSNERVQPTESIVRFPNITNLCPFD
EVENATRFASVYAWNRKRISNCVADYSVLYNLAPFFTFKCYGVSPTKL
NDLCFTNVYADSFVIRGDEVRQIAPGQTGNIADYNYKLPDDFTGCVIA
WNSNKLDSKVSGNYNYLYRLFRKSNLKPFERDISTEIYQAGNKPCNGV
AGENCYFPLRSYSFRPTYGVGHQPYRVVVLSFELLHAPATVCGPKKST
NLVKNKCVNFNFNGLKGTGVLTESNKKELPFQQFGRDIADTTDAVRDP
QTLEILDITPCSFGGVSVITPGTNTSNQVAVLYQGVNCTEVPVAIHAD
QLTPTWRVYSTGSNVFQTRAGCLIGAEYVNNSYECDIPIGAGICASYQ
TQTKSHRRARSVASQSIIAYTMSLGAENSVAYSNNSIAIPTNFTISVT
TEILPVSMTKTSVDCTMYICGDSTECSNLLLQYGSFCTQLKRALTGIA
VEQDKNTQEVFAQVKQIYKTPPIKYFGGENESQILPDPSKPSKRSFIE
DLLENKVTLADAGFIKQYGDCLGDIAARDLICAQKFKGLTVLPPLLTD
EMIAQYTSALLAGTITSGWTFGAGAALQIPFAMQMAYRENGIGVTQNV
LYENQKLIANQFNSAIGKIQDSLSSTASALGKLQDVVNHNAQALNTLV
KQLSSKFGAISSVLNDIFSRLDPPEAEVQIDRLITGRLQSLQTYVTQQ
LIRAAEIRASANLAATKMSECVLGQSKRVDFCGKGYHLMSFPQSAPHG
VVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKY
FKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCG
SCCKFDEDDSEPVLKGVKLHYT
SEQ ID NO: 13 is a codon-optimized nucleic
acid sequence encoding a stabilized SARS-
CoV-2 beta variant spike protein with a
double proline substitution.
ATGTTCGTGTTTCTGGTGCTGCTGCCTCTGGTGAGCTCCCAGTGCGTG
AACTTCACCACAAGAACCCAGCTGCCCCCTGCCTACACCAATTCCTTC
ACAAGGGGCGTGTACTATCCCGACAAGGTGTTTCGCTCTAGCGTGCTG
CACTCCACACAGGATCTGTTTCTGCCTTTCTTTTCTAACGTGACCTGG
TTCCACGCCATCCACGTGAGCGGCACCAATGGCACAAAGCGGTTCGCC
AATCCAGTGCTGCCCTTTAACGACGGCGTGTACTTCGCCTCCACCGAG
AAGTCTAACATCATCAGAGGCTGGATCTTTGGCACCACACTGGATAGC
AAGACACAGTCCCTGCTGATCGTGAACAATGCCACCAACGTGGTCATC
AAGGTGTGCGAGTTCCAGTTTTGTAATGACCCATTCCTGGGCGTGTAC
TATCACAAGAACAATAAGTCTTGGATGGAGAGCGAGTTTAGGGTGTAC
TCCTCTGCCAACAATTGCACATTTGAGTACGTGAGCCAGCCCTTCCTG
ATGGACCTGGAGGGCAAGCAGGGCAATTTCAAGAACCTGCGCGAGTTC
GTGTTTAAGAATATCGATGGCTACTTCAAGATCTACTCCAAGCACACC
CCAATCAACCTGGTGAGGGGACTGCCACAGGGCTTCTCTGCCCTGGAG
CCACTGGTGGACCTGCCCATCGGCATCAACATCACCCGCTTTCAGACA
CTGCACATCAGCTACCTGACACCAGGCGATAGCTCCTCTGGATGGACC
GCAGGAGCAGCAGCCTACTATGTGGGCTACCTGCAGCCCAGGACCTTC
CTGCTGAAGTATAACGAGAATGGCACCATCACAGACGCAGTGGATTGC
GCCCTGGACCCCCTGTCTGAGACCAAGTGTACACTGAAGAGCTTTACC
GTGGAGAAGGGCATCTACCAGACAAGCAATTTCCGGGTGCAGCCTACC
GAGTCCATCGTGAGATTTCCCAATATCACAAACCTGTGCCCTTTTGGC
GAGGTGTTCAACGCCACCCGCTTCGCCAGCGTGTATGCCTGGAATAGG
AAGCGCATCTCCAACTGCGTGGCCGACTATTCTGTGCTGTACAACAGC
GCCTCCTTCTCTACCTTTAAGTGCTACGGCGTGAGCCCCACAAAGCTG
AATGACCTGTGCTTTACCAACGTGTATGCCGATTCCTTCGTGATCAGG
GGCGACGAGGTGCGCCAGATCGCACCAGGCCAGACAGGCAATATCGCC
GACTACAACTATAAGCTGCCTGACGATTTCACCGGCTGCGTGATCGCC
TGGAACAGCAACAATCTGGATAGCAAAGTGGGCGGCAACTACAATTAT
CTGTACCGGCTGTTTAGAAAGTCTAACCTGAAGCCATTCGAGAGGGAC
ATCTCCACAGAGATCTACCAGGCCGGCTCTACCCCCTGCAATGGCGTG
AAGGGCTTTAACTGTTATTTCCCTCTGCAGAGCTACGGCTTCCAGCCA
ACCTACGGCGTGGGCTATCAGCCCTACCGCGTGGTGGTGCTGTCTTTT
GAGCTGCTGCACGCACCTGCAACAGTGTGCGGCCCAAAGAAGAGCACC
AATCTGGTGAAGAACAAGTGCGTGAACTTCAACTTCAACGGACTGACC
GGCACAGGCGTGCTGACCGAGTCCAACAAGAAGTTCCTGCCTTTTCAG
CAGTTCGGCCGGGACATCGCCGATACCACAGACGCCGTGAGAGACCCT
CAGACCCTGGAGATCCTGGATATCACACCATGCTCCTTCGGCGGCGTG
TCTGTGATCACACCAGGCACCAATACAAGCAACCAGGTGGCCGTGCTG
TACCAGGGCGTGAATTGTACCGAGGTGCCCGTGGCAATCCACGCAGAC
CAGCTGACCCCTACATGGAGGGTGTATTCTACCGGCAGCAACGTGTTC
CAGACACGCGCCGGATGCCTGATCGGAGCAGAGCACGTGAACAATAGC
TACGAGTGCGATATCCCTATCGGCGCCGGCATCTGTGCCTCCTATCAG
ACCCAGACAAACTCCCCACGGAGAGCCCGGTCTGTGGCAAGCCAGTCC
ATCATCGCCTACACCATGAGCCTGGGCGTGGAGAACAGCGTGGCCTAT
TCCAACAATTCTATCGCCATCCCTACCAACTTCACAATCTCCGTGACC
ACAGAGATCCTGCCAGTGAGCATGACCAAGACATCCGTGGACTGCACA
ATGTACATCTGTGGCGATTCCACCGAGTGCTCTAACCTGCTGCTGCAG
TATGGCTCTTTTTGTACCCAGCTGAATAGAGCCCTGACAGGCATCGCC
GTGGAGCAGGACAAGAACACACAGGAGGTGTTCGCCCAGGTGAAGCAG
ATCTACAAGACCCCACCCATCAAGGACTTTGGCGGCTTCAACTTCAGC
CAGATCCTGCCCGATCCTAGCAAGCCATCCAAGCGGTCTTTTATCGAG
GACCTGCTGTTCAACAAGGTGACCCTGGCCGATGCCGGCTTCATCAAG
CAGTACGGCGATTGCCTGGGCGACATCGCAGCCAGAGACCTGATCTGT
GCCCAGAAGTTTAATGGCCTGACCGTGCTGCCTCCACTGCTGACAGAT
GAGATGATCGCCCAGTATACATCTGCCCTGCTGGCAGGAACCATCACA
AGCGGATGGACCTTCGGCGCAGGAGCCGCCCTGCAGATCCCCTTTGCC
ATGCAGATGGCCTACAGGTTCAACGGCATCGGCGTGACCCAGAATGTG
CTGTATGAGAACCAGAAGCTGATCGCCAATCAGTTTAACTCCGCCATC
GGCAAGATCCAGGACTCTCTGAGCTCCACAGCAAGCGCCCTGGGCAAG
CTGCAGGATGTGGTGAATCAGAACGCCCAGGCCCTGAATACCCTGGTG
AAGCAGCTGTCTAGCAACTTCGGCGCCATCTCCTCTGTGCTGAATGAT
ATCCTGAGCCGGCTGGACCCTCCTGAGGCAGAGGTGCAGATCGACCGG
CTGATCACAGGCAGACTGCAGTCCCTGCAGACCTACGTGACACAGCAG
CTGATCAGGGCAGCAGAGATCAGGGCATCTGCCAATCTGGCCGCCACC
AAGATGAGCGAGTGCGTGCTGGGCCAGTCCAAGAGAGTGGACTTTTGT
GGCAAGGGCTACCACCTGATGAGCTTCCCACAGTCCGCCCCTCACGGC
GTGGTGTTTCTGCACGTGACCTATGTGCCAGCCCAGGAGAAGAACTTC
ACCACAGCACCAGCCATCTGCCACGATGGCAAGGCACACTTTCCTCGG
GAGGGCGTGTTCGTGAGCAACGGCACCCACTGGTTTGTGACACAGAGA
AATTTCTACGAGCCACAGATCATCACCACAGACAATACCTTCGTGAGC
GGCAACTGTGACGTGGTCATCGGAATCGTGAACAATACCGTGTACGAT
CCTCTGCAGCCAGAGCTGGACTCTTTTAAGGAGGAGCTGGATAAGTAT
TTCAAGAATCACACCAGCCCCGACGTGGATCTGGGCGACATCTCTGGC
ATCAATGCCAGCGTGGTGAACATCCAGAAGGAGATCGACCGCCTGAAC
GAGGTGGCCAAGAATCTGAACGAGTCCCTGATCGATCTGCAGGAGCTG
GGCAAGTATGAGCAGTACATCAAGTGGCCCTGGTACATCTGGCTGGGC
TTCATCGCCGGCCTGATCGCCATCGTGATGGTGACCATCATGCTGTGC
TGTATGACAAGCTGCTGTTCCTGCCTGAAGGGCTGCTGTTCTTGTGGC
AGCTGCTGTAAGTTTGATGAGGACGATAGCGAGCCTGTGCTGAAGGGC
GTGAAGCTGCACTATACCTGA
SEQ ID NO: 14 is a codon-optimized nucleic
acid sequence encoding a stabilized, double
proline-substituted, chimeric SARS-CoV-2
spike protein comprising the RBD of the beta
variant and remaining sequence from the
Wuhan strain.
ATGTTCGTGTTTCTGGTGCTGCTGCCTCTGGTGAGCTCCCAGTGCGTG
AACCTGACCACAAGGACCCAGCTGCCCCCTGCCTACACCAATTCCTTC
ACACGGGGCGTGTACTATCCCGACAAGGTGTTTAGATCTAGCGTGCTG
CACTCCACACAGGATCTGTTTCTGCCTTTCTTTTCTAACGTGACCTGG
TTCCACGCCATCCACGTGAGCGGCACCAATGGCACAAAGCGGTTCGAC
AATCCAGTGCTGCCCTTTAACGATGGCGTGTACTTCGCCTCCACCGAG
AAGTCTAACATCATCAGAGGCTGGATCTTTGGCACCACACTGGACAGC
AAGACACAGTCCCTGCTGATCGTGAACAATGCCACCAACGTGGTCATC
AAGGTGTGCGAGTTCCAGTTTTGTAATGATCCATTCCTGGGCGTGTAC
TATCACAAGAACAATAAGTCTTGGATGGAGAGCGAGTTTCGCGTGTAC
TCCTCTGCCAACAATTGCACATTTGAGTACGTGAGCCAGCCCTTCCTG
ATGGACCTGGAGGGCAAGCAGGGCAATTTCAAGAACCTGAGGGAGTTC
GTGTTTAAGAATATCGATGGCTACTTCAAGATCTACTCCAAGCACACC
CCAATCAACCTGGTGCGCGACCTGCCACAGGGCTTCTCTGCCCTGGAG
CCACTGGTGGATCTGCCCATCGGCATCAACATCACCCGGTTTCAGACA
CTGCTGGCCCTGCACAGAAGCTACCTGACACCAGGCGACAGCTCCTCT
GGATGGACCGCAGGAGCAGCAGCCTACTATGTGGGCTACCTGCAGCCC
AGGACCTTCCTGCTGAAGTATAACGAGAATGGCACCATCACAGACGCA
GTGGATTGCGCCCTGGACCCCCTGTCTGAGACCAAGTGTACACTGAAG
AGCTTTACCGTGGAGAAGGGCATCTACCAGACAAGCAATTTCAGGGTG
CAGCCTACCGAGTCCATCGTGCGCTTTCCCAATATCACAAACCTGTGC
CCTTTTGGCGAGGTGTTCAACGCCACCCGCTTCGCCAGCGTGTATGCC
TGGAATAGGAAGCGCATCTCCAACTGCGTGGCCGACTATTCTGTGCTG
TACAACAGCGCCTCCTTCTCTACCTTTAAGTGCTACGGCGTGAGCCCC
ACAAAGCTGAATGACCTGTGCTTTACCAACGTGTATGCCGATTCCTTC
GTGATCAGGGGCGACGAGGTGCGCCAGATCGCACCAGGCCAGACAGGC
AATATCGCCGACTACAACTATAAGCTGCCTGACGATTTCACCGGCTGC
GTGATCGCCTGGAACAGCAACAATCTGGATAGCAAAGTGGGCGGCAAC
TACAATTATCTGTACCGGCTGTTTAGAAAGTCTAACCTGAAGCCATTC
GAGAGGGACATCTCCACAGAGATCTACCAGGCCGGCTCTACCCCCTGC
AATGGCGTGAAGGGCTTTAACTGTTATTTCCCTCTGCAGAGCTACGGC
TTCCAGCCAACCTACGGCGTGGGCTATCAGCCCTACCGCGTGGTGGTG
CTGTCTTTTGAGCTGCTGCACGCACCTGCAACAGTGTGCGGCCCAAAG
AAGAGCACCAATCTGGTGAAGAACAAGTGCGTGAACTTCAACTTCAAC
GGACTGACCGGCACAGGCGTGCTGACCGAGTCCAACAAGAAGTTCCTG
CCTTTTCAGCAGTTCGGCAGGGACATCGCAGATACCACAGACGCCGTG
CGCGACCCTCAGACCCTGGAGATCCTGGATATCACACCATGCTCCTTC
GGCGGCGTGTCTGTGATCACACCAGGCACCAATACAAGCAACCAGGTG
GCCGTGCTGTACCAGGGCGTGAATTGTACCGAGGTGCCCGTGGCAATC
CACGCAGACCAGCTGACCCCTACATGGCGGGTGTATTCTACCGGCAGC
AACGTGTTCCAGACAAGAGCCGGATGCCTGATCGGAGCAGAGCACGTG
AACAATAGCTACGAGTGCGATATCCCTATCGGCGCCGGCATCTGTGCC
TCCTATCAGACCCAGACAAACTCCCCACGGAGAGCCCGGTCTGTGGCA
AGCCAGTCCATCATCGCCTACACCATGAGCCTGGGCGCCGAGAACAGC
GTGGCCTATTCCAACAATTCTATCGCCATCCCTACCAACTTCACAATC
TCCGTGACCACAGAGATCCTGCCAGTGAGCATGACCAAGACATCCGTG
GACTGCACAATGTACATCTGTGGCGATTCCACCGAGTGCTCTAACCTG
CTGCTGCAGTATGGCTCTTTTTGTACCCAGCTGAATAGAGCCCTGACA
GGCATCGCCGTGGAGCAGGACAAGAACACACAGGAGGTGTTCGCCCAG
GTGAAGCAGATCTACAAGACCCCACCCATCAAGGACTTTGGCGGCTTC
AACTTCAGCCAGATCCTGCCCGATCCTAGCAAGCCATCCAAGCGGTCT
TTTATCGAGGACCTGCTGTTCAACAAGGTGACCCTGGCCGATGCCGGC
TTCATCAAGCAGTACGGCGATTGCCTGGGCGACATCGCAGCCAGAGAC
CTGATCTGTGCCCAGAAGTTTAATGGCCTGACCGTGCTGCCTCCACTG
CTGACAGATGAGATGATCGCCCAGTATACATCTGCCCTGCTGGCAGGA
ACCATCACAAGCGGATGGACCTTCGGCGCAGGAGCCGCCCTGCAGATC
CCCTTTGCCATGCAGATGGCCTACAGATTCAACGGCATCGGCGTGACC
CAGAATGTGCTGTATGAGAACCAGAAGCTGATCGCCAATCAGTTTAAC
TCCGCCATCGGCAAGATCCAGGACTCTCTGAGCTCCACAGCAAGCGCC
CTGGGCAAGCTGCAGGATGTGGTGAATCAGAACGCCCAGGCCCTGAAT
ACCCTGGTGAAGCAGCTGTCTAGCAACTTCGGCGCCATCTCCTCTGTG
CTGAATGATATCCTGAGCCGGCTGGACCCACCAGAGGCAGAGGTGCAG
ATCGACCGGCTGATCACAGGCAGACTGCAGTCCCTGCAGACCTACGTG
ACACAGCAGCTGATCAGGGCAGCAGAGATCAGGGCATCTGCCAATCTG
GCCGCCACCAAGATGAGCGAGTGCGTGCTGGGCCAGTCCAAGAGAGTG
GACTTTTGTGGCAAGGGCTACCACCTGATGAGCTTCCCACAGTCCGCC
CCTCACGGCGTGGTGTTTCTGCACGTGACCTATGTGCCAGCCCAGGAG
AAGAACTTCACCACAGCACCAGCCATCTGCCACGATGGCAAGGCACAC
TTTCCCCGGGAGGGCGTGTTCGTGAGCAACGGAACCCACTGGTTTGTG
ACACAGCGCAATTTCTACGAGCCACAGATCATCACCACAGACAATACA
TTCGTGTCCGGCAACTGTGACGTGGTCATCGGAATCGTGAACAATACC
GTGTACGATCCTCTGCAGCCAGAGCTGGACTCTTTTAAGGAGGAGCTG
GATAAGTATTTCAAGAATCACACCAGCCCCGACGTGGATCTGGGCGAC
ATCTCTGGCATCAATGCCAGCGTGGTGAACATCCAGAAGGAGATCGAC
AGGCTGAACGAGGTGGCCAAGAATCTGAACGAGTCCCTGATCGATCTG
CAGGAGCTGGGCAAGTATGAGCAGTACATCAAGTGGCCCTGGTACATC
TGGCTGGGCTTCATCGCCGGCCTGATCGCCATCGTGATGGTGACCATC
ATGCTGTGCTGTATGACAAGCTGCTGTTCCTGCCTGAAGGGCTGCTGT
TCTTGTGGCAGCTGCTGTAAGTTTGATGAGGACGATAGCGAGCCTGTG
CTGAAGGGCGTGAAGCTGCACTATACCTGA
SEQ ID NO: 15 is a codon-optimized nucleic
acid sequence encoding a stabilized SARS-
CoV-2 delta variant spike protein with a
double proline substitution.
ATGTTCGTGTTTCTGGTGCTGCTGCCTCTGGTGAGCTCCCAGTGCGTG
AACCTGACCACAACCACACAGCTGCCCCCTGCCTATACCAATTCCTTC
ACACGCGGCGTGTACTATCCTGACAAGGTGTTTCGGTCTAGCGTGCTG
CACTCCACACAGGATCTGTTTCTGCCATTCTTTTCTAACGTGACCTGG
TTCCACGCCATCCACGTGAGCGGCACCAATGGCACAAAGCGGTTCGAC
AATCCAGTGCTGCCCTTTAACGATGGCGTGTACTTCGCCTCCACCGAG
AAGTCTAACATCATCCGGGGCTGGATCTTTGGCACCACACTGGACAGC
AAGACACAGTCCCTGCTGATCGTGAACAATGCCACCAACGTGGTCATC
AAGGTGTGCGAGTTCCAGTTTTGTAATGATCCCTTCCTGGACGTGTAC
TATCACAAGAACAATAAGTCTTGGATGAAGAGCGAGTTTAGAGTGTAT
TCCTCTGCCAACAATTGCACATTTGAGTACGTGTCCCAGCCTTTCCTG
ATGGACCTGGAGGGCAAGCAGGGCAATTTCAAGAACCTGAGAGAGTTC
GTGTTTAAGAATATCGATGGCTACTTCAAGATCTACTCCAAGCACACC
CCAATCAACCTGGTGAGGGACCTGCCACACGGCTTCTCTGCCCTGGAG
CCACTGGTGGATCTGCCCATCGGCATCAACATCACCAGATTTCAGACA
CTGCTGGCCCTGCACAGGAGCTACCTGACACCCGGCGACAGCTCCTCT
GGATGGACCGCCGGCGCTGCCGCCTACTATGTGGGCTATCTGCAGCCT
CGCACCTTCCTGCTGAAGTACAACGAGAATGGCACCATCACAGACGCA
GTGGATTGCGCCCTGGACCCCCTGTCTGAGACCAAGTGTACACTGAAG
AGCTTTACCGTGGAGAAGGGCATCTATCAGACAAGCAATTTCCGCGTG
CAGCCAACCGAGTCCATCGTGCGGTTTCCCAATATCACAAACCTGTGC
CCTTTTGGCGAGGTGTTCAACGCAACCAGGTTCGCAAGCGTGTACGCA
TGGAATCGCAAGCGGATCTCCAACTGCGTGGCCGACTATTCTGTGCTG
TACAACAGCGCCTCCTTCTCTACCTTTAAGTGCTATGGCGTGAGCCCA
ACAAAGCTGAATGACCTGTGCTTTACCAACGTGTACGCCGATTCCTTC
GTGATCCGGGGCGACGAGGTGCGGCAGATCGCACCAGGACAGACAGGC
AAGATCGCAGACTACAATTATAAGCTGCCTGACGATTTCACCGGCTGC
GTGATCGCCTGGAACTCTAACAATCTGGATAGCAAAGTGGGCGGCAAC
TACAATTATAGATACAGGCTGTTTAGAAAGTCTAATCTGAAGCCATTC
GAGAGGGACATCTCCACAGAGATCTACCAGGCCGGCTCTACCCCCTGC
AATGGCGTGCAGGGCTTTAACTGTTATTTCCCTCTGCAGAGCTACGGC
TTCCAGCCAACCAACGGCGTGGGCTATCAGCCCTACCGGGTGGTGGTG
CTGTCTTTTGAGCTGCTGCACGCACCTGCAACAGTGTGCGGACCAAAG
AAGAGCACCAATCTGGTGAAGAACAAGTGCGTGAACTTCAACTTCAAC
GGACTGACCGGAACAGGCGTGCTGACCGAGTCCAACAAGAAGTTCCTG
CCATTTCAGCAGTTCGGCAGAGACATCGCCGATACCACAGACGCCGTG
AGGGACCCTCAGACCCTGGAGATCCTGGATATCACACCATGCTCCTTC
GGCGGCGTGTCTGTGATCACACCCGGCACCAATACAAGCAACCAGGTG
GCCGTGCTGTATCAGGGCGTGAATTGTACCGAGGTGCCAGTGGCAATC
CACGCAGACCAGCTGACCCCTACATGGCGCGTGTACTCTACCGGCAGC
AACGTGTTCCAGACAAGGGCAGGATGCCTGATCGGAGCAGAGCACGTG
AACAATAGCTATGAGTGCGATATCCCCATCGGCGCCGGCATCTGTGCC
TCCTACCAGACCCAGACAAACTCCCGGAGAAGGGCCAGATCTGTGGCC
AGCCAGTCCATCATCGCCTATACCATGAGCCTGGGCGCCGAGAACAGC
GTGGCCTACTCCAACAATTCTATCGCCATCCCTACCAACTTCACAATC
TCCGTGACCACAGAGATCCTGCCAGTGAGCATGACCAAGACATCCGTG
GACTGCACAATGTATATCTGTGGCGATTCCACCGAGTGCTCTAACCTG
CTGCTGCAGTACGGCTCTTTTTGTACCCAGCTGAATAGGGCCCTGACA
GGAATCGCAGTGGAGCAGGACAAGAACACACAGGAGGTGTTCGCCCAG
GTGAAGCAGATCTACAAGACCCCACCCATCAAGGACTTTGGCGGCTTC
AACTTCAGCCAGATCCTGCCCGATCCTAGCAAGCCCTCCAAGCGGAGC
TTCATCGAGGACCTGCTGTTCAACAAGGTGACCCTGGCCGATGCCGGC
TTCATCAAGCAGTATGGCGATTGCCTGGGCGACATCGCAGCAAGGGAC
CTGATCTGTGCCCAGAAGTTTAATGGCCTGACCGTGCTGCCTCCACTG
CTGACAGATGAGATGATCGCCCAGTACACATCTGCCCTGCTGGCAGGA
ACCATCACAAGCGGATGGACCTTCGGCGCAGGAGCCGCCCTGCAGATC
CCTTTTGCCATGCAGATGGCCTATCGCTTCAACGGCATCGGCGTGACC
CAGAATGTGCTGTACGAGAACCAGAAGCTGATCGCCAATCAGTTTAAC
TCCGCCATCGGCAAGATCCAGGACTCTCTGAGCTCCACAGCAAGCGCC
CTGGGCAAGCTGCAGGATGTGGTGAATCAGAACGCCCAGGCCCTGAAT
ACCCTGGTGAAGCAGCTGTCTAGCAACTTCGGCGCCATCTCCTCTGTG
CTGAATGATATCCTGAGCAGACTGGACCCCCCCGAGGCCGAGGTGCAG
ATCGACAGACTGATCACAGGCAGGCTGCAGTCCCTGCAGACCTACGTG
ACACAGCAGCTGATCAGGGCCGCCGAGATCAGGGCCTCTGCCAATCTG
GCCGCCACCAAGATGAGCGAGTGCGTGCTGGGCCAGTCCAAGAGGGTG
GATTTTTGTGGCAAGGGCTATCACCTGATGAGCTTCCCACAGTCCGCC
CCTCACGGAGTGGTGTTTCTGCACGTGACCTACGTGCCAGCCCAGGAG
AAGAACTTCACCACAGCACCAGCAATCTGCCACGACGGCAAGGCACAC
TTTCCAAGAGAGGGCGTGTTCGTGAGCAACGGCACCGATTGGTTTGTG
ACACAGAGGAATTTCTACGAGCCCCAGATCATCACCACAGACAATACA
TTCGTGTCCGGCAACTGTGACGTGGTCATCGGCATCGTGAACAATACC
GTGTATGATCCTCTGCAGCCAGAGCTGGACTCTTTTAAGGAGGAGCTG
GATAAGTACTTCAAGAATCACACCAGCCCCGACGTGGATCTGGGCGAC
ATCTCTGGCATCAATGCCAGCGTGGTGAACATCCAGAAGGAGATCGAC
CGGCTGAACGAGGTGGCCAAGAATCTGAACGAGTCCCTGATCGATCTG
CAGGAGCTGGGCAAGTATGAGCAGTACATCAAGTGGCCTTGGTATATC
TGGCTGGGCTTCATCGCCGGCCTGATCGCCATCGTGATGGTGACCATC
ATGCTGTGCTGTATGACAAGCTGCTGTTCCTGCCTGAAGGGCTGCTGT
TCTTGTGGCAGCTGCTGTAAGTTTGATGAGGACGATAGCGAGCCAGTG
CTGAAGGGCGTGAAGCTGCACTACACCTGA
SEQ ID NO: 16 is a codon-optimized nucleic
acid sequence encoding a stabilized SARS-
CoV-2 gamma variant spike protein with a
double proline substitution.
ATGTTCGTGTTTCTGGTGCTGCTGCCTCTGGTGAGCTCCCAGTGCGTG
AATTTCACCAACAGAACACAGCTGCCTTCTGCCTACACCAATAGCTTC
ACACGGGGCGTGTACTATCCAGACAAGGTGTTTAGATCTAGCGTGCTG
CACAGCACACAGGATCTGTTTCTGCCATTCTTTTCCAACGTGACCTGG
TTCCACGCCATCCACGTGTCCGGCACCAATGGCACAAAGCGGTTCGAC
AATCCCGTGCTGCCTTTTAACGATGGCGTGTACTTCGCCTCCACCGAG
AAGTCTAACATCATCAGAGGCTGGATCTTTGGCACCACACTGGACAGC
AAGACACAGTCCCTGCTGATCGTGAACAATGCCACCAACGTGGTCATC
AAGGTGTGCGAGTTCCAGTTTTGTAATTATCCCTTCCTGGGCGTGTAC
TATCACAAGAACAATAAGTCTTGGATGGAGAGCGAGTTTAGGGTGTAC
TCCTCTGCCAACAATTGCACATTTGAGTATGTGAGCCAGCCTTTCCTG
ATGGACCTGGAGGGCAAGCAGGGCAATTTCAAGAACCTGAGCGAGTTC
GTGTTTAAGAATATCGATGGCTACTTCAAGATCTACTCCAAGCACACC
CCCATCAACCTGGTGCGCGACCTGCCTCAGGGCTTCTCTGCCCTGGAG
CCCCTGGTGGATCTGCCTATCGGCATCAACATCACCCGGTTTCAGACA
CTGCTGGCCCTGCACAGAAGCTACCTGACACCCGGCGACAGCTCCTCT
GGATGGACCGCCGGCGCTGCCGCCTACTATGTGGGCTACCTGCAGCCT
AGGACCTTCCTGCTGAAGTATAACGAGAATGGCACCATCACAGACGCA
GTGGATTGCGCCCTGGACCCCCTGTCCGAGACCAAGTGTACACTGAAG
TCTTTTACCGTGGAGAAGGGCATCTACCAGACATCTAATTTCAGGGTG
CAGCCAACCGAGAGCATCGTGCGCTTTCCTAATATCACAAACCTGTGC
CCATTTGGCGAGGTGTTCAACGCCACCCGCTTCGCCAGCGTGTATGCC
TGGAATAGGAAGCGCATCAGCAACTGCGTGGCCGACTATTCCGTGCTG
TACAACAGCGCCTCCTTCTCTACCTTTAAGTGTTACGGCGTGTCTCCT
ACAAAGCTGAATGACCTGTGCTTTACCAACGTGTATGCCGATAGCTTC
GTGATCAGGGGCGACGAGGTGCGCCAGATCGCACCAGGACAGACCGGA
ACAATCGCAGACTACAATTATAAGCTGCCTGACGATTTCACCGGCTGC
GTGATCGCCTGGAACTCCAACAATCTGGATTCTAAAGTGGGCGGCAAC
TACAATTATCTGTACCGGCTGTTTAGAAAGTCCAACCTGAAGCCATTC
GAGCGGGACATCAGCACAGAGATCTACCAGGCAGGCTCCACCCCATGC
AATGGAGTGAAGGGCTTTAACTGTTATTTCCCACTGCAGAGCTACGGC
TTCCAGCCCACATATGGCGTGGGCTATCAGCCTTACAGAGTGGTGGTG
CTGTCCTTTGAGCTGCTGCACGCACCAGCAACAGTGTGCGGACCCAAG
AAGTCTACCAATCTGGTGAAGAACAAGTGCGTGAACTTCAACTTCAAC
GGACTGACCGGAACAGGCGTGCTGACCGAGTCCAACAAGAAGTTCCTG
CCATTTCAGCAGTTCGGCAGGGACATCGCAGATACCACAGACGCCGTG
CGCGACCCACAGACCCTGGAGATCCTGGATATCACACCCTGCAGCTTC
GGCGGCGTGTCCGTGATCACACCAGGAACCAATACAAGCAACCAGGTG
GCCGTGCTGTACCAGGGCGTGAATTGTACCGAGGTGCCTGTGGCAATC
CACGCAGACCAGCTGACCCCAACATGGCGGGTGTATTCTACCGGCAGC
AACGTGTTCCAGACAAGAGCCGGCTGCCTGATCGGCGCCGAGTATGTG
AACAATTCTTACGAGTGCGATATCCCTATCGGCGCCGGCATCTGTGCC
AGCTACCAGACCCAGACAAACAGCCCACGGAGAGCACGGTCCGTGGCA
AGCCAGTCCATCATCGCCTACACCATGTCTCTGGGCGCCGAGAATAGC
GTGGCCTATTCCAACAATTCTATCGCCATCCCAACCAACTTCACAATC
TCCGTGACCACAGAGATCCTGCCCGTGTCTATGACCAAGACAAGCGTG
GACTGCACAATGTACATCTGTGGCGATTCCACCGAGTGCTCTAACCTG
CTGCTGCAGTATGGCAGCTTTTGTACCCAGCTGAATAGAGCCCTGACA
GGCATCGCCGTGGAGCAGGACAAGAACACACAGGAGGTGTTCGCCCAG
GTGAAGCAGATCTACAAGACCCCCCCTATCAAGGACTTTGGCGGCTTC
AACTTCAGCCAGATCCTGCCTGATCCAAGCAAGCCATCCAAGAGGTCT
TTTATCGAGGACCTGCTGTTCAACAAGGTGACCCTGGCCGATGCCGGC
TTCATCAAGCAGTACGGCGATTGCCTGGGCGACATCGCAGCAAGGGAC
CTGATCTGTGCCCAGAAGTTTAATGGCCTGACCGTGCTGCCACCCCTG
CTGACAGATGAGATGATCGCCCAGTATACATCCGCCCTGCTGGCCGGC
ACCATCACATCTGGATGGACCTTCGGCGCAGGAGCCGCCCTGCAGATC
CCCTTTGCCATGCAGATGGCCTACAGGTTCAACGGCATCGGCGTGACC
CAGAATGTGCTGTATGAGAACCAGAAGCTGATCGCCAATCAGTTTAAC
TCCGCCATCGGCAAGATCCAGGACTCCCTGAGCTCCACAGCCTCTGCC
CTGGGCAAGCTGCAGGATGTGGTGAATCAGAACGCCCAGGCCCTGAAT
ACCCTGGTGAAGCAGCTGTCTAGCAACTTCGGCGCCATCTCCTCTGTG
CTGAATGATATCCTGAGCCGGCTGGACCCCCCCGAGGCAGAGGTGCAG
ATCGACCGGCTGATCACCGGCAGACTGCAGAGCCTGCAGACCTACGTG
ACACAGCAGCTGATCAGGGCCGCCGAGATCAGGGCATCCGCCAATCTG
GCCGCCATCAAGATGTCTGAGTGCGTGCTGGGCCAGAGCAAGAGAGTG
GACTTTTGTGGCAAGGGCTACCACCTGATGAGCTTCCCTCAGTCCGCC
CCACACGGAGTGGTGTTTCTGCACGTGACCTATGTGCCCGCCCAGGAG
AAGAACTTCACCACAGCCCCTGCCATCTGCCACGATGGCAAGGCCCAC
TTTCCAAGGGAGGGCGTGTTCGTGTCCAACGGCACCCACTGGTTTGTG
ACACAGCGCAATTTCTACGAGCCCCAGATCATCACCACAGACAATACC
TTCGTGAGCGGCAACTGTGACGTGGTCATCGGCATCGTGAACAATACC
GTGTACGATCCACTGCAGCCCGAGCTGGACTCCTTTAAGGAGGAGCTG
GATAAGTATTTCAAGAATCACACCTCTCCCGACGTGGATCTGGGCGAC
ATCTCCGGCATCAATGCCTCTTTCGTGAACATCCAGAAGGAGATCGAC
CGCCTGAACGAGGTGGCCAAGAATCTGAACGAGTCCCTGATCGATCTG
CAGGAGCTGGGCAAGTATGAGCAGTACATCAAGTGGCCCTGGTACATC
TGGCTGGGCTTCATCGCCGGCCTGATCGCCATCGTGATGGTGACCATC
ATGCTGTGCTGTATGACAAGCTGCTGTTCCTGCCTGAAGGGCTGCTGT
TCTTGTGGCAGCTGCTGTAAGTTTGATGAGGACGATAGCGAGCCTGTG
CTGAAGGGCGTGAAGCTGCACTATACCTGA
SEQ ID NO: 17 is a codon-optimized nucleic
acid sequence encoding a stabilized SARS-
CoV-2 delta plus variant spike protein with
a double proline substitution.
ATGTTTGTGTTTCTGGTGCTGCTGCCACTGGTGAGTAGCCAGTGTGTG
AACCTGAGAACCCGAACACAGCTGCCTCCTGCCTATACCAACAGCTTC
ACCAGAGGCGTGTACTACCCTGACAAGGTGTTCCGATCTAGCGTGCTC
CATAGCACCCAGGACCTGTTCTTGCCTTTTTTCTCTAACGTGACATGG
TTCCACGCCATTCACGTGTCTGGCACCAACGGAACAAAAAGATTCGAC
AACCCTGTGCTGCCCTTCAACGACGGTGTCTATTTTGCCAGCACCGAG
AAGAGCAACATCATCAGAGGCTGGATCTTCGGAACCACCCTGGACAGC
AAGACCCAGAGCCTGCTGATCGTCAATAACGCAACAAATGTGGTGATC
AAGGTGTGCGAGTTCCAATTTTGCAACGATCCTTTCCTGGATGTGTAC
TACCACAAGAACAACAAAAGCTGGATGGAAAGTGGAGTTTATAGCAGC
GCCAACAACTGCACCTTCGAGTACGTGAGCCAACCTTTCCTGATGGAC
CTCGAAGGGAAACAGGGCAACTTCAAGAACCTTAGAGAGTTCGTCTTT
AAGAACATCGACGGCTACTTTAAAATCTACTCCAAGCACACCCCCATC
AACCTGGTGCGGGACCTGCCTCAGGGCTTTAGCGCGCTGGAACCCTTG
GTTGACCTGCCCATCGGCATCAACATCACTAGATTCCAGACCCTTCTG
GCCCTCCACCGGTCTTACCTGACACCTGGCGACAGTAGTTCTGGCTGG
ACAGCCGGCGCCGCTGCCTACTACGTGGGCTATCTGCAGCCTAGAACC
TTCCTGCTGAAGTACAACGAGAACGGCACCATCACCGACGCTGTGGAT
TGCGCCCTGGACCCTCTGTCCGAAACCAAGTGCACACTGAAGTCCTTC
ACCGTGGAAAAGGGCATCTACCAGACCTCTAACTTCCGGGTGCAGCCT
ACTGAAAGCATCGTGCGGTTCCCAAACATTACAAACCTGTGCCCTTTC
GGAGAAGTTTTCAACGCCACTCGCTTCGCCTCTGTCTATGCCTGGAAC
AGAAAGCGGATCAGCAATTGTGTGGCCGATTACAGCGTGCTGTACAAC
AGCGCCAGCTTTTCTACATTCAAGTGCTACGGCGTGTCTCCCACCAAG
CTGAATGATCTGTGCTTCACCAACGTGTACGCCGACTCGTTTGTGATC
CGGGGAGACGAAGTGCGCCAGATCGCCCCTGGGCAGACAGGAAACATC
GCCGATTACAATTACAAACTGCCTGACGATTTTACAGGATGTGTGATA
GCTTGGAACTCCAACAACCTCGACAGCAAAGTGGGCGGCAACTACAAT
TACCGGTACAGACTGTTTAGAAAGAGCAACCTAAAACCCTTCGAGAGA
GATATCTCTACCGAGATCTACCAGGCCGGCAGCAAGCCTTGTAATGGC
GTTGAGGGCTTCAACTGTTACTTCCCTCTGCAGAGCTACGGCTTCCAG
CCCACCAACGGCGTCGGGTACCAGCCTTACAGAGTTGTGGTTCTGAGC
TTCGAGCTGCTCCACGCTCCTGCCACCGTGTGTGGTCCTAAGAAAAGC
ACCAACCTGGTGAAGAACAAGTGCGTGAATTTCAATTTCAACGGCCTG
ACAGGCACAGGCGTGCTGACCGAGAGCAACAAAAAGTTCCTGCCCTTC
CAGCAGTTCGGCAGAGATATTGCCGATACCACAGACGCCGTGCGGGAC
CCTCAAACCCTGGAAATCTTGGACATCACACCTTGCAGCTTCGGCGGA
GTGTCTGTGATCACTCCCGGGACCAACACCAGCAACCAGGTTGCCGTG
CTGTACCAGGGCGTCAACTGCACCGAAGTGCCAGTGGCTATACACGCC
GACCAGCTGACCCCTACATGGCGGGTGTACAGCACCGGCAGCAACGTG
TTCCAGACCAGAGCCGGCTGCCTGATCGGCGCAGAGCACGTGAACAAC
TCTTATGAATGCGACATCCCCATCGGAGCCGGCATTTGCGCCAGCTAC
CAGACACAGACCAATAGCAGAAGACGGGCTAGAAGCGTGGCCTCGCAG
AGCATAATCGCATACACAATGAGCCTGGGAGCCGAGAACAGCGTGGCC
TACAGCAACAATAGTATCGCCATCCCCACAAATTTTACCATCAGCGTG
ACAACCGAAATCCTGCCAGTGAGCATGACAAAGACCAGCGTCGACTGC
ACAATGTACATATGTGGCGATAGCACGGAGTGCAGCAATCTGCTGCTC
CAATACGGCAGCTTCTGCACCCAGCTGAATCGGGCACTGACCGGCATC
GCCGTGGAACAGGATAAAAATACCCAGGAGGTGTTTGCCCAGGTGAAG
CAGATATATAAGACCCCTCCGATCAAGGACTTCGGAGGCTTCAATTTC
AGCCAGATCCTGCCCGATCCAAGCAAGCCTAGCAAGCGGTCCTTCATC
GAGGATCTGCTGTTCAATAAGGTGACCCTGGCCGACGCCGGATTCATC
AAACAGTACGGCGACTGCCTGGGCGACATCGCCGCCAGAGATCTGATC
TGTGCTCAAAAGTTCAACGGACTGACAGTCCTGCCACCTCTGTTGACA
GATGAAATGATCGCTCAGTACACCTCCGCCCTCCTGGCCGGGACGATC
ACCTCTGGATGGACCTTCGGCGCCGGCGCTGCACTGCAGATCCCTTTC
GCCATGCAGATGGCCTACAGATTCAACGGCATCGGAGTGACCCAAAAC
GTCCTGTACGAGAACCAGAAGCTGATCGCCAACCAGTTCAACTCTGCT
ATCGGCAAGATCCAGGACAGCCTCAGCAGCACCGCCAGCGCCCTGGGC
AAACTCCAGAACGTGGTGAACCAGAACGCACAGGCCCTGAATACCCTG
GTGAAGCAGCTGAGCAGCAACTTCGGCGCTATCAGCTCTGTGCTGAAC
GACATCCTGAGCAGACTGGACCCTCCCGAGGCCGAGGTGCAGATTGAC
AGGCTGATCACAGGCAGACTGCAGTCGCTGCAAACTTACGTGACCCAG
CAACTGATCCGGGCCGCCGAAATCAGGGCCAGCGCCAACCTGGCTGCT
ACAAAGATGTCCGAATGCGTGTTGGGCCAGTCCAAGAGAGTGGACTTC
TGCGGCAAGGGATACCACCTGATGAGCTTCCCTCAGTCCGCTCCCCAC
GGCGTCGTGTTCCTGCATGTGACATACGTGCCCGCCCAGGAGAAGAAT
TTCACCACCGCCCCTGCCATCTGCCACGACGGCAAGGCCCACTTCCCC
AGAGAGGGCGTGTTCGTGTCCAACGGCACCCACTGGTTCGTGACCCAG
CGGAACTTCTACGAGCCTCAGATCATCACCACCGATAACACATTCGTG
TCCGGCAACTGCGACGTGGTTATCGGCATCGTGAACAATACCGTGTAC
GACCCTCTGCAGCCAGAACTGGATTCTTTTAAGGAAGAGCTGGACAAA
TACTTTAAGAACCACACATCTCCTGATGTGGACCTGGGCGACATCAGC
GGCATCAACGCCTCCGTGGTCAACATCCAAAAGGAGATCGATAGACTG
AACGAGGTGGCCAAGAACCTCAACGAGTCTCTGATTGACCTGCAGGAG
CTGGGCAAGTACGAGCAGTACATCAAGTGGCCTTGGTACATCTGGCTG
GGCTTCATCGCCGGCCTGATCGCTATCGTCATGGTGACCATCATGCTG
TGCTGTATGACCTCCTGCTGCAGCTGTCTGAAAGGCTGCTGTTCTTGC
GGCAGCTGTTGCAAGTTTGACGAGGACGACTCCGAGCCCGTGCTGAAG
GGGGTGAAGCTGCACTACACGTGA
SEQ ID NO: 18 is a codon-optimized nucleic
acid sequence encoding a stabilized SARS-
CoV-2 omicron variant spike protein with a
double proline substitution.
ATGTTCGTGTTCCTGGTGCTGCTGCCCCTGGTGTCTAGCCAATGTGTG
AACCTGACAACAAGGACCCAGCTTCCCCCAGCTTACACCAATTCATTT
ACAAGAGGCGTGTATTACCCCGATAAGGTGTTCCGAAGCAGCGTGCTG
CACAGCACCCAGGATCTCTTCCTGCCTTTTTTCAGCAATGTGACTTGG
TTCCACGTGATCAGCGGAACCAACGGCACCAAGCGGTTTGACAATCCT
GTGCTGCCCTTCAACGACGGCGTGTACTTCGCCAGCATCGAGAAGAGC
AACATTATCCGGGGCTGGATCTTCGGCACCACCCTCGATAGCAAGACC
CAGAGCTTACTGATCGTAAACAACGCCACCAATGTCGTAATCAAGGTC
TGTGAATTTCAGTTCTGCAACGACCCCTTTCTGGACCACAAGAACAAC
AAGTCGTGGATGGAAAGCGAGTTCAGAGTGTACAGCTCCGCTAACAAT
TGTACATTCGAGTACGTGTCTCAGCCTTTCCTGATGGACCTGGAAGGC
AAGCAGGGAAACTTCAAGAATCTGAGGGAGTTCGTGTTCAAAAACATC
GACGGCTACTTCAAGATCTACAGCAAGCATACCCCCATCATCGTTGAA
CCTGAGAGAGACCTGCCACAGGGTTTCAGCGCTCTGGAGCCTCTGGTT
GACCTGCCCATCGGCATCAACATCACCCGGTTTCAGACACTGTTAGCC
CTGCATAGATCTTACCTGACCCCAGGCGATTCTTCCTCTGGCTGGACC
GCCGGAGCCGCAGCCTACTACGTGGGATATCTGCAGCCCAGAACCTTC
CTGCTGAAATACAACGAGAACGGAACCATCACCGATGCCGTGGACTGC
GCCCTGGACCCTCTGTCTGAAACCAAGTGCACCCTGAAGAGCTTCACC
GTGGAAAAGGGCATCTACCAGACCAGCAACTTTCGGGTGCAGCCCACC
GAGAGCATCGTGAGATTTCCAAACATCACCAACCTGTGTCCTTTCGAC
GAGGTGTTTAATGCCACAAGATTCGCCAGCGTGTACGCCTGGAATAGA
AAAAGAATCTCCAACTGCGTGGCTGATTACTCAGTGCTTTACAACCTG
GCCCCATTCTTCACCTTCAAGTGCTACGGCGTTAGCCCTACCAAGCTC
AATGATCTGTGCTTCACGAACGTGTACGCCGACAGCTTCGTGATCCGG
GGCGACGAAGTCAGACAGATCGCCCCTGGACAGACCGGTAATATCGCC
GACTACAATTACAAGCTGCCTGATGATTTCACAGGTTGCGTGATCGCC
TGGAACTCCAACAAGCTGGACAGCAAGGTGTCCGGCAACTACAACTAC
CTGTATAGACTTTTCAGAAAGTCCAACCTGAAGCCATTCGAGCGGGAC
ATCAGCACTGAGATCTACCAGGCCGGCAACAAACCCTGCAACGGAGTT
GCCGGATTCAACTGCTATTTCCCTCTGAGATCTTACTCCTTCAGACCT
ACATACGGCGTGGGACACCAGCCTTACAGAGTAGTGGTGCTCAGCTTC
GAGCTTCTGCACGCTCCTGCCACCGTGTGCGGCCCTAAGAAGAGCACG
AACCTGGTGAAGAACAAATGTGTTAATTTTAACTTCAACGGCCTGAAG
GGCACAGGAGTCCTGACCGAGAGCAATAAAAAATTCTTGCCCTTCCAG
CAGTTCGGAAGAGACATCGCCGACACCACAGATGCTGTGAGAGACCCT
CAGACCCTGGAAATCCTCGACATCACCCCTTGCAGCTTCGGCGGCGTC
AGCGTGATCACCCCGGGCACCAACACCTCTAACCAGGTGGCCGTGCTG
TACCAGGGCGTGAATTGCACCGAGGTTCCTGTGGCCATCCACGCGGAC
CAGCTGACACCAACATGGCGGGTGTACAGCACCGGCTCCAACGTGTTT
CAGACCAGAGCCGGCTGTCTGATCGGCGCCGAATATGTGAACAACAGC
TACGAATGCGACATCCCAATCGGCGCCGGCATTTGCGCCAGCTACCAG
ACACAGACCAAAAGTCACCGGAGAGCTCGGAGCGTGGCCTCTCAGAGC
ATTATCGCCTATACCATGAGCCTGGGGGCCGAGAACAGCGTGGCCTAT
TCCAACAACAGCATCGCCATCCCTACCAATTTCACCATCTCTGTGACC
ACCGAGATCCTGCCAGTGTCCATGACAAAGACAAGCGTGGACTGCACC
ATGTACATCTGCGGCGACTCTACCGAGTGCAGCAACCTGCTGCTGCAG
TACGGCAGCTTTTGCACACAGCTGAAACGGGCGCTGACAGGAATTGCC
GTTGAGCAGGACAAGAACACTCAGGAGGTGTTTGCCCAAGTGAAGCAG
ATATATAAGACCCCTCCTATCAAATACTTCGGCGGCTTTAACTTCAGC
CAGATCCTCCCTGATCCTTCTAAGCCTAGCAAGCGCAGCTTCATCGAG
GACCTGCTGTTCAACAAGGTAACCCTGGCTGACGCCGGCTTCATCAAG
CAGTACGGTGATTGCCTGGGCGACATCGCAGCCCGGGACCTGATCTGT
GCCCAAAAATTCAAGGGCCTGACTGTTCTGCCTCCTCTGCTGACAGAT
GAAATGATCGCCCAGTACACCTCCGCCCTGCTGGCTGGCACAATCACC
AGCGGCTGGACATTCGGCGCCGGCGCCGCGCTGCAGATCCCTTTCGCC
ATGCAGATGGCCTACAGATTCAACGGCATCGGAGTGACTCAGAACGTG
CTGTACGAAAACCAGAAACTGATTGCAAATCAGTTTAACAGCGCAATC
GGCAAGATCCAGGATAGCCTGTCCAGCACCGCCTCCGCTCTGGGCAAG
CTGCAAGACGTGGTGAACCACAATGCCCAGGCTCTGAACACCTTGGTG
AAGCAGCTGAGCAGCAAGTTCGGCGCCATTTCTTCCGTGCTGAACGAC
ATCTTCAGCAGACTCGATCCTCCCGAGGCCGAGGTGCAGATCGACAGA
CTGATCACGGGCAGACTGCAGTCTCTGCAGACATACGTGACACAGCAA
CTGATCAGAGCCGCTGAAATCAGGGCCTCTGCCAACCTGGCCGCCACC
AAGATGTCTGAGTGCGTGCTCGGCCAGTCTAAAAGAGTGGACTTCTGC
GGCAAAGGCTACCACCTGATGAGCTTCCCCCAGAGCGCCCCCCACGGC
GTGGTGTTCCTACACGTTACCTACGTGCCGGCTCAAGAAAAGAACTTT
ACCACCGCCCCTGCCATCTGCCACGACGGAAAGGCCCACTTCCCTCGG
GAGGGTGTGTTTGTCAGCAACGGCACACACTGGTTCGTGACACAGCGG
AACTTCTACGAGCCCCAAATCATCACAACAGATAACACCTTCGTCAGC
GGCAACTGTGACGTGGTGATCGGCATCGTGAACAACACCGTGTATGAC
CCTCTGCAGCCTGAGCTGGACAGCTTTAAGGAAGAGCTGGACAAGTAC
TTCAAGAATCACACAAGTCCTGACGTGGATCTGGGCGATATCAGTGGC
ATCAACGCCTCTGTGGTGAACATACAAAAGGAGATCGACAGACTGAAC
GAGGTGGCAAAGAACCTGAATGAAAGCCTGATCGACCTGCAAGAACTG
GGCAAGTACGAGCAGTACATCAAGTGGCCTTGGTACATTTGGCTGGGA
TTTATCGCAGGCCTCATCGCCATCGTGATGGTGACAATCATGCTGTGT
TGCATGACCAGCTGTTGCAGCTGCCTGAAAGGCTGTTGTAGCTGCGGC
AGCTGCTGCAAGTTCGATGAGGACGACAGCGAGCCTGTCCTGAAGGGG
GTGAAGCTGCACTACACATGA
SEQ ID NO: 19 is a codon-optimized nucleic
acid sequence encoding a stabilized SARS-
CoV-2 Wuhan strain spike protein with a
double proline substitution.
ATGTTCGTCTTCCTGGTCCTGCTGCCCCTGGTCTCATCTCAGTGCGTG
AATCTGACTACAAGAACTCAGCTGCCTCCCGCCTACACCAATTCCTTC
ACCCGGGGCGTGTACTATCCTGACAAGGTGTTTAGAAGCTCCGTGCTG
CACTCTACACAGGATCTGTTTCTGCCATTCTTTAGCAACGTGACCTGG
TTCCACGCCATCCACGTGAGCGGCACCAATGGCACAAAGCGGTTCGAC
AATCCCGTGCTGCCTTTTAACGATGGCGTGTACTTCGCCTCTACCGAG
AAGAGCAACATCATCAGAGGCTGGATCTTTGGCACCACACTGGACTCC
AAGACACAGTCTCTGCTGATCGTGAACAATGCCACCAACGTGGTCATC
AAGGTGTGCGAGTTCCAGTTTTGTAATGATCCCTTCCTGGGCGTGTAC
TATCACAAGAACAATAAGAGCTGGATGGAGTCCGAGTTTAGAGTGTAT
TCTAGCGCCAACAATTGCACATTTGAGTACGTGTCCCAGCCTTTCCTG
ATGGACCTGGAGGGCAAGCAGGGCAATTTCAAGAACCTGAGGGAGTTC
GTGTTTAAGAATATCGATGGCTACTTCAAGATCTACTCTAAGCACACC
CCCATCAACCTGGTGCGCGACCTGCCTCAGGGCTTCAGCGCCCTGGAG
CCACTGGTGGATCTGCCTATCGGCATCAACATCACCCGGTTTCAGACA
CTGCTGGCCCTGCACAGAAGCTACCTGACACCCGGCGACTCCTCTAGC
GGATGGACCGCAGGAGCAGCAGCCTACTATGTGGGCTATCTGCAGCCT
AGGACCTTCCTGCTGAAGTACAACGAGAATGGCACCATCACAGACGCA
GTGGATTGCGCCCTGGACCCCCTGAGCGAGACAAAGTGTACACTGAAG
TCCTTTACCGTGGAGAAGGGCATCTATCAGACATCCAATTTCAGGGTG
CAGCCAACCGAGTCTATCGTGCGCTTTCCTAATATCACAAACCTGTGC
CCATTTGGCGAGGTGTTCAACGCAACCAGGTTCGCAAGCGTGTACGCA
TGGAATAGGAAGCGCATCTCTAACTGCGTGGCCGACTATAGCGTGCTG
TACAACTCCGCCTCTTTCAGCACCTTTAAGTGCTATGGCGTGTCCCCC
ACAAAGCTGAATGACCTGTGCTTTACCAACGTGTACGCCGATTCTTTC
GTGATCAGGGGCGACGAGGTGCGCCAGATCGCACCTGGACAGACAGGC
AAGATCGCCGACTACAATTATAAGCTGCCAGACGATTTCACCGGCTGC
GTGATCGCCTGGAACAGCAACAATCTGGATTCCAAAGTGGGCGGCAAC
TACAATTATCTGTACCGGCTGTTTAGAAAGAGCAATCTGAAGCCCTTC
GAGAGGGACATCTCTACAGAGATCTACCAGGCCGGCAGCACCCCTTGC
AATGGCGTGGAGGGCTTTAACTGTTATTTCCCACTGCAGTCCTACGGC
TTCCAGCCCACAAACGGCGTGGGCTATCAGCCTTACCGCGTGGTGGTG
CTGAGCTTTGAGCTGCTGCACGCACCAGCAACAGTGTGCGGACCCAAG
AAGTCCACCAATCTGGTGAAGAACAAGTGCGTGAACTTCAACTTCAAC
GGCCTGACCGGAACAGGCGTGCTGACCGAGTCCAACAAGAAGTTCCTG
CCATTTCAGCAGTTCGGCAGGGACATCGCAGATACCACAGACGCCGTG
CGCGACCCACAGACCCTGGAGATCCTGGATATCACACCCTGCTCTTTC
GGCGGCGTGAGCGTGATCACACCAGGAACCAATACAAGCAACCAGGTG
GCCGTGCTGTATCAGGACGTGAATTGTACCGAGGTGCCTGTGGCCATC
CACGCCGATCAGCTGACCCCAACATGGCGGGTGTACAGCACCGGCTCC
AACGTGTTCCAGACAAGAGCAGGATGCCTGATCGGAGCAGAGCACGTG
AACAATTCCTATGAGTGCGACATCCCAATCGGCGCCGGCATCTGTGCC
TCTTACCAGACCCAGACAAACTCTCCAAGGAGAGCACGGAGCGTGGCA
TCCCAGTCTATCATCGCCTATACCATGTCCCTGGGCGCCGAGAATTCT
GTGGCCTACTCTAACAATAGCATCGCCATCCCAACCAACTTCACAATC
TCTGTGACCACAGAGATCCTGCCCGTGTCCATGACCAAGACATCTGTG
GACTGCACAATGTATATCTGTGGCGATTCTACCGAGTGCAGCAACCTG
CTGCTGCAGTACGGCAGCTTTTGTACCCAGCTGAATAGAGCCCTGACA
GGCATCGCCGTGGAGCAGGATAAGAACACACAGGAGGTGTTCGCCCAG
GTGAAGCAGATCTACAAGACCCCCCCTATCAAGGACTTTGGCGGCTTC
AATTTTTCCCAGATCCTGCCTGATCCATCCAAGCCTTCTAAGCGGAGC
TTTATCGAGGACCTGCTGTTCAACAAGGTGACCCTGGCCGATGCCGGC
TTCATCAAGCAGTATGGCGATTGCCTGGGCGACATCGCAGCACGGGAC
CTGATCTGTGCCCAGAAGTTTAATGGCCTGACCGTGCTGCCACCCCTG
CTGACAGATGAGATGATCGCACAGTACACAAGCGCCCTGCTGGCAGGA
ACCATCACATCCGGATGGACCTTCGGCGCAGGAGCCGCCCTGCAGATC
CCCTTTGCCATGCAGATGGCCTATAGGTTCAACGGCATCGGCGTGACC
CAGAATGTGCTGTACGAGAACCAGAAGCTGATCGCCAATCAGTTTAAC
TCCGCCATCGGCAAGATCCAGGACAGCCTGTCCTCTACAGCCTCCGCC
CTGGGCAAGCTGCAGGATGTGGTGAATCAGAACGCCCAGGCCCTGAAT
ACCCTGGTGAAGCAGCTGAGCTCCAACTTCGGCGCCATCTCTAGCGTG
CTGAATGATATCCTGAGCCGGCTGGACCCCCCCGAGGCAGAGGTGCAG
ATCGACCGGCTGATCACAGGCAGACTGCAGTCTCTGCAGACCTATGTG
ACACAGCAGCTGATCAGGGCAGCAGAGATCAGGGCAAGCGCCAATCTG
GCAGCAACCAAGATGTCCGAGTGCGTGCTGGGCCAGTCTAAGAGAGTG
GACTTTTGTGGCAAGGGCTATCACCTGATGTCCTTCCCTCAGTCTGCC
CCACACGGCGTGGTGTTTCTGCACGTGACCTACGTGCCCGCCCAGGAG
AAGAACTTCACCACAGCCCCTGCCATCTGCCACGATGGCAAGGCCCAC
TTTCCAAGGGAGGGCGTGTTCGTGTCCAACGGCACCCACTGGTTTGTG
ACACAGCGCAATTTCTACGAGCCCCAGATCATCACCACAGACAATACC
TTCGTGAGCGGCAACTGTGACGTGGTCATCGGCATCGTGAACAATACC
GTGTATGATCCACTGCAGCCCGAGCTGGACAGCTTTAAGGAGGAGCTG
GATAAGTACTTCAAGAATCACACCTCCCCTGACGTGGATCTGGGCGAC
ATCAGCGGCATCAATGCCTCCGTGGTGAACATCCAGAAGGAGATCGAC
CGCCTGAACGAGGTGGCCAAGAATCTGAACGAGAGCCTGATCGATCTG
CAGGAGCTGGGCAAGTATGAGCAGTACATCAAGTGGCCATGGTACATC
TGGCTGGGCTTCATCGCCGGCCTGATCGCCATCGTGATGGTGACCATC
ATGCTGTGCTGTATGACATCCTGCTGTTCTTGCCTGAAGGGCTGCTGT
AGCTGTGGCTCCTGCTGTAAGTTTGATGAGGACGATTCCGAACCCGTG
CTGAAGGGAGTGAAGCTGCATTACACCTGA

### DETAILED DESCRIPTION

### I. Abbreviations

Ad adenovirus
CoV coronavirus
COVID-19 coronavirus disease 2019
Env envelope
GI gastrointestinal
HIV human immunodeficiency virus
IFU infection forming units
IM intramuscular
IN intranasal
OPV oral poliovirus
PP double protein substitution
S spike protein
SARS severe acute respiratory syndrome
TT tail truncated
URT upper respiratory tract
VOC variant of concern
Wu Wuhan strain

### II. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Adenovirus: A non-enveloped virus with a liner, double-stranded DNA genome and an icosahedral capsid. There are at least 68 known serotypes of human adenovirus, which are divided into seven species (species A, B, C, D, E, F and G). Different serotypes of adenovirus are associated with different types of disease, with some serotypes causing respiratory disease (primarily species B and C), conjunctivitis (species B and D) and/or gastroenteritis (species F and G). Adenovirus type 4 (Ad4) is a species E virus that can cause acute respiratory disease and ocular disease. Adenovirus-based vectors are commonly used for a variety of therapeutic applications, including vaccine and gene therapy vectors. In some embodiments herein, the adenovirus vector is a human replication-competent Ad4 with a complete or partial deletion in the E3 region.

Adjuvant: A component of an immunogenic composition used to enhance antigenicity. In some embodiments, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC; see also Wegmann, Clin Vaccine Immunol 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007).

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, intranasal, inhalation, oral, injection (such as subcutaneous, intramuscular, intradermal, intraperitoneal, and intravenous), sublingual, rectal, transdermal (for example, topical) and vaginal routes.

Codon-optimized: A nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Conservative variant: A protein containing conservative amino acid substitutions that do not substantially affect or decrease the function of a protein, such as a coronavirus spike protein. “Conservative” amino acid substitutions are those substitutions that do not substantially affect or decrease a function of a protein, such as the ability of the protein to elicit an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of a protein, such as a recombinant Env protein, such as the ability to elicit an immune response when administered to a subject. For instance, if an amino acid residue is essential for a function of the protein, even an otherwise conservative substitution may disrupt that activity. Thus, a conservative substitution does not alter the basic function of a protein of interest.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-COV), SARS-COV-2, and Middle East respiratory syndrome coronavirus (MERS-COV). Other coronaviruses that infect humans include human coronavirus HKU1 (HKU1-COV), human coronavirus OC43 (OC43-CoV), human coronavirus 229E (229E-CoV), and human coronavirus NL63 (NL63-CoV).

COVID-19: The disease caused by the coronavirus SARS-COV-2.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

E3 region: Refers to the adenovirus early region 3 (E3) gene, which contains multiple open reading frames (ORFs). The E3 region of human adenovirus type 4 (Ad4) includes the following ORFs: 12.1K, 23.3K, 19K, 24.8K, 6.3K, 29.7K, 10.4K, 14.5K and 14.7K. In some embodiments herein, the deletion in the E3 region comprises a deletion of the 23.3K, 19K, 24.8K, 6.3K, 29.7K, 10.4K, 14.5K and 14.7K ORFs. In other embodiments, the deletion in the E3 region is a deletion of only the 24.8K, 6.3K and 29.7K ORFs.

Heterologous: Originating from a separate genetic source or species. For example, a heterologous polypeptide or polynucleotide refers to a polypeptide or polynucleotide derived from a different source or species.

Immune response: A response of a cell of the immune system, such as a B cell, T cell, or monocyte, to a stimulus. In some embodiments, the response is specific for a particular antigen (an “antigen-specific response”), such as a SARS-COV-2 spike protein. In some embodiments, the immune response is a T cell response, such as a CD4+ response or a CD8+ response. In other embodiments, the response is a B cell response, and results in the production of specific antibodies. “Priming an immune response” refers to treatment of a subject with a “prime” immunogen/immunogenic composition to induce an immune response that is subsequently “boosted” with a boost immunogen/immunogenic composition. Together, the prime and boost immunizations produce the desired immune response in the subject.

Immunogenic composition: A composition that includes an immunogen or a nucleic acid molecule or vector encoding an immunogen (such as SARS-COV-2 spike protein), that elicits a measurable CTL response against the immunogen, and/or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition can include the protein or nucleic acid molecule in a pharmaceutically acceptable carrier and may also include other agents, such as an adjuvant.

Immunize: To render a subject protected from infection by a particular infectious agent, such as SARS-COV-2. Immunization does not require 100% protection. In some examples, immunization provides at least 50%, at least 60%, at least 70%, at least 80%, at least 90% or at least 95% protection against infection compared to infection in the absence of immunization.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Neutralizing antibody: An antibody that reduces the infectious titer of an infectious agent by binding to a specific antigen on the infectious agent, such as a virus (e.g., a coronavirus). In some embodiments, an antibody that is specific for a SARS-COV-2 spike protein neutralizes the infectious titer of SARS-COV-2. For example, an antibody that neutralizes SARS-COV-2 may interfere with the virus by binding it directly and limiting entry into cells. Alternately, a neutralizing antibody may interfere with one or more post-attachment interactions of the pathogen with a receptor, for example, by interfering with viral entry using the receptor. In some embodiments, a SARS-COV-2 neutralizing antibody inhibits SARS-COV-2 infection of cells, for example, by at least 50%, by at least 60%, by at least 70%, by at least 80% or by at least 90%, compared to a control antibody.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens (such as recombinant Ad4 expressing SARS-CoV-2 S protein) and immunogenic compositions.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular embodiments, suitable for administration to a subject the carrier may be sterile, and/or suspended or otherwise contained in a unit dosage form containing one or more measured doses of the composition suitable to elicit the desired anti-SARS-COV-2 immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in viral load. “Ameliorating” refers to the reduction in the number or severity of signs or symptoms of a disease, such as a coronavirus infection.

Recombinant: A recombinant nucleic acid, vector or virus is one that has a sequence that is not naturally occurring or has a sequence that is made by an artificial combination of two otherwise separated segments of sequence. This artificial combination can be accomplished, for example, by the artificial manipulation of isolated segments of nucleic acids, for example, using genetic engineering techniques.

Replication-competent virus: A virus capable of undergoing genome replication and protein synthesis to produce progeny virus.

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

SARS-COV-2: A coronavirus of the genus betacoronavirus that first emerged in humans in 2019. This virus is also known as Wuhan coronavirus, 2019-nCOV, or 2019 novel coronavirus. The term “SARS-COV-2” includes variants thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2) and omicron (B.1.1.529 and BA lineages). Symptoms of SARS-COV-2 infection include fever, chills, dry cough, shortness of breath, fatigue, muscle/body aches, headache, new loss of taste or smell, sore throat, nausea or vomiting, and diarrhea. Patients with severe disease can develop pneumonia, multi-organ failure, and death. The time from exposure to onset of symptoms is approximately 2 to 14 days. The SARS-COV-2 virion includes a viral envelope with large spike glycoproteins. The SARS-COV-2 genome, like most coronaviruses, has a common genome organization with the replicase gene included in the 5′-two thirds of the genome, and structural genes included in the 3′-third of the genome. The SARS-COV-2 genome encodes the canonical set of structural protein genes in the order 5′-spike (S)-envelope (E)-membrane (M) and nucleocapsid (N)-3′.

SARS Spike (S) protein: A class I fusion glycoprotein initially synthesized as a precursor protein of approximately 1256 amino acids for SARS-COV, and 1273 amino acids for SARS-COV-2. Individual precursor S polypeptides form a homotrimer and undergo glycosylation within the Golgi apparatus as well as processing to remove the signal peptide, and cleavage by a cellular protease between approximately position 679/680 for SARS-COV, and 685/686 for SARS-COV-2, to generate separate S1 and S2 polypeptide chains, which remain associated as S1/S2 protomers within the homotrimer, thereby forming a trimer of heterodimers. The S1 subunit is distal to the virus membrane and contains the receptor-binding domain (RBD) that is believed to mediate virus attachment to its host receptor. The S2 subunit is believed to contain the fusion protein machinery, such as the fusion peptide. S2 also includes two heptad-repeat sequences (HR1 and HR2) and a central helix typical of fusion glycoproteins, a transmembrane domain, and a cytosolic tail domain. An exemplary wild-type (Wuhan strain) SARS-COV-2 spike protein sequence is set forth herein as SEQ ID NO: 2. Exemplary modified Wuhan SARS-COV-2 spike protein sequences are set forth herein as SEQ ID NOs: 3-5. In addition, exemplary SARS-COV-2 variant spike protein sequences are set forth herein as SEQ ID NOs: 7-12.

Subject: Living multicellular vertebrate organisms, a category that includes human and non-human mammals. In some embodiments, the subject is a human. In some examples, a subject who is in need of inhibiting or preventing a SARS-COV-2 infection is selected. For example, the subject can be uninfected and at risk of SARS-COV-2 infection.

Therapeutically effective amount: A quantity of a specific substance, such as a disclosed immunogen (e.g., a recombinant Ad4 expressing SARS-COV-2 S protein) or immunogenic composition, sufficient to achieve a desired effect in a subject being treated, such as a protective immune response. A “therapeutically effective amount” can be the amount necessary to inhibit SARS-COV-2 replication or treat COVID-19 in a subject with an existing SARS-COV-2 infection. A “prophylactically effective amount” refers to administration of an agent or composition that inhibits or prevents establishment of an infection, such infection by SARS-COV-2. It is understood that to obtain a protective immune response against an antigen of interest, multiple administrations of a disclosed immunogen/immunogenic composition can be required, and/or administration of a disclosed composition as the “prime” in a prime boost protocol wherein the boost immunogen can be different from the prime immunogenic composition. Accordingly, an effective amount of a disclosed immunogen/immunogenic composition can be the amount of the immunogen or immunogenic composition sufficient to elicit a priming immune response in a subject that can be subsequently boosted with the same or a different immunogen to elicit a protective immune response.

In one example, a desired response is to elicit an immune response that inhibits or prevents SARS-COV-2 infection. The SARS-COV-2 infected cells do not need to be completely eliminated or prevented for the composition to be effective. For example, administration of an effective amount of an immunogen or immunogenic composition can elicit an immune response that decreases the number of SARS-COV-2 infected cells (or prevents the infection of cells) by a desired amount, for example, by at least 50%, at least 60%, at least 70%, at least 80%, at least 90%, at least 95%, at least 98%, or even at least 100% (elimination or prevention of detectable SARS-COV-2 infected cells), as compared to the number of SARS-COV-2 infected cells in the absence of the immunization.

Unit dosage form: A physically discrete unit, such as a capsule, tablet, or solution, that is suitable as a unitary dosage for a human patient, each unit containing a predetermined quantity of one or more active ingredient(s) calculated to produce a therapeutic effect, in association with at least one pharmaceutically acceptable diluent or carrier, or combination thereof.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine reduces the severity of the symptoms associated with SARS-COV-2 infection and/or decreases the viral load compared to a control. In another non-limiting example, a vaccine reduces SARS-COV-2 infection and/or transmission compared to a control.

Vector: An entity containing a DNA or RNA molecule bearing a promoter(s) that is operationally linked to the coding sequence of a protein (such as an immunogenic protein) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. Non-limiting examples of viral vectors include adenovirus vectors, adeno-associated virus (AAV) vectors, and poxvirus vectors (e.g., vaccinia, fowlpox).

### III. Introduction

Of the available vaccine platforms for presenting viral glycoproteins to the immune system, replicating vectors have several important advantages over most non-replicating vectors (Robert-Guroff, Curr Opin Biotechnol 18(6):546-556, 2007). Replication-competent vectors can express viral surface proteins such that the total dose of antigen vastly exceeds those of non-replicating vectors. Replicating mucosal vaccines induce mucosal immunity, including IgA and IgG antibodies, and a balanced T cell response including resident memory T cells. In addition, replicating vectors, such as replication-competent adenovirus (Ad) vectors, express viral glycoproteins over a prolonged period of time, similar to live virus infections. This feature is thought to be important for the loading of dendritic cells in the lymph node and the induction of a durable antibody response (Cirelli et al., Cell 177(5): 1153-1171, 2019; Tam et al., Proc Natl Acad Sci USA 113(43): E6639-E6648, 2016; Mueller et al., Mol Pharm 12(5): 1356-1365, 2015). Each of these features contributes to the magnitude and durability of immune responses observed after replicating viral vaccinations.

The vaccine constructs disclosed herein are replication-competent Ad4 encoding a SARS-CoV-2 spike (S) protein. In the disclosed Ad4 vector, which is derived from an Ad4 vaccine strain, the gene encoding a SARS-COV-2 spike protein is cloned into an E3 region having a deletion of multiple E3 ORFs. The parent Ad4 vaccine vector has been given to over 10 million people with an excellent safety record. Ad4-recombinants have been developed for both influenza virus H5 and human immunodeficiency virus (HIV) envelope (Env) and Gag proteins. These Ad4-based vaccines have been through pre-clinical testing in rabbits for immunogenicity and human testing in phase 1 clinical trials.

The replication-competent Ad4-based vaccine platform has several distinct advantages compared to other proposed and licensed SARS-COV-2 vaccines. For example, the efficacy of Ad4 vaccines has already been established as they have been administered routinely as a single dose enteric capsule in the U.S. military and found to prevent respiratory disease with an efficacy of greater than 95%. In addition, when administered intranasally or onto the tonsils, replication-competent Ad4-based vaccines induce a neutralizing antibody response in human subjects. Upper respiratory tract administration also bypasses pre-existing Ad4 immunity in most people. By inducing mucosal immunity, the Ad4-based vaccine platform not only provides protection for vaccinated subjects, but also has the potential to interrupt transmission of SARS-COV-2 to others. In contrast to non-replicating viral vaccines, the replication-competent Ad4-based system produces a durable immune response. Furthermore, unlike mRNA-based SARS-COV-2 vaccines, Ad4 vaccines can be stored long term at 4-8° C. Moreover, the disclosed vaccine platform is unmatched in terms of scalability and cost. It is estimated that the disclosed SARS-COV-2 vaccine can be produced for less than 1 cent per dose.

### IV. Overview of Embodiments

Disclosed herein is a recombinant adenovirus type 4 (Ad4) expressing a SARS-COV-2 spike (S) protein (in some embodiments, referred to herein as “Ad4-SARS-COV-2-spike” or “Ad4-Spike”), a recombinant Ad4 nucleic acid vector encoding the recombinant Ad4-Spike, and immunogenic compositions thereof.

In one aspect, provided herein is a recombinant Ad4 expressing a SARS-COV-2 S protein. The recombinant Ad4 is replication-competent and the genome of the Ad4 includes a deletion in the adenovirus E3 region and an insertion of a coding sequence for the SARS-COV-2 S protein. In some embodiments, the amino acid sequence of the S protein is at least 80%, at least 85%, at least 90%, at least 95%, at least 96%, at least 97%, at least 98% or at least 99% identical to the amino acid sequence of a native S protein, such as the S protein of the Wuhan SARS-COV-2 strain set forth herein as SEQ ID NO: 2. In specific examples, the amino acid sequence of the S protein comprises or consists of SEQ ID NO: 2.

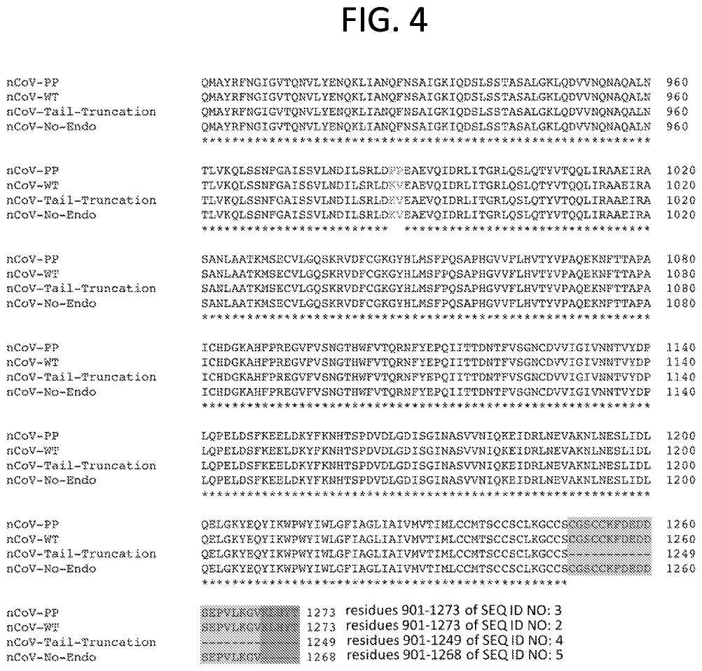

The amino acid numbering used herein for residues of the SARS-COV-2 S protein is with reference to the wild-type Wuhan strain SARS-COV-2 S sequence provided as SEQ ID NO: 2. With reference to the SARS-COV-2 S protein sequence provided as SEQ ID NO: 2, the ectodomain of the SARS-COV-2 S protein includes about residues 16-1208. Residues 1-15 are the signal peptide, which is removed during cellular processing. The S1/S2 cleavage site is located at position 685/686. The HR1 is located at about residues 915-983. The central helix is located at about residues 988-1029. The HR2 is located at about 1162-1194. The C-terminal end of the S2 ectodomain is located at about residue 1208. The position numbering of the S protein may vary between SARS-COV-2 stains, but the sequences can be aligned to determine relevant structural domains and cleavage sites (see, e.g., FIG. 4).

In some embodiments, the recombinant Ad4 comprises a coding sequence for a SARS-CoV-2 S protein comprising one or more (such as two, for example two consecutive) proline substitutions at or near the boundary between a HR1 domain and a central helix domain that stabilize the S protein in the prefusion conformation. In some such embodiments, the one or more (such as two, for example two consecutive) proline substitutions that stabilize the S protein in the prefusion conformation are located between a position 15 amino acids N-terminal of a C-terminal residue of the HR1 and a position 5 amino acids C-terminal of a N-terminal residue of the central helix. In some embodiments, the one or more (such as two, for example two consecutive) proline substitutions that stabilize the SARS-COV-2 S protein in the prefusion conformation are located between residues 975 to 995 (such as 981-992). In some embodiments, the SARS-COV-2 S protein is stabilized in the prefusion conformation by K986P and V987P substitutions (“PP” or “2P”). In some embodiments, the SARS-COV-2 S protein is stabilized in the prefusion conformation by one or two proline substitutions at positions D985, K986, or V987 of the S ectodomain protomers in the trimer. In some examples, the SARS-COV-2 S protein stabilized in the prefusion conformation by the one or more proline substitutions (such as K986P and V987P substitutions) comprises one or more additional modifications for stabilization in the prefusion conformation.

In some embodiments, the SARS-COV-2 S protein encoded by the recombinant Ad4 genome comprises an amino acid sequence at least 90% (such as at least 95%, at least 96%, at least 97%, at least 98%, or at least 99%) identical to SEQ ID NO: 3 (Wuhan-PP), wherein the SARS-CoV-2 S protein is stabilized in the prefusion conformation with one or more of the modifications provided herein (such as the K986P and V987P substitutions). In other embodiments, the stabilized, proline substituted S protein is derived from a SARS-COV-2 variant. In some examples, stabilized S protein derived from a SARS-COV-2 variant comprises an amino acid sequence at least 90% (such as at least 95%, at least 96%, at least 97%, at least 98%, or at least 99%) identical to SEQ ID NO: 7 (beta-PP), SEQ ID NO: 8 (Wuhan/RDB-beta-PP), SEQ ID NO: 9 (delta-PP), SEQ ID NO: 10 (gamma-PP), SEQ ID NO: 11 (delta plus-PP) or SEQ ID NO: 12 (omicron-PP). In particular examples, the amino acid sequence of the stabilized SARS-COV-2 S protein comprises or consists of SEQ ID NO: 3, SEQ ID NO: 7, SEQ ID NO: 8, SEQ ID NO: 9, SEQ ID NO: 10, SEQ ID NO: 11 or SEQ ID NO: 12.

In other embodiments, the SARS-COV-2 S protein encoded by the recombinant Ad4 genome comprises a C-terminal truncation, such as a truncation of the cytoplasmic tail or a truncation of the endocytosis motif. In specific examples, the truncated SARS-COV-2 S protein comprises or consists of the amino acid sequence of SEQ ID NO: 4 or SEQ ID NO: 5.

An exemplary nucleic acid sequence encoding a SARS-COV-2 S protein is provided as SEQ ID NO: 6. In some examples, the nucleic acid sequence encoding the S protein is at least 80%, at least 85%, at least 90%, at least 95%, at least 96%, at least 97%, at least 98% or at least 99% identical to SEQ ID NO: 6. In specific non-limiting examples, the nucleic acid sequence encoding the S protein comprises or consists of SEQ ID NO: 6.

The DNA sequence of the exemplary SARS-COV-2 S protein provided above can be modified to introduce the amino acid substitutions and deletions disclosed herein for prefusion stabilization. In some embodiments, this DNA sequence (with or without modification to introduce amino acid substitutions) can be included in the recombinant Ad4 vector as the sequence encoding the SARS-COV-2 S protein. In some embodiments, the S protein is encoded by a codon-optimized nucleic acid sequence. In some examples, the nucleic acid sequence encoding the S protein is at least 80%, at least 85%, at least 90%, at least 95%, at least 96%, at least 97%, at least 98% or at least 99% identical to SEQ ID NO: 13 (beta-PP), SEQ ID NO: 14 (Wuhan/RBD beta-PP), SEQ ID NO: 15 (delta-PP), SEQ ID NO: 16 (gamma-PP), SEQ ID NO: 17 (delta plus-PP), SEQ ID NO: 18 (omicron-PP) or SEQ ID NO: 19 (Wuhan-PP). In specific examples, the nucleic acid sequence encoding the S protein comprises or consists of any one of SEQ ID NOs: 13-19.

In some embodiments, the deletion in the E3 region is a deletion of at least two, at least three, at least four, at least five, at least six, or at least seven E3 open reading frame (ORFs). In some examples, the deletion includes at least two, at least three, at least four, at least five, at least six, or at least seven of the 23.3K, 19K, 24.8K, 6.3K, 29.7K, 10.4K, 14.5K and 14.7K ORFs. In particular non-limiting examples, the deletion in the E3 region includes a deletion of each of the 23.3K, 19K, 24.8K, 6.3K, 29.7K, 10.4K, 14.5K and 14.7K ORFs.

In some embodiments, the coding sequence for the SARS-COV-2 S protein is inserted in place of the deleted portion of the E3 region.

In some embodiments, the nucleotide sequence of the genome of the recombinant Ad4 is at least 80%, at least 85%, at least 90%, at least 95%, at least 96%, at least 97%, at least 98% or at least 99% identical to SEQ ID NO: 1. In some examples, the nucleotide sequence of the genome of the recombinant Ad4 comprises or consists of SEQ ID NO: 1.

Also provided herein is a recombinant, replication-competent Ad4 nucleic acid vector. In some embodiments, the recombinant Ad4 vector includes a deletion in the adenovirus E3 region and an insertion of a coding sequence for the SARS-COV-2 S protein. In some embodiments, the amino acid sequence of the S protein is at least 80%, at least 85%, at least 90%, at least 95%, at least 96%, at least 97%, at least 98% or at least 99% identical to the amino acid sequence of a native S protein, such as the S protein of the Wuhan SARS-COV-2 strain set forth herein as SEQ ID NO: 2. In specific examples, the amino acid sequence of the S protein comprises or consists of SEQ ID NO: 2.

In some embodiments, the SARS-COV-2 S protein is stabilized in the prefusion conformation by K986P and V987P substitutions (“PP” or “2P”). In some embodiments, the SARS-COV-2 S protein is stabilized in the prefusion conformation by one or two proline substitutions at positions D985, K986, or V987 of the S ectodomain protomers in the trimer. In some examples, the SARS-COV-2 S protein stabilized in the prefusion conformation by the one or more proline substitutions (such as K986P and V987P substitutions) comprises one or more additional modifications for stabilization in the prefusion conformation.

In some embodiments, the SARS-COV-2 S protein encoded by the recombinant Ad4 nucleic acid vector comprises an amino acid sequence at least 90% (such as at least 95%, at least 96%, at least 97%, at least 98%, or at least 99%) identical to SEQ ID NO: 3 (Wuhan-PP), wherein the SARS-COV-2 S protein is stabilized in the prefusion conformation with one or more of the modifications provided herein (such as the K986P and V987P substitutions). In other embodiments, the stabilized, proline substituted S protein is derived from a SARS-COV-2 variant. In some embodiments, the S protein is encoded by a codon-optimized nucleic acid sequence. In some examples, stabilized S protein derived from a SARS-COV-2 variant comprises an amino acid sequence at least 90% (such as at least 95%, at least 96%, at least 97%, at least 98%, or at least 99%) identical to SEQ ID NO: 7 (beta-PP), SEQ ID NO: 8 (Wuhan/RDB-beta-PP), SEQ ID NO: 9 (delta-PP), SEQ ID NO: 10 (gamma-PP), SEQ ID NO: 11 (delta plus-PP) or SEQ ID NO: 12 (omicron-PP). In particular examples, the amino acid sequence of the stabilized SARS-COV-2 S protein comprises or consists of SEQ ID NO: 3, SEQ ID NO: 7, SEQ ID NO: 8, SEQ ID NO: 9, SEQ ID NO: 10, SEQ ID NO: 11 or SEQ ID NO: 12.

In other embodiments, the SARS-COV-2 S protein encoded by the recombinant Ad4 nucleic acid vector comprises a C-terminal truncation, such as a truncation of the cytoplasmic tail or a truncation of the endocytosis motif. In specific examples, the truncated SARS-COV-2 S protein comprises or consist of the amino acid sequence of SEQ ID NO: 4 or SEQ ID NO: 5.

In some embodiments of the disclosed Ad4 vector, the deletion in the E3 region is a deletion of at least two, at least three, at least four, at least five, at least six, or at least seven E3 ORFs. In some examples, the deletion includes at least two, at least three, at least four, at least five, at least six, or at least seven of the 23.3K, 19K, 24.8K, 6.3K, 29.7K, 10.4K, 14.5K and 14.7K ORFs. In particular non-limiting examples, the deletion in the E3 region includes a deletion of each of the 23.3K, 19K, 24.8K, 6.3K, 29.7K, 10.4K, 14.5K and 14.7K ORFs.

In some embodiments of the disclosed Ad4 vector, the coding sequence for the SARS-COV-2 S protein is inserted in place of the deleted portion of the E3 region. In some examples, the coding sequence for the S protein is at least 80%, at least 85%, at least 90%, at least 95%, at least 96%, at least 97%, at least 98% or at least 99% identical to any one of SEQ ID NOs: 2-5 and 7-12. In specific non-limiting examples, the coding sequence for the S protein comprises or consists of any one of SEQ ID NOs: 2-5 and 7-12.

In some embodiments, the nucleotide sequence of the Ad4 vector is at least 80%, at least 85%, at least 90%, at least 95%, at least 96%, at least 97%, at least 98% or at least 99% identical to SEQ ID NO: 1. In some examples, the nucleotide sequence of the Ad4 vector comprises or consists of SEQ ID NO: 1.

Further provided herein are immunogenic compositions that include a recombinant Ad4 or a recombinant Ad4 vector, and a pharmaceutically acceptable carrier. In some embodiments, the immunogenic composition further includes an adjuvant. In other embodiments, the immunogenic composition does not include an adjuvant.

Methods of eliciting an immune response against SARS-COV-2 in a subject are also provided. In some embodiments, the method includes administering to the subject a therapeutically effective amount of a recombinant Ad4, a recombinant Ad4 (nucleic acid) vector, or an immunogenic composition disclosed herein. Also provided are methods of immunizing a subject against SARS-COV-2 infection. In some embodiments, the method includes administering to the subject a therapeutically effective amount of a recombinant Ad4, a recombinant Ad4 vector, or an immunogenic composition disclosed herein.

In some embodiments of the disclosed methods, the recombinant Ad4, recombinant Ad4 vector, or immunogenic composition is administered intranasally or onto the tonsils. In some examples, intranasal administration includes administration of an aerosol. The particle size of the aerosol should allow for delivery to the upper respiratory tract, but not the lower respiratory tract. In specific examples, the aerosol contains particles greater than 10 microns in diameter, such as greater than 20 microns, greater than 30 microns, greater than 40 microns or greater than 50 microns. In particular examples, the aerosol contains particles of about 10 to about 150 microns, such as about 20 to about 125 microns or about 30 to about 100 microns. One of skill in the art is capable of selecting an appropriate device for intranasal delivery of the disclosed recombinant Ad4, recombinant Ad4 vector, or immunogenic composition to the upper respiratory tract. Non-limiting examples of devices include Accuspray™ (Becton-Dickinson) and the MAD Nasal™ (Teleflex®) atomizer.

In some embodiments, the method includes administering a dose of about 10⁴ to about 10⁶ recombinant Ad4 particles, such as about 5×10⁴ to about 5×10⁵ viral particles or about 1×10⁵ viral particles. In some examples, the dose is about 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, or 1×10⁶ recombinant Ad4 particles.

In some embodiments, the recombinant Ad4, the recombinant Ad4 vector, or the immunogenic composition is administered in a single dose.

In some embodiments, the recombinant Ad4, the recombinant Ad4 vector, or the immunogenic composition is administered as part of a prime-boost immunization protocol. In some examples, the recombinant Ad4, the recombinant Ad4 vector, or the immunogenic composition is the prime dose. In other examples, the recombinant Ad4, the recombinant Ad4 vector, or the immunogenic composition is the boost dose.

### V. Preclinical and Clinical Studies Relevant to COVID-19 Vaccine Development

By studying the vaccine-induced mucosal neutralizing antibody responses in a series of live oral poliovirus (OPV) challenge studies, investigators have robustly demonstrated the remarkable separation of the systemic and mucosal antibody systems (Brickley et al., Clin Infect Dis. 2018; 67(suppl_1):S42-S50). This research demonstrates that, despite inducing high levels of serum antibody and providing individual protection from paralytic polio, inactivated Salk vaccines fail to induce the intestinal IgA responses that are critical for inhibiting enteric poliovirus replication and preventing fecal-oral transmission. In contrast, primary vaccination with live attenuated Sabin OPV induces robust mucosal IgA responses and sterilizing immunity upon challenge with live OPV. This observation emphasizes the critical nature of inducing mucosal immunity to prevent infection and transmission of COVID-19. It is believed that the lack of mucosal immunogenicity seen with OPV will be echoed by subunit or replication-incompetent systemically administered SARS-COV-2 vaccines.

In pre-clinical testing of SARS-COV-2 vaccines, a similar advantage to mucosal immunization in blocking infection has been observed. In ferrets, IM or mucosal immunization with a replication-defective Ad5-spike recombinant induced similar levels of spike-specific antibodies in the serum, yet only mucosal immunization induced sterilizing protection of the upper respiratory tract (URT) (Wu et al., Nat Commun 11(1): 4081, 2020). A similar advantage of intranasal administration over intramuscular administration in inducing mucosal immunity and sterilizing protection of the URT has been observed using lentiviral- or chimp adenoviral-spike recombinants in mouse models permissive to SARS-COV-2 infection (Ku et al., Cell Host Microbe S1931-3128(20)30672-7, 2020; Hassan et al., Cell 183(1): 169-184, 2020; King et al., King et al., bioRxiv 2020.10.10.331348, 2020). It has been observed that local specific IgA is highly associated with terminating viral shedding in humans after challenge with coronavirus 229E (Callow et al., J Hyg 95(1): 173-189, 1985).

Prior attempts to protect against a viral mucosal infection for which the host is naïve using a parenterally administered non-replicating vaccine have failed or produced enhanced disease. Examples include respiratory syncytial virus (RSV), parainfluenza virus (PIV)-3, Ad4, rotavirus, and measles virus. The reasons for these failures lie in part in the difficulty in protecting mucosal surfaces coated on their apical surfaces with viral receptors, 100-1000-fold lower antibodies on these surfaces compared to serum, and distorted and short-lived immune responses generated by non-replicating vectors. Clinical trials of the disclosed Ad4-SARS-COV-2-spike vaccine will evaluate in detail the humoral and mucosal responses to the SARS-COV-2 spike protein and the adenovirus vector. It is expected that the disclosed Ad4-SARS-COV-2-spike vaccine will produce mucosal antibodies in the respiratory tract and most closely mimic the immune profile observed following natural SARS-COV-2 infection. Furthermore, it is believed that the disclosed vaccine offers the best possibility for durably interrupting transmission during the COVID-19 pandemic.

Among the recombinant viral vectors available for human use, replicating adenoviruses offer several important advantages. Replicating Ad4 has been given to more than 10 million people in the military as a vaccine against Ad4 respiratory disease and has an extraordinary safety and efficacy record (Gaydos and Gaydos, Mil Med. 1995; 160(6):300-304). This recombinant Ad4 is attenuated by administration to the gastrointestinal tract in the form of an enteric coated tablet, and does not cause respiratory disease (Choudhry et al., Vaccine 2016:34(38) 4558-4564). Using an enteric capsule delivery, a phase 3 study was undertaken with 4,000 volunteers entering basic military training. The results demonstrated a vaccine efficacy of 99.3% and seroconversion in 94.5% against respiratory disease caused by Ad4 (Kuschner et al., Vaccine 2013:31 2963-2971).

In one trial in humans, replicating recombinant adenoviral vectors expressing influenza virus H5 delivered enterically were only modestly immunogenic. This is most likely related to the attenuation of replication by administration to the gastrointestinal tract (Gurwith et al., Lancet Infect Dis. 2013; 13(3):238-50) coupled with the E3 deletion. The introduction of a large gene such as that coding for the coronavirus spike protein into an adenovirus vector involves the removal of most early (in this case E3) genes and conveys at least a 10-fold attenuation to the parent adenovirus in tissue culture, chimpanzees, and humans (Lubeck et al., Nat Med. 1997; 3(6):651-8).

In another clinical trial, high and remarkably durable levels of influenza-specific neutralizing antibodies were observed when a replication-competent Ad4 expressing the influenza virus hemagglutinin type 5 Vietnam (Ad4-H5-Vtn) was administered to the URT compared to the gastrointestinal (GI) tract (Matsuda et al., Sci Immunol. 2019; 4(34):eaau2710; Matsuda et al., J Clin Invest 131(5):e140794, 2021). The vaccine delivered into the URT was very safe (nasal congestion or throat discomfort in 25% of participants, none above grade 2) up to a dose of 10⁸. This level of reactogenicity is at approximately the same level as seen in placebos, and with some parenterally administered non-replicating platforms now being tested against SARS-COV-2, and below that of a currently licensed varicella zoster (Shingrix) vaccine. URT administration of adenoviruses to Ad4-seropositive humans did result in reinfection. URT administration uses the difficulties in protecting the upper respiratory tree to its advantage to overcome vector-specific immunity. An example of that is the ability of an adenovirus expressing Ebola glycoprotein to induce protective immunity on Ebola challenge by the intranasal route in adeno-immune primates while no protection was observed after IM administration of the Ebola construct in previously adeno immune animals.

Prior results with Ad4-H5-Vtn and Ad4-HIV recombinants indicated that nearly all human participants developed a response to the transgene. After a single intranasal or tonsillar administration of the vaccine, increases in H5-specific B cells, H5-specific antibody somatic hypermutation, and potency were observed. The vaccines also induced a very durable response. The response to the licensed split influenza vaccine typically wanes by 5-10-fold within 2-6 months following immunization. However, when Ad4-H5-Vtn participants were asked to return for boosting 3-5 years later, neutralizing antibodies were still at the level that one observes at the peak response after immunization with the licensed vaccine. The Ad4-SARS-COV-2-spike vaccine construct disclosed herein could be used to generate mucosal immunity after a systemic vaccination. Alternatively, a subunit vaccine could be administered following immunization with the disclosed vaccine to boost mucosal and systemic antibody, which has been shown to occur with the H5-Vtn vaccine construct.

### VI. Immunogenic Compositions

Immunogenic compositions that include a disclosed immunogen (e.g., a recombinant Ad expressing a SARS-COV-2 S protein, or a recombinant Ad4 nucleic acid vector comprising a SARS-COV-2 S protein coding sequence), and a pharmaceutically acceptable carrier are also provided. Such compositions can be administered to subjects by a variety of administration modes, for example, intranasal, onto the tonsils, inhalation, oral, intramuscular, subcutaneous, intravenous, intra-arterial, intra-articular, intraperitoneal, or parenteral routes. Methods for preparing administrable compositions are described in more detail in such publications as Remingtons Pharmaceutical Sciences, 19^{th} Ed., Mack Publishing Company, Easton, Pennsylvania, 1995.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The pharmaceutical composition may optionally include an adjuvant to enhance an immune response of the host. Suitable adjuvants are, for example, toll-like receptor agonists, alum, AIPO₄, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product. In some embodiments, an adjuvant is not required and is thus not administered with the Ad4-Spike vaccine.

In some embodiments, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which elicits an immune response without significant, adverse side effects. In some examples, the dose is about 1×10⁴ to about 10⁶ viral particles, such as about 5×10⁴ to about 5×10⁵ viral particles or about 1×10⁵ viral particles.

In some embodiments, the composition can be provided in unit dosage form for use to elicit an immune response in a subject, for example, to prevent SARS-COV-2 infection in the subject. A unit dosage form contains a suitable single preselected dosage for administration to a subject, or suitable marked or measured multiples of two or more preselected unit dosages, and/or a metering mechanism for administering the unit dose or multiples thereof. In some examples, the unit dosage is about 1×10⁴ to about 10⁶ viral particles, such as about 5×10⁴ to about 5×10⁵ viral particles. In specific examples, the unit dosage is about 1×10⁵ viral particles.

### VII. Methods of Eliciting an Immune Response

The disclosed immunogens (e.g., a recombinant replication-competent adenovirus expressing a SARS-COV-2 spike protein), polynucleotides and vectors encoding the disclosed immunogens, and compositions including same, can be used in methods of inducing an immune response to SARS-COV-2 to prevent, inhibit (including inhibiting transmission), and/or treat a SARS-COV-2 infection.

Provided herein are methods of eliciting an immune response against SARS-COV-2 in a subject. In some embodiments, the method includes administering to the subject an effective amount of a recombinant adenovirus, adenovirus vector or immunogenic composition disclosed herein. In some examples, the recombinant adenovirus, vector or immunogenic composition is administered intranasally (such as in a spray) or orally (such as by using enteric-coated tablets).

When inhibiting, treating, or preventing SARS-COV-2 infection, the methods can be used either to avoid infection in an SARS-COV-2 seronegative subject (e.g., by inducing an immune response that protects against SARS-COV-2 infection), or to treat existing infection in a SARS-CoV-2 seropositive subject.

To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize SARS-COV-2 infection. These and other routine methods allow the clinician to select patients in need of therapy using the methods and immunogenic compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The disclosed immunogens can be used in coordinate (or prime-boost) immunization protocols or combinatorial formulations. In certain embodiments, novel combinatorial immunogenic compositions and coordinate immunization protocols employ separate immunogens or formulations, each directed toward eliciting an anti-SARS-COV-2 immune response, such as an immune response to SARS-COV-2 spike protein. Separate immunogenic compositions that elicit the anti-SARS-COV-2 immune response can be combined in a polyvalent immunogenic composition administered to a subject in a single immunization step, or they can be administered separately (in monovalent immunogenic compositions) in a coordinate immunization protocol.

In one embodiment, a suitable immunization regimen includes at least two separate inoculations with one or more immunogenic compositions including a disclosed Ad4-Spike with a second inoculation being administered more than about two, about three to eight, or about four weeks following the first inoculation. A third inoculation can be administered several months after the second inoculation, and in specific embodiments, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of SARS-CoV-2 infection, improvement in disease state (e.g., reduction in viral load), or reduction in transmission frequency. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed immunogen can be increased or the route of administration can be changed.

It is contemplated that there can be several boosts, and that each boost can be a different immunogen. It is also contemplated in some examples that the boost may be the same immunogen as another boost, or the prime.

The prime and the boost can be administered as a single dose or multiple doses, for example, two doses, three doses, four doses, five doses, six doses or more can be administered to a subject over days, weeks or months. Multiple boosts can also be given, such one to five, or more. Different dosages can be used in a series of sequential inoculations. For example, a relatively large dose in a primary inoculation and then a boost with relatively smaller doses. The immune response against the selected antigenic surface can be elicited by one or more inoculations of a subject.

In several embodiments, a disclosed immunogen can be administered to the subject simultaneously with the administration of an adjuvant. In other embodiments, the immunogen can be administered to the subject after the administration of an adjuvant and within a sufficient amount of time to elicit the immune response. In other embodiments, no adjuvant is administered.

SARS-COV-2 infection does not need to be completely inhibited for the methods to be effective. For example, elicitation of an immune response to SARS-COV-2 can reduce or inhibit SARS-COV-2 infection by a desired amount, for example, by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-COV-2 infected cells), as compared to SARS-COV-2 infection in the absence of immunization. In additional examples, SARS-COV-2 replication can be reduced or inhibited by the disclosed methods. SARS-CoV-2 replication does not need to be completely eliminated for the method to be effective. For example, the immune response elicited using one or more of the disclosed immunogens can reduce SARS-COV-2 replication by a desired amount, for example, by at least 10%, at least 20%, at least 30%, at least 40%, at least 50%, at least 60%, at least 70%, at least 80%, at least 90%, at least 95%, at least 98%, or even at least 100% (elimination or prevention of detectable SARS-COV-2 replication), as compared to SARS-COV-2 replication in the absence of the immune response.

Following immunization of a subject, serum can be collected from the subject at appropriate time points, frozen, and stored for neutralization testing. Methods to assay for neutralization activity, include, but are not limited to, plaque reduction neutralization (PRNT) assays, microneutralization assays, flow cytometry based assays, single-cycle infection assays, and pseudovirus neutralization assays.

In some embodiments, immunization is achieved by administration of recombinant Ad4 vector DNA. Immunization by nucleic acid constructs is taught, for example, in U.S. Pat. No. 5,643,578 (which describes methods of immunizing vertebrates by introducing DNA encoding a desired antigen to elicit a cell-mediated or a humoral response), U.S. Pat. Nos. 5,593,972 and 5,817,637 (which describe operably linking a nucleic acid sequence encoding an antigen to regulatory sequences enabling expression), and broadly described in Janeway & Travers, Immunobiology: The Immune System In Health and Disease, page 13.25, Garland Publishing, Inc., New York, 1997; and McDonnell & Askari, N. Engl. J. Med. 334:42-45, 1996.

The following examples are provided to illustrate certain particular features and/or embodiments. These examples should not be construed to limit the disclosure to the particular features or embodiments described.

### EXAMPLES

### Example 1: Expression of Wild-Type and Modified SARS-COV-2 Spike Proteins

The following studies evaluated cell-surface expression of wild-type Wuhan strain SARS-CoV-2 spike protein (SEQ ID NO: 2) and three modified versions of the Wuhan strain spike protein: stabilized (PP), tail truncated (TT), and endocytosis motif truncated (no-Endo). PP contains double proline stabilization substitutions at amino acid positions 986 and 987 (SEQ ID NO: 3); TT includes a deletion of the terminal 24 amino acids of the cytoplasmic tail (SEQ ID NO: 4); and no-Endo contains a deletion of the C-terminal endocytosis signaling motif (SEQ ID NO: 5) (see FIG. 4).

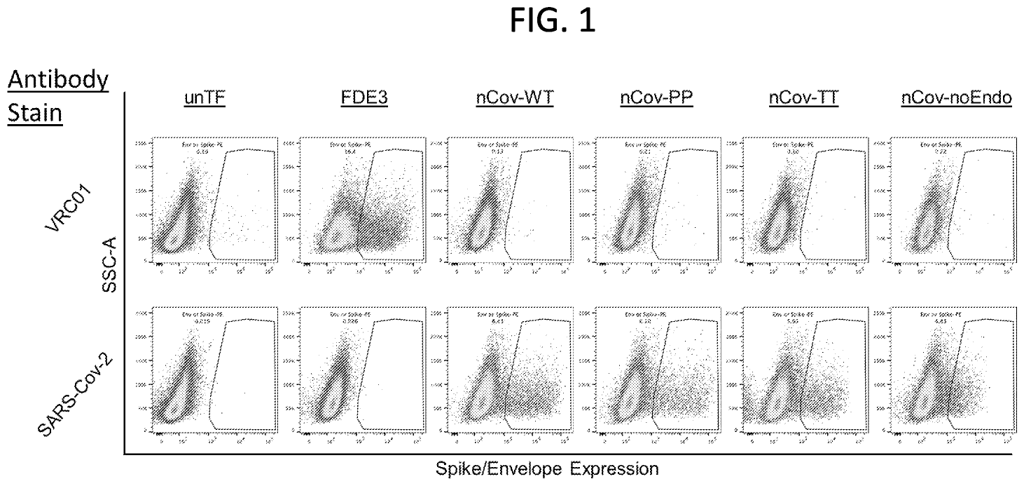

Expression of SARS-COV-2 WT, PP, TT and no-Endo spike proteins was evaluated in A549 cells. Cells were transfected with a shuttle vector plasmid containing the gene for a WT or modified SARS-COV-2 spike protein. Untransfected cells served as negative controls and cells transfected with a plasmid expressing an HIV-1 Env protein was used as a positive control for transfection. Expression of spike and Env was measured by flow cytometry using a SARS-COV-2 spike protein-specific antibody and an HIV Env-specific antibody (VRC01), respectively. As shown in FIG. 1, SARS-COV-2 spike protein expression in transfected A549 cells diminished with truncation of the tail, and truncation of the endocytosis motif, relative to wild-type spike protein.

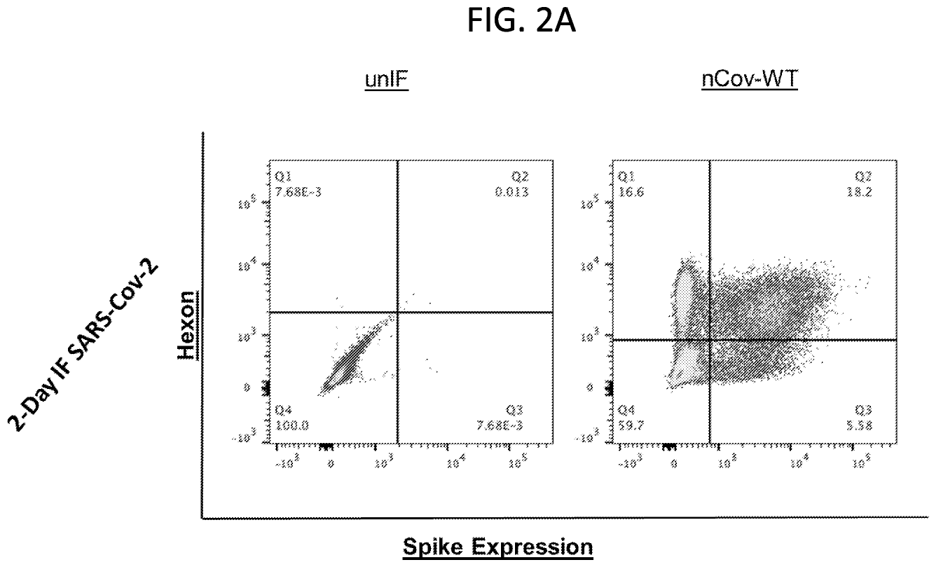

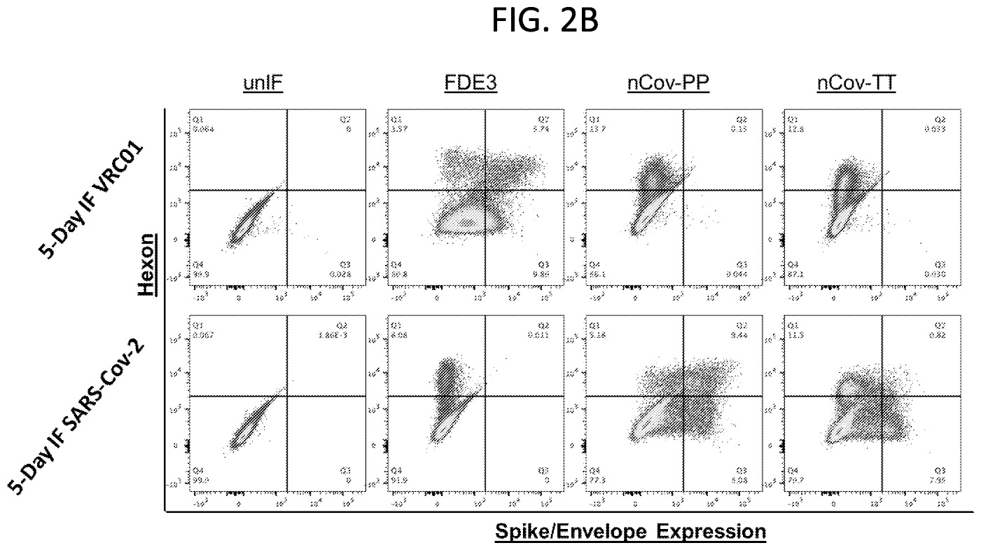

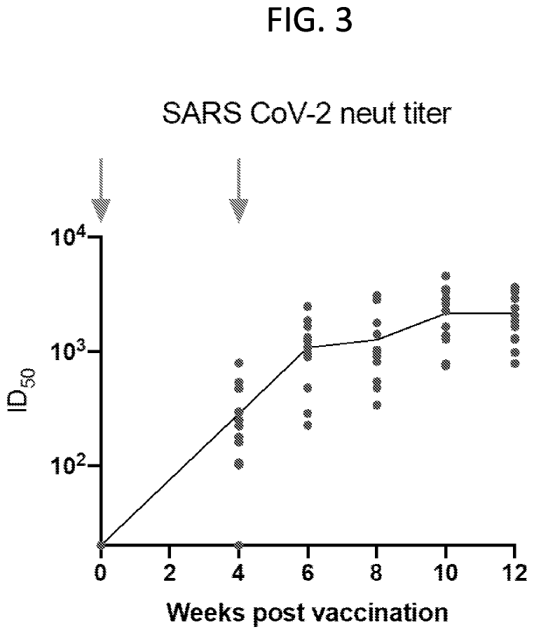

Nucleic acid sequence encoding the WT, PP or TT SARS-COV-2 spike protein was inserted into the E3 region of a replication-competent Ad4 vector having a deletion of the E3 23.3K, 19K, 24.8K, 6.3K, 29.7K, 10.4K, 14.5K and 14.7K ORFs. The nucleotide sequence of the recombinant Ad4 containing the WT spike protein coding sequence is set forth herein as SEQ ID NO: 1. Expression of the WT, stabilized and truncated spike protein in recombinant Ad4-infected A549 cells was evaluated. Replicating Ad4 carrying the WT spike nucleic acid sequence (nCOV-WT), the PP-stabilized spike nucleic acid sequence (nCov-PP) or the tail-truncated spike nucleic acid sequence (nCov-TT) was used to infect A549 cells. A replicating adenovirus expressing an HIV-1 Env protein (FDE3) was used as a positive control of infection and uninfected (unIF) cells were used as a negative control. Expression of spike protein was measured by flow cytometry using a SARS-COV-2 spike protein-specific antibody. Antibody VRC01 was used to detect expression of HIV-1 Env. Spike protein expression from the Ad4-Spike after 2 days of infection is shown in FIG. 2A. In FIG. 2B, expression of the PP-stabilized and truncated Spike proteins is shown. As shown in FIGS. 2A-2B, expression of spike protein was high from both the nCOV-WT and nCoV-PP constructs.

### Example 2: Immunogenicity of Ad4-Spike (WT) in Rabbits

Immunogenicity of Ad4-Spike (expressing the WT spike protein sequence of SEQ ID NO: 2) was tested in New Zealand white rabbits. Rabbits and other experimental animals do not replicate the Ad4 virus, however intramuscular administration (IM) is commonly used as a screen for immunogenicity. Rabbits were immunized IM on day 0 and day 28 with 1.29×10⁹ infectious units (IFU) of purified replicating Ad4-Spike. Using a luciferase assay, serum neutralization against Wuhan SARS-COV-2 pseudovirus was detected at 4 weeks (prior to the second immunization), and continued to increase through the 12-week study period.

### Example 3: Immunogenicity Studies in Hamsters

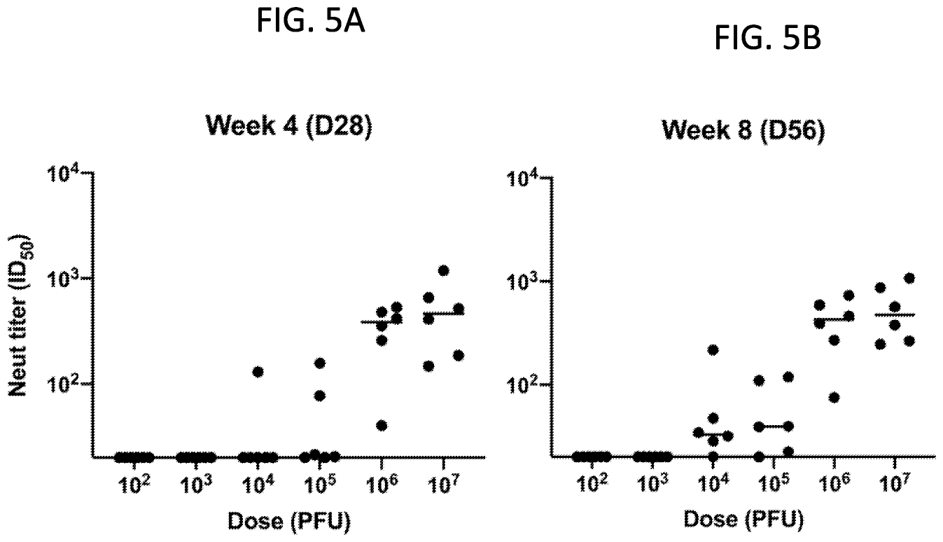

Human adenoviruses are capable of infecting Syrian golden hamsters (van der Lubbe et al., NPJ Vaccines 6(1):39, 2021). Thus, immunogenicity studies were performed in these animals. A dose titration from 102-107 infection forming units (IFU) of intranasal Ad4-SARS-COV-2 Wuhan spike with PP stabilization (Ad4-SARS-COV-2_{WuPP}) was conducted. Strong serum neutralization was observed at week 4 (FIG. 5A) and week 8 (FIG. 5B) in a lentivirus pseudotype assay at the highest doses of Ad4-SARS-COV-2_{WuPP}.

These results suggested that the hamster is semi-permissive for Ad4, but replicates the virus sufficiently to induce serum neutralizing antibodies. Spike-specific IgA and IgG were also observed in the nasal wash on day 60.

Hamsters were then immunized with intranasal Ad4 expressing stabilized (double proline substituted-PP) spike proteins from variants of concern (VOC). Included in this study were Ad4-CoV2-Wuhan, Ad4-CoV2-SA (beta), Ad-CoV2-Wu/RBD-SA, Ad4-CoV2-Indian (delta) and Ad4-CoV2-Brazil (gamma). An Ad4 expressing an influenza virus H5 hemagglutinin (Ad4-H5) and sham inoculation were included as negative controls.

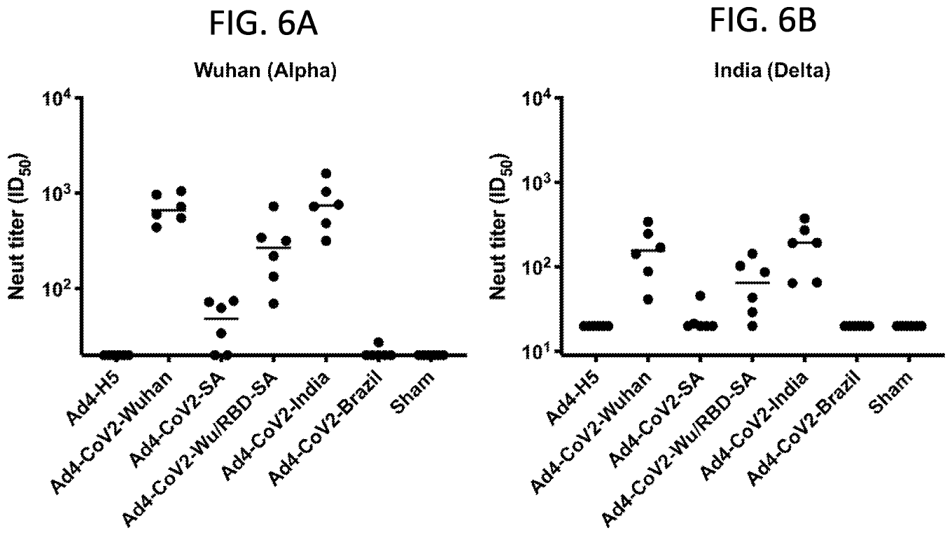

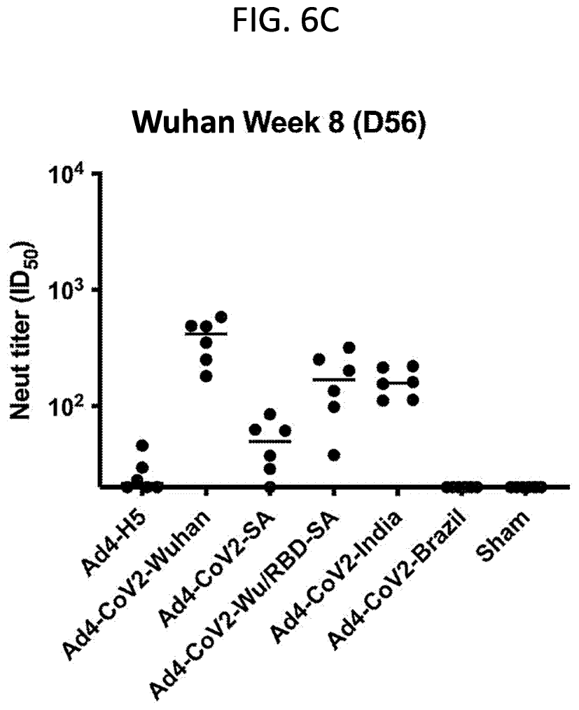

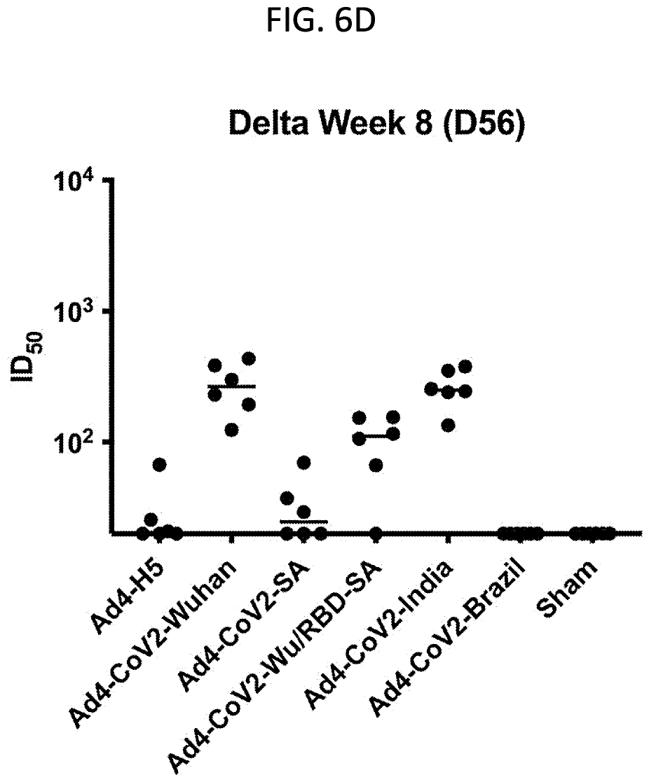

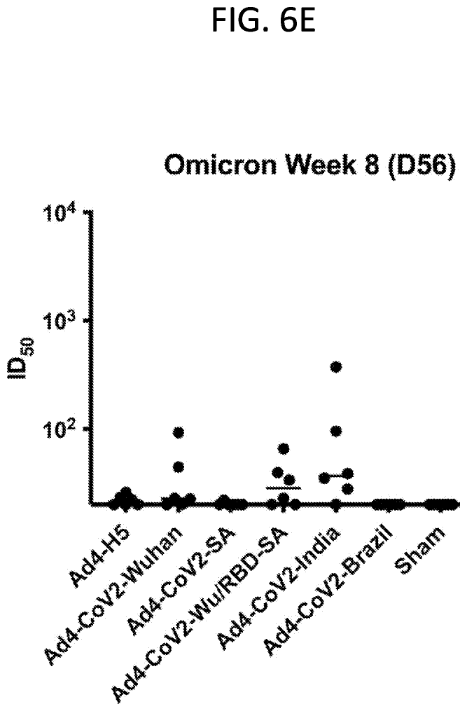

Serum neutralization against Wuhan, delta and omicron pseudovirus was determined 28 days and 56 days following intranasal administration. The results are shown in FIGS. 6A-6E. Ad4 expressing the Wuhan-PP (SEQ ID NO: 3) or Delta-PP (SEQ ID NO: 9) were the most immunogenic.

### Example 4: Challenge Study in Hamsters

This example describes a study to test candidate vaccines in the Syrian golden hamster model.

In this study, Syrian golden hamsters are intranasally administered an immunogenic candidate identified in Example 3 (Candidate 1 or Candidate 2) at a dose of 10⁷ IFU and subsequently challenged with SARS-COV-2 by co-habitation with SARS-COV-2 Delta- or SARS-CoV-2 Omicron-infected animals (van Doremalen et al., Sci Transl Med 13(607):eabh0755, 2021). Table 1 shows the groups of animals that are used. Animals in Group A are challenged at day 60, while animals in Group B are challenged 6 months after immunization. Hamsters receiving intranasal administration of Ad4-H5-Vtn are included as negative controls. Pfizer mRNA or Ad26-Spike is administered intramuscularly as a comparator.

TABLE 1
Challenge study in hamsters

| | Vaccine candidate | Dose |
|---|---|---|
| Group A | | |
| 1a | Ad4-H5-Vtn | 1 × 10⁷ IFU IN |
| 2a | Candidate 1 | 1 × 10⁷ IFU IN |
| 3a | Candidate 2 | 1 × 10⁷ IFU IN |
| 4a | Pfizer mRNA BNT162b2 | 5 μg × 2 IM |
| 5a | Ad26-Spike | 1 × 10⁹ VPU IM |
| Group B | | |
| 1b | Ad4-H5-Vtn | 1 × 10⁷ IFU IN |
| 2b | Variant 1 | 1 × 10⁷ IFU IN |
| 3b | Variant 2 | 1 × 10⁷ IFU IN |
| 4a | Pfizer mRNA BNT162b2 | 5 μg × 2 IM |
| 4b | Ad26-Spike | 1 × 10⁹ VPU IM |

It is expected that intranasal Ad4-Spike vaccine will give systemic neutralizing antibodies that are of the same order of magnitude as mRNA or Ad26 but is more durable. It is also expected that the Ad4-Spike will cause greater restriction of the challenge virus compared to parenterally administered vaccines.

### Example 4: Human Clinical Study

A Phase 1/2 open-label study of a single dose of intranasally administered Ad4-Spike in healthy volunteers is conducted. Enrollment begins with volunteers who may or may not have had prior coronavirus disease 2019 (COVID-19) or vaccination. The international setting chosen is one where supplies of COVID-19 vaccines are limited and SARS-COV-2-naïve volunteers may be more easily enrolled. All SARS-COV-2-naïve participants are offered an emergency use authorization (EUA) vaccine at the completion of the study or following the 6-month timepoint if their neutralization titer is below ˜40 (which is the lower boundary of the interquartile range for the Moderna mRNA 1272 vaccine). Each study participant receives a single dose of an intranasal Ad4-SARS-COV-2 vaccine or an intramuscular (IM) immunization with an authorized or licensed booster. Study participants are monitored for adverse events (AEs), and blood and respiratory secretions are collected for immunogenicity and safety testing periodically throughout the study period. Nasal swabs are collected to monitor adenovirus shedding, and nasal washes are collected to monitor mucosal immune responses. Household and intimate contacts willing to participate are also enrolled and monitored for transmission of the vaccine virus by serology.

The primary endpoints are for safety measured by the frequency and grade of solicited and unsolicited adverse events in the first 28 days after vaccination. Safety is evaluated by separately assessing the incidence, severity, and type of adverse events in the candidate vaccine arms of the trial over the duration of follow-up. It is expected that 21% (N=10/48) of vaccine recipients may experience vaccine-related signs and symptoms (e.g., headache, fatigue, myalgia, rhinorrhea, nausea, diarrhea). Vaccine virus shedding is evaluated by describing the presence, quantity, and duration of shed virus in serially collected nasal wash samples.

A second endpoint is immunogenicity. Immunogenicity is evaluated in serially collected serum, nasal, and stool samples. Immunogenicity is determined by a lentivirus-based pseudovirus neutralization assay. The assay includes functional antibodies as measured by characterization of B-cell clones, complement-enhancement and antibody dependent enhancement, mucosal and T cell immunity. Respiratory mucosal responses are being seen after COVID-19 infection and are thus expected to be a distinguishing hallmark of the Ad4-Spike vaccine. If the Ad4-vectored SARS-CoV-2 vaccine ‘takes’ in 95% of recipients and is immunogenic to adenovirus 4 and SARS-COV-2 spike protein in 90% of these recipients, it is expected that systemic immune responses will be induced in 85% (N=44/52) of vaccine recipients and mucosal responses will be induced in 90-100% of volunteers.

A second dose at 60 days is administered in the rare instance of no evidence of vaccine take at 30 days. However, the primary analysis is after 1 dose as this vaccine is expected to be a single dose regimen. Most participants in prior Ad4-based vaccine trials did not develop a higher response after a second immunization, a second dose would only induce a response in the infrequent case that a participant is not infected on the first dose.

As volunteers will not be pre-screened for serum antibodies, a subset of the volunteers will be seropositive at baseline for Ad4 (˜30%, N=20/60) as a result of exposure to circulating wild-type adenoviruses. The response of those with pre-existing Ad4 immunity in the previous vectored vaccine trials has suggested that Ad4 immunity may modulate the response to the vector and limit virus shedding, but vector specific immunity will still be induced.

Participants are monitored for safety and immunogenicity for one year. The Phase 1 trial optionally includes parallel exploratory arms designed into the clinical trial to permit using Ad4-Spike in conjunction with other SARS-COV-2 Spike immunogens such as DNA, mRNA, or protein vaccines. It is expected that Ad4-Spike will contribute greater durability and mucosal T and B cell responses compared to non-replicating, parenterally administered protein or nucleic acid vaccines.

The target study population excludes only those who may be negatively impacted by respiratory viral infections, such as pregnant women or those with severe immunodeficiencies. The symptoms of recombinant Ad4 vaccination, when they occur, tend to be mild and self-limited. Those persons without difficulties in handling upper respiratory infections should not experience severe symptoms with the Ad4-Spike vaccine. Although pre-existing immunity to Ad4 is not uncommon (30%), it is largely overcome by intranasal vaccination. The degree to which vector-specific immunity is overcome will be assessed and is expected to be a function of the replication of the vaccine virus and the immunogenicity of the spike protein. The prevalence of Ad4 antibodies in persons under 16 is extremely low, making this vaccine a very attractive mode to induce durable immunity in school aged children. The primary endpoints are safety and immunogenicity. Safety is definitively addressed in phase 2 of the trial if the primary endpoint is reached.

When prior Ad4 recombinant virus vaccines were given intranasally, the virus replicated at a low level for 2-4 weeks. However, shedding of the virus detected by viral culture was at a low level and for a median of one day. Participants are counselled to avoid intimate contact for 14 days after vaccination. For these reasons, transmission of the vaccine virus to household or intimate contacts has not been observed. Most vaccines are asymptomatic. However, the most common adverse events (AEs) are throat discomfort and nasal congestion in 25% of participants, none above grade 2. It is expected that a recombinant Ad4 that includes the SARS-COV-2 Spike protein will yield results similar to prior Ad4-based, intranasally administered vaccines.

A phase 3 study and/or challenge study is conducted following phase 2.

In view of the many possible embodiments to which the principles of the disclosed subject matter may be applied, it should be recognized that the illustrated embodiments are only preferred examples of the disclosure and should not be taken as limiting the scope of the disclosure. Rather, the scope of the disclosure is defined by the following claims. We therefore claim all that comes within the scope and spirit of these claims.

1. A recombinant adenovirus type 4 (Ad4) expressing a SARS-COV-2 spike (S) protein, wherein:
the amino acid sequence of the S protein is at least 95% identical to SEQ ID NO: 2;
the recombinant Ad4 is replication-competent; and
the genome of the recombinant Ad4 comprises a deletion in the adenovirus E3 region and an insertion of a coding sequence for the SARS-COV-2 S protein.

2. The recombinant Ad4 of claim 1, wherein the amino acid sequence of the S protein is at least 99% identical to SEQ ID NO: 2.

3. The recombinant Ad4 of claim 1, wherein the amino acid sequence of the S protein comprises or consists of SEQ ID NO: 2.

4. The recombinant Ad4 of claim 1, wherein the amino acid sequence of the S protein comprises at least one modification to stabilize the protein in the prefusion conformation.

5. The recombinant Ad4 of claim 4, wherein the at least one modification comprises K986P and V987P substitutions.

6. The recombinant Ad4 of claim 4, wherein the amino acid sequence of the S protein comprises or consists of SEQ ID NO: 3, SEQ ID NO: 7, SEQ ID NO: 8, SEQ ID NO: 9, SEQ ID NO: 10, SEQ ID NO: 11 or SEQ ID NO: 12.

7. The recombinant Ad4 of claim 1, wherein the deletion in the E3 region comprises a deletion of the 23.3K, 19K, 24.8K, 6.3K, 29.7K, 10.4K, 14.5K and 14.7K open reading frames (ORFs).

8. The recombinant Ad4 of claim 1, wherein the coding sequence for the SARS-COV-2 S protein is inserted in place of the deleted E3 region.

9. The recombinant Ad4 of claim 1, wherein the S protein is encoded by a codon-optimized nucleic acid sequence.

10. The recombinant Ad4 of claim 9, wherein the codon-optimized nucleic acid sequence comprises or consists of SEQ ID NO: 13, SEQ ID NO: 14, SEQ ID NO: 15, SEQ ID NO: 16, SEQ ID NO: 17, SEQ ID NO: 18 or SEQ ID NO: 19.

11. The recombinant Ad4 of claim 1, wherein the nucleotide sequence of the genome is at least 95% identical to SEQ ID NO: 1.

12. (canceled)

13. The recombinant Ad4 of claim 1, wherein the nucleotide sequence of the genome comprises or consists of SEQ ID NO: 1.

14. A recombinant adenovirus type 4 (Ad4) vector, comprising a deletion in the adenovirus E3 region and an insertion of a coding sequence for the SARS-COV-2 S protein, wherein the amino acid sequence of the S protein is at least 95% identical to SEQ ID NO: 2.

15. (canceled)

16. The recombinant Ad4 vector of claim 14, wherein the amino acid sequence of the S protein comprises or consists of SEQ ID NO: 2.

17. The recombinant Ad4 vector of claim 14, wherein the amino acid sequence of the S protein comprises at least one modification to stabilize the protein in the prefusion conformation.

18. The recombinant Ad4 vector of claim 17, wherein the at least one modification comprises K986P and V987P substitutions.

19. The recombinant Ad4 vector of claim 17, wherein the amino acid sequence of the S protein comprises or consists of SEQ ID NO: 3, SEQ ID NO: 7, SEQ ID NO: 8, SEQ ID NO: 9, SEQ ID NO: 10, SEQ ID NO: 11 or SEQ ID NO: 12.

20. The recombinant Ad4 vector of claim 14, wherein the deletion in the E3 region comprises a deletion of the 23.3K, 19K, 24.8K, 6.3K, 29.7K, 10.4K, 14.5K and 14.7K open reading frames (ORFs).

21. The recombinant Ad4 vector of claim 14, wherein the coding sequence for the SARS-COV-2 S protein is inserted in place of the deleted E3 region.

22. The recombinant Ad4 vector of claim 14, wherein the S protein is encoded by a codon-optimized nucleic acid sequence.

23. The recombinant Ad4 vector of claim 22, wherein the codon-optimized nucleic acid sequence comprises of consists of SEQ ID NO: 13, SEQ ID NO: 14, SEQ ID NO: 15, SEQ ID NO: 16, SEQ ID NO: 17, SEQ ID NO: 18 or SEQ ID NO: 19.

24. The recombinant Ad4 vector of claim 14, wherein the nucleotide sequence of the vector is at least 95% identical to SEQ ID NO: 1.

25. (canceled)

26. The recombinant Ad4 vector of claim 14, wherein the nucleotide sequence of the vector comprises or consists of SEQ ID NO: 1.

27. An immunogenic composition comprising the recombinant Ad4 of claim 1, and a pharmaceutically acceptable carrier.

28. A method of eliciting an immune response against SARS-CoV-2 in a subject, comprising administering to the subject a therapeutically effective amount of the recombinant Ad4 of claim 1, thereby eliciting an immune response against SARS-COV-2 in the subject.

29. A method of immunizing a subject against SARS-COV-2 infection, comprising administering to the subject a therapeutically effective amount of the recombinant Ad4 of claim 1, thereby immunizing the subject against SARS-COV-2 infection.

30. The method of claim 28, wherein administration comprises intranasal administration.

31. The method of claim 30, wherein intranasal administration comprises administration of an aerosol comprising particles greater than 10 microns in diameter.

32. The method of claim 28, comprising administering a dose of about 10⁴ to about 10⁶ recombinant Ad4 particles.

33. (canceled)

34. The method of claim 28, wherein the recombinant Ad4 is administered in a single dose.


SEQUENCE LISTING (19 sequences)

SEQ ID NO: 1 - DNA, 40459 bp, Artificial Sequence
  Recombinant vector
taaatttaaatgaattccgtcaagggcgacacaaaaggtattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccaggatgcctggcagttccctactctcgccgctgcgctcggtcgttcggctgcgggacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagcttagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttatcgctgaggatccatttaaatttaattaacatcatcaataatataccttattttttttgtgtgagttaatatgcaaataaggcgtgaaaatttggggatggggcgcgctgattggctgtgacagcggcgttcgttaggggcggggcaggtgacgttttgatgacgcgactatgaggaggagttagtttgcaagttctggtggggaaaagtgacgtcaaacgaggtgtggtttaaacacggaaatactcaattttcccacgctgtctaacaggaaatgaggtgtttttgggcggatgcaagtgaaaacggaccattttcgcgcgaaaactgaatgaggaagtgaaatctgagtaatttagtgtttatgacagggaggagtatttgccgagggccgagtagactttgaccgtttacgtgggggtttcgattaccgtgtttttcacctaaagttccgcgtacggtgtcaaagtccggtgtttttacgtaggtgtcagctgatcgtcagggtatttaaacctgcgctctgcagtcaagaggccactcttgagtgccagcgagaagagttttctcctccgcgccgcgagtcagatctacactttgaaatatgaggcacctaagagacctgcccgatgaggaaattatcatcgcttccgggagcgagattctggaactggtggtaaatgctatgatgggcgacgaccatccggaaccccccaccccatttgagacaccttcgctgcacgatttgtatgatctggaggtggatgtgcccgaggacgaccccaacgagaaggcggtaaatgatttatttagcgatgccgcgctgctagctgccgaggaggcttcaagccctagctcagacagcgactcttcactgcatacccctagacacgacagaggtgagaaagagatccccgggcttaaatgggaaaagatggacttgcgttgctatgaggaatgcctgcccccaagcgatgatgaggacgagcaggcgattcagaacgcagcgagccatggagtgcaagccgtcagcgagagctttgcactggactgcccgcctttgcccggacacggctgtaagtcttgtgaatttcatcgcatcaatactggagataaagctgtgttatgtgcactttgctatatgagagcgtacaaccattgtgtttacagtaagtgtgattaagtgaactttaaagggaggcaaagagtagggtgactgggtgatgactggtttatttatgtatatctgttttttatataggtcccgtttctgacgcagatgatgagacccccactacagagtccactttgtcaccccctgaaattggcacgtctccatctgacaatattgttagaccagttcctgtaagagccactgggaggagagcagctgtagaatgtttggatgatttgcttcagggtggagatgaacctttggacttgtgtacccggaaacgccccaggcattaagtgccacacatgtgtgtttacttgaggtgatgtcagtatttatagggtgtggagtgcaataaaatatgtgttgactttaagtgcgtggtttatgactcaggggaggggactttgggtatataagcaggtgcagacctgtgtggttagctcagagcggtatggagatttggacggttttggaagactttcacaagactaggcagctgctagagaacgcctcgaacggagtctcttacctgtggagattctgcttcggcggtgacctagctaagctagtctatagggccaaacaggattatagggaacaatttgaggatattttgagagagtgtcctggtctttttgacgctcttaacttgggccatcagtctcactttaaccagagaatttcaagagcccttgactttactactcctggcagaaccactgcagcagtagccttttttgcttttatttttgacaaatggagtcaagaaacccatttcagcagggattaccagctggatttcttagcagtagctttgtggagaacatggaagtgccagcgcctgaatgcaatctccggctacttgccggtacagccgctagacactctgaggatcctgagtctccagcagcaggaggatcaagaagagaatccgagagccggcctggaccctccggcggaggagtagctgacctgtttcctgaactgcaccgggtgctgactaggtcttcgagtggtcgggagaggggtattaagcgggagaggcatgatgagactaatcacagaattgaactgactgtgggtctgatgagccgcaagcgtccagaaacagtgtggtggtatgaggtgcagtcaactggcacagatgaggtgtcagtcatgcatgagagattttccctagaacaagtcaagacttgttggttggagcctgaggatgattgggaggtagccatcaggaattatgccaagctggctctgaggccagatagaaagtacaagattactaagctgataaatatcagaaatgcctgctacatctcagggaatggggctgaagtggagatctgtctccaggatagagtggctttcagatgctgcatgatgaatatgtacccgggagtggtggacatggatggggtcacctttatgaacatgaggttcaggggagatgggtataatgggacggtctttatggccaataccaagctgacagtgcatggatgctccttctttgggtttaataacacctgcatcgaggcttggggtcaggtcggtgttaaggggtgcagtttttcagccaactggatgggggtagtgggcaggaccaagagtatgctgtctgtgaagaaatgcttgtttgagaggtgccacctgggggtgatgagcgagggcgaagccagaatccgccactgtgcctctaccgagacgggctgttttgtgctgtgcaagggcaatgccaagatcaagcataatatgatctgtggagcctcggacgagcgcggctaccagatgctgacctgcgccggtgggaacagtcatatgctggccgccgtgcatgtggcttcccattcccgcaagccctggcctgagttcgagcacaatgtcatgaccaggtgcaatatgcatctgggggctcgccgaggcatgtttatgccctaccagtgcaacctgaattatgtaaaggtgctcctggagcccgatgtcatgtccagagtgagcctgacgggggtgtttgacatgaatgtggaagtgtggaagattctaagatatgatgaatacaagaccaggtgtcgagcctgcgagtgcggagggaagcatgccaggttccagcccgtgtgtgtggatgtgacggaggacctgcgacccgatcatttggtgttgtcctgcaccgggacggagttcggctccagtggggaagaatctgactagagtgagtagtgttttggggagggagaggacctgcataaggggcagaatgattaaaatctgtgcttttctgtgtgttgcagcagcatgagcggaaacggctcctttgagggaggggtattcagcccttatctgacggggcgtctcccctcctgggcgggagtgcgtcaaaatgtgatgggatccacggtggacggccggcccgtacagcccgcgaactcttcaaccctgacctatgcaaccctgagctcctcgtcggtggacgcagctgccgccgcagctgctgcttctgccgccagcgccgtgcgcggaatggccatgggcgccggctattacggcactctggtggccaactcgagttccactaataatcccgccagcctgaacgaggagaagctgctgctgttgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaaaaatcagagtctgaatctttatttgatttttcgcgcacggtaggccctggaccaccggcctcgatcattgagcacccggtggatcttttccaagacccggtagaggtgggattggatattgaggtacatgggcatgagcccgtcccgggggtgaaggtagctccattgcagggcctcgtgctcgggggtggtgttgtaaatcacccagtcatagcagggacgcagggcgtggtgttgcacaatatctttgaggaggagactgatggccacgggcagccctttggtgtaggtgtttacaaacctgttgagctgggagggatgcatgcggggggagatgaggtgcatcttagcctggatcttcagattggcgatgttaccgcccagatcccgcctgggattcatgttgtgcaggaccaccagcacggtgtatccggtgcacttggggaatttgtcatgcaacttggaagggaaggcatgaaagaatttggagacgcccttgtggccgcccaggttttccatgcactcatccatgataatggctatgggcccgtgggcggcggcttgggcaaagacgtttcgggggtcggacacatcgtagttgtggtcctgggtgagatcttcataggccattttaatgaatttggggcggagggtgcccgattgggggacgaaggtaccctcaatcccgggggcgtagtttccctcacagatctgcatctcccaggccttaagctccgagggggggatcatgtccacctgcggggcgataaagaaaacggtttccggggcgggggagatgagctgggcggaaagcaggttgcggagtagctgggacttaccgcagccggtggggccgtagataaccccaatgaccggctgcaggtggtagttgagggagacacagctgccgtcctccctaagaaggggggccacctcgttcatcatttggcgcacgtgcatgttctcgcgcaccagttccgccaggagtcgctctccgcccagcgagaggagctcctggagcgaggcgaagtttttcagcggcttgagcccgtcggccatgggcattttggaaagggtctgttgcaggagttccaagcggtcccagagctcggtgatgtgctctacggcatctcgatccagcagacctcctcgtttcgcgggttggggcgactgcgggagtagggcgccagacgatgggcgtccagcgcggccagggtccggtccttccagggtcgcagcgtccgcgtcagggtggtctccgtcacggtaaaggggtgcgcgccgggctgggcgcttgcgagggtgcgcttcaggctcatccggctggtcgagaaccgctcccgatcggcgccctgtgcgtcggccaggtagcaattgaccatgagttcgtagttgagcgcctcggccgcgtggcctttggcgcggagcttacctttggaagtctgcccacaggcgggacagaggagggacttgagggcgtagagcttgggggcgaggaagacggactcgggggcgtaggcgtccgcgccgcagtgggcgcagacggtctcgcactccacgagccaggtgaggtcgggctgattgggatcaaaaaccagttttccgccgttctttttgatgcgtttcttacctctggtctccatgagctcgtgtccccgctgggtgacaaagaggctgtccgtgtccccgtaaaccgactttatgggtcggtcctcgagtgggacgccgcggtcctcgtcgtagaggaaacccgaccactctgagacgaaggcccgggtccaagccagcacgaaggaggccacgtgggagggatagcggtcgttatccaccagcgggtccaccttctccagtgtatgcaaacacatgtccccctcgtccacatccaggaaggtgattggcttgtaagtgtaggccacgtgaccgggggtcccggccgggggggtataaaagggggcgggccgctgctcgtcttcactgtcttccggatcgctgtccaggagcgccagctgttggggtaggtattccctctcaaaggcgggcatgacctccgcactcaggttgtcagtttctagaaacgaggaggatttgatattgacggtgccggcggagatgcctttcaagagcccctcgtccatctggtcagaaaagacaatctttttgttgtcgagtttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggttagctcgtctggcacgattctgacctgccagccccggttatgcagggtgatgaggtcaacgctggtggccacctcgccgcgcaggggctcgttggtccagcagaggcggccgcccttgcgcgagcagaaggggggcagggggtccagcataagctcgtcgggggggtcagcatcgatggtgaagatgcctggcaggaggtcggggtcgaagtagcttatgcaggtgcccagatcgtccagagaagcttgccattcgcgcacggccagcgcgcgctcgtagggactaaggggcgtgccccagggcatggggtgggtgagcgcggaggcgtacatgccgcagatgtcgtagacgtagaggggctcatcaaggatgccaatgtaggtggggtagcagcggcccccgcggatgctggcgcgcacgtagtcatacaactcgtgcgagggggcgaggagcccggctccgagattggcgcggctgggtttttcggcgcggtagacgatctgacggaagatggcgtgggagttggaggagatggtgggtctttggaagatgttgaagtgggcgtggggcaggccgaccgagtcgcggatgaagtgggcgtaggagtcttgcagcttggcgacaagctcggcggtgacgaggacgtccagggcgcagtagtcaagggtctcttggatgatgtcatacttgagctggcccttttgtttccacagctcgcggttgagaaggaactcttcgcggtccttccagtactcttcaagggggaacccgtcctggtcggcacggtaagagcctagcatgtagaactggttaacggccttgtaggcgcagcagcccttctccacggggagggcataggcctgggcggccttgcgcagggaggtgtgcgtgagggcgaaggtgtccctgaccatgacctttaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggctggggcacctcggcccggttgttgatgacctgggcggcgagcacgatctcgtcgaagccgttaatgttgtggcccacaatgtatagttccacgaaccgcgggcggcccttgacgtggggcagtttcttgagctcctcgtaggtgagctcgtcggggtcgctgagcccgtgctgctcgagggcccagtcggcgagatgggggttggcgcggaggaaggaagtccagagatccacggccagggcggtttgcagacgatcccggtactggcggaactgctgacccacggccattttttcgggggtgacgcagtagaaggtgcgggggtcgccgtgccaacggtcccattttagctggagggcgagatcaagggcgagctcaacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgaacattcgtgcttgtgtttatacaagcggccacagtgctcgcaacgctgcacgggatgcacgtgctgcacgagctgtacctgggttcctttgacgaggaatttcagtgggaagtggagtcgtggcgcctgcatctggtgctgtactacgtcgtggtggtcggcctggccctcttctgcctcgatggtggtcatgctgacgagcccgcgcgggaggcaggtccagacctcggcgcgaacgggtcggagagcgaggacgagggcgcgcaggccggagctgtccagggtcctgagacgctgcggagtcaggtcagtgggcagcggcggcgcgcggttgacttgcaggagtttttcaagggcgcgcgggaggtccagatggtacttgatctccaccgcgccgttggtggcgacgtcgatggcttgcagtgtcccgtgcccctggggagtgaccaccgtcccccgtttcttcttggcgggcggaagcggtttggcttccatggttaaaagcggcggcgaggacgcgcgccgggcggtaggggcggctcgggacccggaggcagtggtggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagacagttcgacagaatcaatctcggtatcattgacggcggcctgccgcagaatctcttgcacgtcgcccgagttgtcctggtaggcaatctcggtcatgaactgctcgatctcctcctcctgaaggtctccgcggccggcgcgctccacggtggccgcgaggtcgttggagatgcggcccatgagctgcgagaaggcgttcatgcccgcctcgttccagacgcggctgtaaaccacggcgccctcgggatcgcgggcgcgcatgaccacctgggcgaggttgagctccacgtggcgcgcaaaaaccgcgtagttgcagaggcgctggtagaggtagttgagcgtggtggcaatgtgctcagtgacaaagaagtacataatccagcggcggagcggcatttcgctgacgtcgcccagggcttccaagcgctccatggcctcgtaaaagtccacggcgaagttgaaaaactgggagttgcgtgcagatacggtcaagtcctcctccagaagacggatgagctcggcgatggtggcgcgcacctcgcgctcgaaggctcccgtgagttcctccacttcctcctcttcatccactaacatctcttctacttcctcctcaggcggtggtggcgggggagggggcctgcgtcgccggcggcgcacgggcagacggtcgatgaaacgctcgatggtctcgccgcgccggcgtcgcatggtctcggtgacggcgcgcccgtcctcgcggggtcgcagcgtaaagacgccgccgcgcatctccaggtggcccggggggtccccgttgggcagggagagtgcgctgacgatgcatcttatcaattgccccgtagggactccgcgcaaggacctaagcgtctctagatccacgggatctgaaaaccgttgaacgaaggcttcgagccagtcgcagtcgcaaggtaggctgagcacggtttcttctggcggcggtggggtgtgggcgggggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatacgcgtgagcccaaacccgcgctgcggctggacgagcgccaggtcagcgacgacgcgctcggcgaggatggcctgctggatctgggtgagggtggtctggaagtcgtcaaagtcgacgaagcggtggtaggctccggtgttaatggtgtaggagcagttggccatgacggaccagttgacagtctggtgaccgggccgcgcgagctcgtggtacttgaggcgcgagtaggcgcgcgagtcgaagatgtagtcgttgcaggtgcgcaccaggtactggtagccgatgaggaagtgcggcggcggctggcggtagagcggccatcgctcggtggcgggggcgccgggcgctaggtcctcgagcatggtgcggtggtagccgtagatgtaccttgacatccaggtgatgccggcggcggtggtggaggcgcgagggaactcgcggacgcggttccagatgttgcgcagcggcaggaagtagttcatggtgggcacggtctggcccgtgaggcgcgcgcagtcgttgatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaggcctgcacaaaacctccaggatacggaggcgggtcgttttgcaaatttttggcggtcgaaaaaagctagtaagcgcggaaagcggccgaccgcaatggctcactgccgtagattggagaagaatcgccagggttgcgttgcggtgtgccccggttcgagaccgctcgggtcggccgaattccgcggctaacgagggcgtggctgccccgtcgtttccaagaccccataagccagccgacttctccagttacggagcgagcccctcttttgttttgttttttgccagatgcatcccgtactgcggcagatgcgcccccaccctccaccgcaacagcagccccctcctacgcaacagccggcgcttctgcctccgccccagcagcagcaacttccagccactaccgccgcggccgccgtgagcggggccgggcagagtcagtatgacctggctttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcagcccgtttccacgcggggcgggagctgcggcgcggcctggacagaaagagggtgctgagggacgaggatttcgaggcggacgagctgacggggatcagccctgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccattgtgcagaaccccaccagcaaaccgctgacggcgcagctgttcctggtggtgcagcacagtcgggacaacgagacttttagggaggcgctgctgaatatcaccgagcccgagggccgctggcttctggacctggtgaatattctgcagagcatcgtggtgcaggagcgcgggctgccgctgtccgagaagctggcggccatcaacttttcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcgccgcgcggtaagcgccagcaggcggcgcgagctgagcgatcaggagctgatgcacagcctgcagcgggccctgaccggggccgggaccgagggggagagctactttgacatgggcgcggacctgcactggcagcccagccgccgggtcttggaagccgcggcggtcccttacgtagaagaggtggacgatgaggatgagggcgagtacctggaagactgatggcgcgaccgtatttttgctagatgcagcaacagccacctcctgatcccgcaatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtgctggccatcgtgaacgcgctggttgagaacaaggccattcgcggcgacgaggccgggctggtgtacaacgcactgctggagcgcgtggcccgctacaacagcaccaacgtgcagaccaacctggaccgcatggtgaccgacgtgcgcgaagccgtggcccagcgcgaacggttccaccgcgagtccaacctgggatccatggtggcactgaacgccttcctcagcacgcagcccgccaacgtgccccggggccaggaggactacaccaacttcattagcgccctgcggctaatggtgaccgaggtgccccagagcgaggtgtaccagtcgggcccggactacttcttccagaccagtcgccagggcttgcagaccgtgaacctgagtcaggctttcaagaacttgcagggactgtggggcgtgcaggctccggtcggggaccgcgcgacggtgtcgagcctgctgacgccgaactcgcgcctgctgctgctgctggtggcgcccttcacggacagcggtagtatcaaccgcaactcgtacctgggctacctgattaacctgtaccgcgaggccattggccaggcgcacgtggacgagcagacctaccaggagattacccacgtgagccgcgcccttggccaggacgacccgggcaatctggaagccaccctgaacttcttgctgaccaaccggtcgcagaagatcccgccccagtacgcgctgagcgccgaggaggagcgtatattgagatacgtgcagcaaagtgtgggactgttcctgatgcaggagggggccacccccagcgccgcgctcgacatgaccgcgcgcaacatggagcccagcatgtacgccagtaatcgcccgtttattaataagctgatggactacctgcatcgggcggccgccatgaactctgactatttcaccaacgccatcctgaacccccactggctcccgccgccggggttctacacgggcgagtacgacatgcccgaccccaatgacgggtttctgtgggacgacgtggacagcagcgtgttctccccccgaccgggtgctaacgagcgccccttgtggaagaaagagggcagcgaccggcgcccgtcctcggcgctgtccggccgcacgggtgctgccgcagcggtgcccgaggccgccagtccctttccgagcttgtcactgaacagcgtccgcagtagcgagctgggcaggatcacgcgcccgcgcttgctgggcgaggaggagtacttaaataactcgctgttgaggcccgagcgggagaagaacttccccaataacgggatagagagtctggtggataagatgagccgctggaagacgtacgcgcatgagcacagggacgatccccgggcaacgcagggggccaccagccggggcagtgccgcccgtaaacgccgctggcacgacaggcagcggggactgatgtgggacgatgaggattccgccgacgacagcagcgtgttggacttgggcgggagtggtggtggtaacccgttcgctcacctgcgcccccgcgtcgggcgcctgatgtaaaaagaaaccaaaaataaatggtactcaccaaggccatggcgaccagcgtgcgttcgtttcttctctgttgtatctagtatgatgaggcgtgcgtacccggagggtcctcctccctcgtacgagagcgtgatgcagcaggcaatggcggcggcggcggcgatgcagcccccgctggaggctccttacgtgccaccgcggtacctggcgcctacggaggggcgaaacagcattcgttactcggagctggcacccttgtacgataccacccggttgtacctggtggacaacaagtcggcggacatcgcctcgctgaactaccagaacgaccacagcaactttctgaccaccgtggtgcagaacaacgatttcacccccacggaggccagcacccagaccatcaactttgacgagcgctcgcggtggggcggtcagctgaaaaccatcatgcataccaacatgcccaacgtgaacgagttcatgtacagcaacaagttcaaggcgcgggtcatggtctcccgcaagacccccaacggggtgacagtaggggatgattatgatggtagtcaggatgagctgaaatacgagtgggtggagtttgagctgcccgaaggcaacttctcggtgaccatgaccattgacctgatgaacaacgccatcatcgacaattacttggcagtggggcggcagaacggggtgctggagagcgacatcggcgtgaagttcgacacccggaacttcaggctgggttgggaccccgtgaccgagctggtcatgcccggggtgtacaccaacgaggccttccaccccgacatcgtgctgttgcccggctgcggggtggactttaccgagagccgcctcagtaatatgctgggcatccgcaagaggcagcccttccaggagggtttccagatcatgtacgaggacctggatggaggtaacatccccgcgctcttggatgtcgaggcctatgagaaaagcaaggaggagagcgtcgccgcgtcaaccgcagccgtagccaccgcctctaccgaggtccggggcgataattttgctagcgccgcagcagtggcggcggccaaggctgatgaaaccgaaagtaagatagttattcagccggtggagaaggatagcaaggataggagctacaacgtgctctcggacaagaaaaacaccgcctaccgcagctggtacctggcctacaactatggcgaccacgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcatgcccgtctactccaagagcttcttcaacgagcaggccgtctactcgcagcagctgcgcgccttcacctcgctcacgcacgtcttcaaccgcttccctgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaaaaatgtccattctcatctcgcccagtaataacaccggttggggtctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgctcgcaactacacccccgccgccgcgcccgtctccaccgtggacgccgtcattgacagcgtggtgtccgacgcgcgccggtacgcccgcgccaagagccggcggcggcgcatcgcccggcggcaccgtagcaccaccgccatgcgtgcggcgcgagccttgctgcgcagggccaggcgcacgggacgcagggccatgctcagggcggccagacgcgcggcttcaggcgccagcgccggcaggactcggagacgcgcggccacggcggcggcagcggccatagccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggagaaggatgtccaagcgcaaattcaaggaagagatgctccaggtcatcgcgcctgagatctacggccccgcggcggcggtgaaggatgaaagaaatccccgcaaaatcaagcgggtcaaaaaggacaaaaaggaagaagatgatgtggacgatatggtagagtttgtgcgcgagtttgccccccggaggcgcgtgcagtggcgcgggcggaaagtgcgtccggtgctgagacccggcaccacggtggttttcgcgcctggcgagcggtccggcacgacatccaagcgctcctacgatgaggtgtacggggacgaggatattctcgagcaggcggccgagcgcctgggcgagtttgcttacggcaagcgcaaccgccttgcgcccctgaaggaagaggtggtgtccatcccgctggaccacggcaaccccacgccgagtcttaagcccgtgaccctgcagcaggtgctgccgagcgcggcgccgcgtcggggcttgaagcgcgagggcgaggatgtgtaccccaccatgcagctgatggtgcccaagcgccagaagctggaagacgtgctggagaccatgaaggtggacccggacgtgcagcccgaggtcaaggtgaggcccatcaagcaggtggccccgggccttggcgtgcagaccgtggacatcaagatccccacggagcccatggaaacgcagaccgaggtcgtgaagcccatcaccagcaccatggaggtgcagacggatccttggatgccggcggcgccccgaaaaccccggcgcaagtacggcgcggccagcctgctgatgcccaactacgcgctgcatccttccatcatccccacgccgggctaccgcggcacgcgcttctaccacggctataccggctcccgccgccgcaagaccaccacccgccgccgtcgtcgccgcacagctgcaactcccgctgccgccctggtgcggagagtgtaccgccgcggccgcgcgcctctgaccctgccgcgggcgcgctaccacccgagcattaccatttaactttgccgtcgcctttgcagatatggctctcacatgccgcattcgcgtccccattacgggctaccgaggaagaaaaccgcgccgtagaaggctggcgggaagcgggatgcgccgccacccccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacacttggaaattgtaataaacccgaatggactctgacgctcctggtcctgtgatgtgtttttgtagacagatggaagacatcaatttttcgtccctggctccgcgacacggcacgcggccgtttatgggcacctggagcgacatcggcaccagccaactgaacgggggcgccttcaattggagcagtctctggagcgggcttaagaattttgggtccacgcttaaaacctatggcagcaaggcgtggaacagcaccacagggcaggcgctgagagataagctgaaagagcagaacttccagcagaaggtagtcgatggcctcgcctcaggcatcaacggggtggtggacctggccaatcaggccgtgcagcggcagatcaacagccgcctggacccggttccccccgccggctccgtggagatgccgcaggtggaggaggagctgcctcccctggacaagcggggcgacaagcgtccccgtcccgacgcggaggagacgctgctgacgcacacggacgaaccgcccccgtacgaggaggcggtgaaactgggcctgcccaccacgcgtcccattgcgcctctagctaccggggtgctgaaacccgagagtagtaagcccgcgaccttggacttgcctcctccgcccactccccgcccctccacagtggctaagcccctgccgccggtggccgtggcccgcgcgcgaccgggggctcgccctcaggcgaactggcagagcactctgaacagcatcgtgggtctgggagtgcagagtgtgaagcgccgccgctgttattaaaaaacactgtagcgcttaacttgcttgtctgtgtatatgtgtatgtccgccgccgctgctgtccagaaggaggagtgaagagaaaggcgcgtcgtcgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcctacccacgatgtgaccaccgaccgcagccagcggctgacgctgcgctttgtgcccgtggaccgggaggacaacacctactcgtacaaagtgcgctacacgctggccgtgggcgacaaccgcgtgctggacatggccagcacctactttgacatccgcggcgtgctggatcggggccctagcttcaaaccctactccggcactgcctacaacagcctggctcccaagggagcgcccaacacctgccagtggaaggattctgacagcaaaatgcatacctttggggcagctgccatgcccggtgttactgggaaaaagatagaagctgatgggctgcctattagaatagattcaacttctggaactgacacagtaatttatgctgataaaactttccaaccagaaccacaagttggaaatgacagttgggttgacaccaatggtgcagaggaaaaatatggaggcagagctctaaaggacactacaaaaatgaaaccctgttatggttcattcgccaagcctaccaacaaagaaggtggtcaggctaacttaaaagattcagaacccgccgccaccactcctaactatgatatagacctggctttctttgacagcaaaactattgttgctaactacgatccagatattgtaatgtacacagaaaatgttgacttgcagactccagatactcatattgtatacaaacctggaacagaggacaccagctctgaatccaatttgggtcagcaggccatgcctaacagacccaactacattggcttcagagacaattttatcgggctcatgtactacaacagcactggcaatatgggggtgctggccggtcaggcctctcagctgaatgctgtggttgacttgcaagacagaaacactgaactgtcctaccagctcttgcttgactctctgggtgacagaacccggtatttcagtatgtggaatcaggcggtggacagctatgatcctgatgtgcgcattattgaaaaccatggtgtggaggatgaattgccaaactattgctttccgttgaatggtgtgggattgacagacacttaccagggtgttaaagttaaaacagatgcaggttctgaaaagtgggacaaagatgacaccacagttagtaatgctaatgaaatccatgtaggcaatccttttgccatggaaatcaacatccaagccaacctgtggaggaacttcctctatgccaatgttgccctctatttgcctgataaatacaaatacacaccggccaacatcaccctgcccaccaacaccaacacctacgagtacatgaacggccgggtggtggcgccctcgctggtggacgcctacattaacattggggcgcgctggtcgctggaccccatggacaacgtaaatcccttcaaccaccaccgcaatgcgggcttgcgctaccgctccatgctcctgggcaacgggcgctacgtgccattccacatccaggtgccccagaaattttttgccattaagagcctcctgctcctgcccgggtcctacacctacgagtggaacttccgcaaggacgtcaacatgatcctgcagagttcccttggcaacgacctgcgcacagacggggcctccatcaccttcaccagcattaacctctacgccaccttcttccccatggcgcacaacaccgcctccacgcttgaggccatgctgcgcaacgacaccaatgaccaatccttcaacgactacctctcggcggccaacatgctctatcccatcccggccaacgccaccaacgtgcccatctccatcccctcgcgcaactgggccgcctttcgcggctggtccttcacgcgtctcaagaccaaagagacgccctcgctgggctccgggttcgacccctacttcgtctactcgggctccatcccctacctcgacggcaccttctacctcaaccacaccttcaagaaggtctccatcaccttcgactcttccgtcagctggcccggcaacgaccggctcctgacgcccaacgagttcgaaatcaagcgcaccgtcgacggcgagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggttaactacaaggactaccaggccgtcaccctggcctaccaacacaacaactcgggcttcgttggatacctcgcgcccactatgcgccagggccagccctaccccgccaactacccctacccgctcatcggcaagagcgccgttaccagcgtcacccagaaaaagttcatctgcgacagggtcatgtggcgcatccccttctccagcaacttcatgtccatgggcgcgctcaccgacctcggccagaacatgctctatgctaactccgcccacgcgctagacatgaatttcgaagtcgaccccatggatgagtccacccttctctatgttgtcttcgaagtcttcgacgtcgtccgagtgcaccagccccaccgcggcgtcattgaggccgtctacctgcgcacccccttctcagccggtaacgccaccacataaattcttgcttcttgcaagaagccatggccgcgggctccggcgagcaggagctcagggccatcatccgcgacctggggtgcgggccctacttcctgggcaccttcgataagcgattcccgggattcatggccccgcacaaggtggcctgcgccatcgtcaacacggccggccgcgagaccgggggcgagcattggctggccttcgcctggaacccgcgctcgaacacctgctacctcttcgaccccttcgggttctcggaccagcgcctcaagcaaatctaccagttcgagtacgagggactgctgcgccgcagcgccctggccaccaaggaccgctgcgttaccctggaaaagtccacccagaccgtgcagggtccgcgttcggccgcctgcgggcttttctgctgcatgttcctacacgccttcgtgcactggcccaaccgccccatggacaaaaatcccaccatgaacttgctgacgggggtgcccaacggcatgctccagtcgccccaggtggaacctaccctgcgccgcaaccaggaggcactctaccgcttcctcaactcccactctgcatactttcgctctcaccgcgcgcgcattgagaaggccaccgccttcgaccgcatgaatcaagacatgtaacagtgtgttttaaaatatgtttaataaacagcactttttatgtgacacatgcatttgagataattttattcttaaaaatcgaaggggttctgccgggaggtttcggcatggcccgcgggcagggacacgttgcggaactggtacttggccagccacttgaactcggggatcagcagtttcggcagcagggtgtcggggaacgagtcggtccacagcttccgcgtcagttgcagggcgcccagcaggtcgggcgcggagatcttgaaatcgcagttgggacccgcgttttgcgcgcgagagttgcggtacacagggttgcagcactggaacaccatcagggccggatgcttcacgctcgccagcaccgtagcgtcggtgatcccgtccacgtcgaggtcttcggcgttggccatcccgaagggggtcatcttgcaggtctgccggcccatggtgggcacgcagccgggcttgtggttgcaatcgcagtgcagggggatcagcatcatctgggcctggtcggcgttcatccccgggtacatggccttcatgaaagcctccagctgcttaaacgcctgctgggccttggctccctcggtgaagaagaccccgcaggacttgctagaaaactggttggtagcgcacccggcgtcgtgcacgcagcagcgcgcgtcgttgttggccagctgcaccacgctgcgcccccagcggttctgggtaatcttggcccggtcggggttctcctttagcgcgcgttgcccgttctcgcttgccacatccatctcgatcatgtgctccttctggatcatggtggtcccgtgcaggcaccgcagcttgccctcgacttcggtacagccgtgcagccacagcgcgcaccccgtgctctcccagttcttgtgggcgatctgggaatgcgcatgcacgaacccctgcaggaagcggcccatcatggtcgtcagggtcttgttactggtaaaggtcagcggaatgccgcggtgctcctcgttgatgtacaggtggcagatgcggcgatacacctcgccctgctcgggcatcagttggaagttggattttaggtcgctttccacacggtagcgctccatcagcatattcatgatttccatgcccttctcccaggccgatacaatgggcaggctcagggggttcgtcaccgccatcttagcgctagcagccttcgtcagcgggtcgttctcattgagagtctcaaagctccgcttgccgtccttctcggtgatccgcacgggggggtagctgaagcccacggccgccagctcctcctcggcctctctttcgtcctcgctgtcctggctgacgtcctgcaggggcacatgcttcgttttgcggggtttctttttgggcggctgctgcggcggcggtggttgttcctgaggcgagggggagcgcgagttctcgctcaccactactatctcttcttcttggtccgaggccacgcggcggtaggtatgtctcttcaggggcagaggcggaggcgacgggctctcgcggcccggcgggtggctggcagagccccttccgcgatcgggggtgcgctcccggcggcgctctaactgacttcctccgcggccggccattgtgttctcctagggaacaacaacaagcatggagactcagccatcgtcgccaacctcgccatctgcccccaccgccgacaagaagcagcagcagaatgagagcttaaccgccccgccgcccagccccgccacctttgtcgcggccccagacatgcaagagatggaggaatccattcagattgacctgggctatgtgacgcccgcggagcacgaggaggagcttgcagtgcgcttttcaacccaggaagagatacaccaagaacagccagagcaggaagcaaagagcgagcatgactacctccaccagagcgggggggaggacgccctcatcaagcatctggcccggcaggccatcatcgtcaaggacgcgctgcttgaccgcaccgaggtgcccctcagcgtggaggagctcagccgcgcctacgagctcaacctcttctcgccgcgcgtgccccccaagcgccagcccaacggcacctgcgagcccaacccacgcctcaacttctacccggtcttcgcggtgcccgaggccctggccacctaccacatctttttcaagaaccaaaggatccctgtctcctgtcgcgccaaccgcacccgcgccgactcccttttcaacctgggccccggtgcccgcctacctgatatcgcctccttggaagaggttcccaagatcttcgagggtctgggcagcgacgagactcgggccgcaaacgctctgcaaggagaaggaggagatcatgagcaccacagcgccctggtggagttggaaggcgacaacgcgcgtctggcggtgctcaagcgcacgatcgagctgacccatttcgcctacccggcgcttaacctgccccccaaagtcatgagcacggttatggatcaggtgctcatcaagcgcgcgtcgcccatctccaaggagatgcaagaccccgagagctccgaggagggcaagcccgtggtcagcgacgagcagctggcgcggtggctgggaccccaagctagtccccagagcttggaagagcggcgcaagctcataatggccgtggtcctggtgaccgcggagctggagtgtctgcgccgcttcttcgccgacgcagaaattctgcgcaaggtcgaggagaacctgcactacatcttcaggcacgggttcgtacgccaggcctgcaagatctccaacgtggagctgaccaacctggtctcctacatgggcatcttgcacgagaaccgcctggggcagaacgtgctgcacaccaccctgcgcggggaggcccgccgcgactacatccgcgactgcgtttacctctacctctgccacacctggcagacagccatgggcgtgtggcagcagtgtctggaggagcagaacctaaaagagctctgcaagctcctgcagaagaacctcaaggccctgtggaccgggttcgacgagcgcaccaccgcctcggacctggcagacctcattttccccgagcgtctcaggctgacgctgcgcaacggtttgcccgactttatgagtcaaagcatgttgcaaaactttcgctctttcatcctcgaacgctccgggatcctgccggccacctgctccgcgctgccctcggacttcgtgccgctgaccttccgcgagtgccccccgccgctgtggagccactgctacctgctgcgcttggccaactacctggcctaccactcggacgtgatcgaggacgtcagcagcgagggcctgctcgagtgccactgccgctgcaacctctgcacgccgcaccgctccctggcctgcaacccccagctgctgagcgagacccagatcatcggcaccttcgagttgcaagggcccggcgatgagggttctgccgccaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggcgatcctggcccaattgcaagctatccagaaatcccgccaagaattcttgctgaaaaagggccgcggggtctaccttgatccccagaccggtgaggagcttaaccccggcttcccccaggatgccccgaggaagcagcaagaagctgaaagtggagctgccgcccgtggaggatttggaggaagactgggagagcagtcaggcagaggaggaggagatggaagactgggacagcactcaggcagaggacagcctgcaagacagtctggaagacgaggaggaggcagaggaggtggaagaagtagccgccgccgccagaccgtcgtcctcggcggagaaagcaagcagcacggataccatctccgctccgggtcggggtcccgctcgaccccacagtagatgggacgagaccgggcgattcccgaaccccaccacccagaccggtaagaaggagcggcagggatacaagtcctggcgggggcacaaaaacgccatcgtctcctgcttgcaagcttgcgggggcaacatctcattcacccggcgctacctgctctttcaccgcggggtgaacttcccccgcaacatcttgcattactaccgtcacctccacagcccctactacttccaagaagaggcagaaaaagacaaaaccagcagctagaaaatccacagcggcggcggcggcaggtggactgaggatcgcggcgaacgagccggcgcagacccgggaactgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcacgcttgaggacgccgaggctctcttcaacaagtactgcgcactcactcttaaagagtagcccgcgcccgcccacacacggaaaaaggcgggaattacgtcacctgtgcacccccacccagcaccgctatgagcaaagaaattcccacgccttacatgtggagctaccagccccagatgggcctggccgccggcgccgcccaggactactccacccgcatgaattggctcagcgccgggcccgggatgatctcacgggtgaatgacatccgcgcccaccgaaaccagatactcctagaacagtcagcgctcaccgccacgccccgcaatcacctcaatccgcgtaattggcccgccgccctagtgtaccaggaaattccccagcccacgaccgtactacttccgcgagacgcccaggccgaagtccagctgactaactcaggtgtccagctggcgggcggcgccaccctgtgtcgtcaccaccccgctcagggtataaagcggctggtgatccggggcagaggcacacagctcaacgacgaggtggtgagctcttcactgggtttgcgacctgacggagtcttccaactcgccggatcgggaagatcttttcggggcaacatctcattcacccggcgctacctgctctttcaccgcggggtgaacttcccccgcaacatcttgcattactaccgtcacctccacagcccctactacttccaagaagaggcagaaaaagacaaaaccagcagctagaaaatccacagcggcggcggcggcaggtggactgaggatcgcggcgaacgagccggcgcagacccgggaactgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcacgcttgaggacgccgaggctctcttcaacaagtactgcgcactcactcttaaagagtagcccgcgcccgcccacacacggaaaaaggcgggaattacgtcacctgtgcacccccacccagcaccgctatgagcaaagaaattcccacgccttacatgtggagctaccagccccagatgggcctggccgccggcgccgcccaggactactccacccgcatgaattggctcagcgccgggcccgggatgatctcacgggtgaatgacatccgcgcccaccgaaaccagatactcctagaacagtcagcgctcaccgccacgccccgcaatcacctcaatccgcgtaattggcccgccgccctagtgtaccaggaaattccccagcccacgaccgtactacttccgcgagacgcccaggccgaagtccagctgactaactcaggtgtccagctggcgggcggcgccaccctgtgtcgtcaccaccccgctcagggtataaagcggctggtgatccggggcagaggcacacagctcaacgacgaggtggtgagctcttcactgggtttgcgacctgacggagtcttccaactcgccggatcgggaagatcttccttcacgcctcgtcaggccgtgctgactttggagagttcttcctcgcaacctcgctcgggcggcatcggcactctccagtttgtggaggagttcactccctcggtctacttcaaccccttctccggctcccccggccactatccggacgagttcatcccgaacttcgatgccatcagcgaatcggtagacggctacgattgaatgtcccatggtggcgcggctgacctagctcggcttcgacacctggaccactgccgccgctttcgctgcttcgctcgggacctcgccgagtttacctactttgagctgtccgaggagcaccctcagggcccggcccacggagtgcggatcgtcgtcgaagggggcctagactcccacctgcttcgtatcttcagccagcgcccgatcctggtccagcgccaacagggcaacaccctcctgaccctttactgcatctgcaaccaccccggcctgcacgaaagtctttgttgtctgctgtgtactgagtataataaaagctgagatcagcgactactccggactcgattgtgttccagcagtctggcgataccaagggttgcatccactgctcctgcgactcccccgagtgcgttcacaccctcatcaagaccctatgcggcctccgcgacctcctccccatgaactaatcaactaaccccttaccccattacccatccagtaaaaaaaataaagattaaagagacgatgattttgaattactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcactgtcttccggatcgctgtccaggagcgccagctgttgggctcgcggttgagaaggaactcttcgcggtccttccagtactcttcaagggggaacccgtcctggtcggcacgggactccgcgcaaggacctaagcgtctccagatccacgggatctgaaaaccgttgaacgaaggcttcgagccagtcgcagtcgcaagtctagagccaccatgttcgtcttcctggtcctgctgcccctggtctcatctcagtgcgtgaatctgactacaagaactcagctgcctcccgcctacaccaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaagatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagagatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgcctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacaaggtggaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgattccgaacccgtgctgaagggagtgaagctgcattacacctgaggatccctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgatcaataaagaatcacttacttgaaatctgaaaccaggtctctgtccatgttttctgtcagcagcacttcgctcccctcttcccagctctggtactgcaggccccggcgggctgcaaacttcctccacactctgaaggggatgtcaaattcctcctgtccctcaatcttcattttttatttctattagatgtccaaaaagcgcgcgcgggtggatgatggcttcgaccccgtgtatccctacgatgcagacaacgcaccgaccgtgcccttcatcaaccctcccttcgtctcttcagatggattccaagaaaagcccctgggggtgttgtcccttaggctggccgaccctgtcaccaccaagaatggggaaattaccctcaagctgggggagggggtggaccttgacgactcgggaaaactcattgcaaacacagtaaacaaggccattgcccctctcagtttttccaacaacaccatttcccttaacatggatacccctttatacaccaaagatggaaaactatccttacaagtttctccaccattaagtatattaaaatcaacaattttgaatacattagctctagcttttggctcaggtttaggactcagtggcagcgccctggcagtacagttagcctctccacttacatttgatgataaagggaatataaagattaccctaaacaggggattgcatgttacaacaggagatgcaattgaaagcaacatcagttgggctaaaggtataaaatttgaagatggtgccatagctacaaacattggtaaggggctagagttcggaaccagtagtacagaaacaggagttaataatgcttatccaatccaagttaaacttggctctggtctcagctttgacagcacaggagccataatggctggcaataaagactatgataaattaactttgtggacaacgcctgacccatcaccaaactgtcaaatacttgcagaaaatgatgcaaaactaacactttgcttaactaagtgtgacagtcaaatactggccactgtatcagttttggttgttagaagtggaaacttaaacccaattactggcacagtaagcagtgctcaagtttttctacgttttgatgcaaatggtgttcttttaacagaacactctacactaaaaaaatactggggctacaagcaaggagatagcatagatggcactccatacaccaatgctgttggttttatgccaaattcaacagcttatccaaagacccaaagttctactactaaaaataatatagtgggtcaagtatacatgaatggagatgtttcaaaacccatgcttcttactataactcttaatggtactgatgacaccaccagtgcatactcaatgtcattttcatacacctggactaacggaagctatatcggagcaacatttggagctaactcatacaccttctcctacatagcccaacaataatcccaccctgcatgccaacccaccttttccctctatttataaatggaaactgaaacaaaaataaagttcaagtgttttattgattcaacagtttttcacaggattcgagtagttattttccctccaccctcccatctcatggaatacactatcctctccccacgcacagccttaaacatctgaatgctattggtaatggacatggttttgatctccacattccacacagtttcagagcgagacagtctcgggtcggtcaaggagatgaaaccctccgggcactcctgcatctgcacctcacagttcaacagctgagggctgtcctcggtgattggaatcacagttatctggaataagagcgatgagaatcataatccgcaaacgggatcgggcggttgtggcgcatcaggccccgcagcagtcgctgtctgcgccgctccgtcaagctgctactcaaggggtccgggtccagggactccctgcgcatgatgccaatggccctgagcatcagtcgcctggtacggcgggcgcagcagcggatgcggatctcactcaggtcggagcagtacgtgcagcacagcaccaccaagttgttcaacagtccatagttcaacgtgctccagccaaaactcatttgtggaactatgctgcccacatgtccatcgtaccagatcctgatgtaaatcaggtggcgtcccctccagaacacactgcccatgtacatgatctccttgggcatgtgcaggttcaccacctcccggtaccacatcacccgctggttgaacatgcagccctggataattctgcggaaccagatggcaagtaccgtcccgcccgccatgcagcgcagggaccccgggttctggcaatggcagtggatcacccaccgctcgcgaccgtggatcaactgggaactaaacaagtctatgttggcacagcacaggcacacgctcatgcatgtcttcagcactctcaattcctcgggggtcaggaccatatcccagggcacagggaactcttgcaggacagtgaacccggccgaacagggcaatcctcgcacggaacttacattgtgcatggacagggtatcgcaatcaggcagcaccggatgatcctccaccagagaagcgcggctctcggtctcctcacagcgaggtaaggtggccggcggttggtacggatgatggcgagataacgctaatcgtgttctggatcgtgtcatgatggagctgtttccggacattttcgtatttcacaaagcagaacctggtccgggcactgcacaccgctcgtcggcgacggtctcggcgcttcgagcgctcaatgttgaagttatagaacagccactccctcagaacgtgcagtatctcctgagcctcttgggtgatgaaaatcccatccgccctgatggctctgattacatcaaccacggtggaatgggccaaacccagccagatgatgcaattttgttgggtttcggtgacggcgggggagggaagaacaggaagaaccatgattaactttattccaaacggtctcggaacacttcaaaatgcaggtcccggaggtggcacctctcgcccccactgtgttggtggaaaataacagccaggtcaaaggtaacacggttctcgagatgttccacggtggcttccagcaaagcctccacgcgcacatccagaaacaagaggacagcgaaagcgggagcgttttctaattcctcaatcatcatattacactcctgcaccatgcctagataattttcatttttccagccttgaatgattcgtattagttcctgaggtaaatccaagccagccatgataaaaagctcgcgcagagcgccctccaccggcattcttaagcacaccctcataattccaacagattctgctcctggttcacctgtagtagattaacaagtggaatatcaattgctctgccgcaatccctaagctcctcccttagcagtaactgtatgtactcattcatatcttctccgaaatttttagccataggaccaccaggaacaagagaagggcaagccacattacagataaagcgaagtcctccccagtgagcattgccaaatgtaagattgaaataagcatgctggctagacccggtgatatcttccagataactggacagaaaatcaggcaagcaatttttaagaaaattaacaaaagaaaagtcgtctaggtgcacgtttagagcctcaggaacaacgatggaataagtgcaaggagtacgttccagcatggttagtgtttttggtgatctgtagaacaaaaaataaacatgcaatattaaaccatgctagcctggcgaacaggtggataaatcactctttccaacaccaggcaggctacagggtctccggcgcgaccattgtagaagctgacattatgattaaaaagcatcaccgacagaccttcccggtggccggcatggatgattcgagaagaagcatacactccgggaacattggcgtccgtgagtgaaaaaaagcgacctataaagccttgaggcactacaatgcttaatcttaattccagcaaagcgaccccatgcggatgaagcacaaaattggcaggtgcgtaaaaaatgtaattactccccttctgcacaggcagcaaagcccccgctccctccagaaacacatacaaaacctgagcgtccatagcttaccgagcacggcaggcgcaagagtcagagaaaaagctgagctctaacctaactgcccgcttctgtactcaatatatagccctaacctcactgacgtaaaggccaaggtctaaaaatacccgccaacacgcccagaaaccggtgacacactaaaaaaatacgtgcacttcctcaaacgcccaaactggcgtcatttccggtttcccacgctacgtcacctctcaacgactttcaaattccgtcgaccgttaaacacatcagttaccccgcccctaacgaacgccgctgtcacagccaatcagcgcgccccatccccaaattttcacgccttatttgcatattaactcacacaaaaaaaataaggtatattattgatgatgaagcttttaat

SEQ ID NO: 2 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1273 aa, Artificial Sequence
  Synthetic protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1249 aa, Artificial Sequence
  Synthetic protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCS

SEQ ID NO: 5 - protein, 1268 aa, Artificial Sequence
  Synthetic protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGV

SEQ ID NO: 6 - DNA, 3822 bp, SARS-CoV-2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 7 - protein, 1270 aa, Artificial Sequence
  Synthetic protein
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1273 aa, Artificial Sequence
  Synthetic protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1273 aa, Artificial Sequence
  Synthetic protein
MFVFLVLLPLVSSQCVNLTTTTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPHGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTDWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1273 aa, Artificial Sequence
  Synthetic protein
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1271 aa, Artificial Sequence
  Synthetic protein
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1270 aa, Artificial Sequence
  Synthetic protein
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVEPERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 3813 bp, Artificial Sequence
  Synthetic nucleic acid
atgttcgtgtttctggtgctgctgcctctggtgagctcccagtgcgtgaacttcaccacaagaacccagctgccccctgcctacaccaattccttcacaaggggcgtgtactatcccgacaaggtgtttcgctctagcgtgctgcactccacacaggatctgtttctgcctttcttttctaacgtgacctggttccacgccatccacgtgagcggcaccaatggcacaaagcggttcgccaatccagtgctgccctttaacgacggcgtgtacttcgcctccaccgagaagtctaacatcatcagaggctggatctttggcaccacactggatagcaagacacagtccctgctgatcgtgaacaatgccaccaacgtggtcatcaaggtgtgcgagttccagttttgtaatgacccattcctgggcgtgtactatcacaagaacaataagtcttggatggagagcgagtttagggtgtactcctctgccaacaattgcacatttgagtacgtgagccagcccttcctgatggacctggagggcaagcagggcaatttcaagaacctgcgcgagttcgtgtttaagaatatcgatggctacttcaagatctactccaagcacaccccaatcaacctggtgaggggactgccacagggcttctctgccctggagccactggtggacctgcccatcggcatcaacatcacccgctttcagacactgcacatcagctacctgacaccaggcgatagctcctctggatggaccgcaggagcagcagcctactatgtgggctacctgcagcccaggaccttcctgctgaagtataacgagaatggcaccatcacagacgcagtggattgcgccctggaccccctgtctgagaccaagtgtacactgaagagctttaccgtggagaagggcatctaccagacaagcaatttccgggtgcagcctaccgagtccatcgtgagatttcccaatatcacaaacctgtgcccttttggcgaggtgttcaacgccacccgcttcgccagcgtgtatgcctggaataggaagcgcatctccaactgcgtggccgactattctgtgctgtacaacagcgcctccttctctacctttaagtgctacggcgtgagccccacaaagctgaatgacctgtgctttaccaacgtgtatgccgattccttcgtgatcaggggcgacgaggtgcgccagatcgcaccaggccagacaggcaatatcgccgactacaactataagctgcctgacgatttcaccggctgcgtgatcgcctggaacagcaacaatctggatagcaaagtgggcggcaactacaattatctgtaccggctgtttagaaagtctaacctgaagccattcgagagggacatctccacagagatctaccaggccggctctaccccctgcaatggcgtgaagggctttaactgttatttccctctgcagagctacggcttccagccaacctacggcgtgggctatcagccctaccgcgtggtggtgctgtcttttgagctgctgcacgcacctgcaacagtgtgcggcccaaagaagagcaccaatctggtgaagaacaagtgcgtgaacttcaacttcaacggactgaccggcacaggcgtgctgaccgagtccaacaagaagttcctgccttttcagcagttcggccgggacatcgccgataccacagacgccgtgagagaccctcagaccctggagatcctggatatcacaccatgctccttcggcggcgtgtctgtgatcacaccaggcaccaatacaagcaaccaggtggccgtgctgtaccagggcgtgaattgtaccgaggtgcccgtggcaatccacgcagaccagctgacccctacatggagggtgtattctaccggcagcaacgtgttccagacacgcgccggatgcctgatcggagcagagcacgtgaacaatagctacgagtgcgatatccctatcggcgccggcatctgtgcctcctatcagacccagacaaactccccacggagagcccggtctgtggcaagccagtccatcatcgcctacaccatgagcctgggcgtggagaacagcgtggcctattccaacaattctatcgccatccctaccaacttcacaatctccgtgaccacagagatcctgccagtgagcatgaccaagacatccgtggactgcacaatgtacatctgtggcgattccaccgagtgctctaacctgctgctgcagtatggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtacggcgattgcctgggcgacatcgcagccagagacctgatctgtgcccagaagtttaatggcctgaccgtgctgcctccactgctgacagatgagatgatcgcccagtatacatctgccctgctggcaggaaccatcacaagcggatggaccttcggcgcaggagccgccctgcagatcccctttgccatgcagatggcctacaggttcaacggcatcggcgtgacccagaatgtgctgtatgagaaccagaagctgatcgccaatcagtttaactccgccatcggcaagatccaggactctctgagctccacagcaagcgccctgggcaagctgcaggatgtggtgaatcagaacgcccaggccctgaataccctggtgaagcagctgtctagcaacttcggcgccatctcctctgtgctgaatgatatcctgagccggctggaccctcctgaggcagaggtgcagatcgaccggctgatcacaggcagactgcagtccctgcagacctacgtgacacagcagctgatcagggcagcagagatcagggcatctgccaatctggccgccaccaagatgagcgagtgcgtgctgggccagtccaagagagtggacttttgtggcaagggctaccacctgatgagcttcccacagtccgcccctcacggcgtggtgtttctgcacgtgacctatgtgccagcccaggagaagaacttcaccacagcaccagccatctgccacgatggcaaggcacactttcctcgggagggcgtgttcgtgagcaacggcacccactggtttgtgacacagagaaatttctacgagccacagatcatcaccacagacaataccttcgtgagcggcaactgtgacgtggtcatcggaatcgtgaacaataccgtgtacgatcctctgcagccagagctggactcttttaaggaggagctggataagtatttcaagaatcacaccagccccgacgtggatctgggcgacatctctggcatcaatgccagcgtggtgaacatccagaaggagatcgaccgcctgaacgaggtggccaagaatctgaacgagtccctgatcgatctgcaggagctgggcaagtatgagcagtacatcaagtggccctggtacatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactatacctga

SEQ ID NO: 14 - DNA, 3822 bp, Artificial Sequence
  Synthetic nucleic acid
atgttcgtgtttctggtgctgctgcctctggtgagctcccagtgcgtgaacctgaccacaaggacccagctgccccctgcctacaccaattccttcacacggggcgtgtactatcccgacaaggtgtttagatctagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtactcctctgccaacaattgcacatttgagtacgtgagccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctacctgcagcccaggaccttcctgctgaagtataacgagaatggcaccatcacagacgcagtggattgcgccctggaccccctgtctgagaccaagtgtacactgaagagctttaccgtggagaagggcatctaccagacaagcaatttcagggtgcagcctaccgagtccatcgtgcgctttcccaatatcacaaacctgtgcccttttggcgaggtgttcaacgccacccgcttcgccagcgtgtatgcctggaataggaagcgcatctccaactgcgtggccgactattctgtgctgtacaacagcgcctccttctctacctttaagtgctacggcgtgagccccacaaagctgaatgacctgtgctttaccaacgtgtatgccgattccttcgtgatcaggggcgacgaggtgcgccagatcgcaccaggccagacaggcaatatcgccgactacaactataagctgcctgacgatttcaccggctgcgtgatcgcctggaacagcaacaatctggatagcaaagtgggcggcaactacaattatctgtaccggctgtttagaaagtctaacctgaagccattcgagagggacatctccacagagatctaccaggccggctctaccccctgcaatggcgtgaagggctttaactgttatttccctctgcagagctacggcttccagccaacctacggcgtgggctatcagccctaccgcgtggtggtgctgtcttttgagctgctgcacgcacctgcaacagtgtgcggcccaaagaagagcaccaatctggtgaagaacaagtgcgtgaacttcaacttcaacggactgaccggcacaggcgtgctgaccgagtccaacaagaagttcctgccttttcagcagttcggcagggacatcgcagataccacagacgccgtgcgcgaccctcagaccctggagatcctggatatcacaccatgctccttcggcggcgtgtctgtgatcacaccaggcaccaatacaagcaaccaggtggccgtgctgtaccagggcgtgaattgtaccgaggtgcccgtggcaatccacgcagaccagctgacccctacatggcgggtgtattctaccggcagcaacgtgttccagacaagagccggatgcctgatcggagcagagcacgtgaacaatagctacgagtgcgatatccctatcggcgccggcatctgtgcctcctatcagacccagacaaactccccacggagagcccggtctgtggcaagccagtccatcatcgcctacaccatgagcctgggcgccgagaacagcgtggcctattccaacaattctatcgccatccctaccaacttcacaatctccgtgaccacagagatcctgccagtgagcatgaccaagacatccgtggactgcacaatgtacatctgtggcgattccaccgagtgctctaacctgctgctgcagtatggctctttttgtacccagctgaatagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtacggcgattgcctgggcgacatcgcagccagagacctgatctgtgcccagaagtttaatggcctgaccgtgctgcctccactgctgacagatgagatgatcgcccagtatacatctgccctgctggcaggaaccatcacaagcggatggaccttcggcgcaggagccgccctgcagatcccctttgccatgcagatggcctacagattcaacggcatcggcgtgacccagaatgtgctgtatgagaaccagaagctgatcgccaatcagtttaactccgccatcggcaagatccaggactctctgagctccacagcaagcgccctgggcaagctgcaggatgtggtgaatcagaacgcccaggccctgaataccctggtgaagcagctgtctagcaacttcggcgccatctcctctgtgctgaatgatatcctgagccggctggacccaccagaggcagaggtgcagatcgaccggctgatcacaggcagactgcagtccctgcagacctacgtgacacagcagctgatcagggcagcagagatcagggcatctgccaatctggccgccaccaagatgagcgagtgcgtgctgggccagtccaagagagtggacttttgtggcaagggctaccacctgatgagcttcccacagtccgcccctcacggcgtggtgtttctgcacgtgacctatgtgccagcccaggagaagaacttcaccacagcaccagccatctgccacgatggcaaggcacactttccccgggagggcgtgttcgtgagcaacggaacccactggtttgtgacacagcgcaatttctacgagccacagatcatcaccacagacaatacattcgtgtccggcaactgtgacgtggtcatcggaatcgtgaacaataccgtgtacgatcctctgcagccagagctggactcttttaaggaggagctggataagtatttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtacatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactatacctga

SEQ ID NO: 15 - DNA, 3822 bp, Artificial Sequence
  Synthetic nucleic acid
atgttcgtgtttctggtgctgctgcctctggtgagctcccagtgcgtgaacctgaccacaaccacacagctgccccctgcctataccaattccttcacacgcggcgtgtactatcctgacaaggtgtttcggtctagcgtgctgcactccacacaggatctgtttctgccattcttttctaacgtgacctggttccacgccatccacgtgagcggcaccaatggcacaaagcggttcgacaatccagtgctgccctttaacgatggcgtgtacttcgcctccaccgagaagtctaacatcatccggggctggatctttggcaccacactggacagcaagacacagtccctgctgatcgtgaacaatgccaccaacgtggtcatcaaggtgtgcgagttccagttttgtaatgatcccttcctggacgtgtactatcacaagaacaataagtcttggatgaagagcgagtttagagtgtattcctctgccaacaattgcacatttgagtacgtgtcccagcctttcctgatggacctggagggcaagcagggcaatttcaagaacctgagagagttcgtgtttaagaatatcgatggctacttcaagatctactccaagcacaccccaatcaacctggtgagggacctgccacacggcttctctgccctggagccactggtggatctgcccatcggcatcaacatcaccagatttcagacactgctggccctgcacaggagctacctgacacccggcgacagctcctctggatggaccgccggcgctgccgcctactatgtgggctatctgcagcctcgcaccttcctgctgaagtacaacgagaatggcaccatcacagacgcagtggattgcgccctggaccccctgtctgagaccaagtgtacactgaagagctttaccgtggagaagggcatctatcagacaagcaatttccgcgtgcagccaaccgagtccatcgtgcggtttcccaatatcacaaacctgtgcccttttggcgaggtgttcaacgcaaccaggttcgcaagcgtgtacgcatggaatcgcaagcggatctccaactgcgtggccgactattctgtgctgtacaacagcgcctccttctctacctttaagtgctatggcgtgagcccaacaaagctgaatgacctgtgctttaccaacgtgtacgccgattccttcgtgatccggggcgacgaggtgcggcagatcgcaccaggacagacaggcaagatcgcagactacaattataagctgcctgacgatttcaccggctgcgtgatcgcctggaactctaacaatctggatagcaaagtgggcggcaactacaattatagatacaggctgtttagaaagtctaatctgaagccattcgagagggacatctccacagagatctaccaggccggctctaccccctgcaatggcgtgcagggctttaactgttatttccctctgcagagctacggcttccagccaaccaacggcgtgggctatcagccctaccgggtggtggtgctgtcttttgagctgctgcacgcacctgcaacagtgtgcggaccaaagaagagcaccaatctggtgaagaacaagtgcgtgaacttcaacttcaacggactgaccggaacaggcgtgctgaccgagtccaacaagaagttcctgccatttcagcagttcggcagagacatcgccgataccacagacgccgtgagggaccctcagaccctggagatcctggatatcacaccatgctccttcggcggcgtgtctgtgatcacacccggcaccaatacaagcaaccaggtggccgtgctgtatcagggcgtgaattgtaccgaggtgccagtggcaatccacgcagaccagctgacccctacatggcgcgtgtactctaccggcagcaacgtgttccagacaagggcaggatgcctgatcggagcagagcacgtgaacaatagctatgagtgcgatatccccatcggcgccggcatctgtgcctcctaccagacccagacaaactcccggagaagggccagatctgtggccagccagtccatcatcgcctataccatgagcctgggcgccgagaacagcgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagggccctgacaggaatcgcagtggagcaggacaagaacacacaggaggtgttcgcccaggtgaagcagatctacaagaccccacccatcaaggactttggcggcttcaacttcagccagatcctgcccgatcctagcaagccctccaagcggagcttcatcgaggacctgctgttcaacaaggtgaccctggccgatgccggcttcatcaagcagtatggcgattgcctgggcgacatcgcagcaagggacctgatctgtgcccagaagtttaatggcctgaccgtgctgcctccactgctgacagatgagatgatcgcccagtacacatctgccctgctggcaggaaccatcacaagcggatggaccttcggcgcaggagccgccctgcagatcccttttgccatgcagatggcctatcgcttcaacggcatcggcgtgacccagaatgtgctgtacgagaaccagaagctgatcgccaatcagtttaactccgccatcggcaagatccaggactctctgagctccacagcaagcgccctgggcaagctgcaggatgtggtgaatcagaacgcccaggccctgaataccctggtgaagcagctgtctagcaacttcggcgccatctcctctgtgctgaatgatatcctgagcagactggacccccccgaggccgaggtgcagatcgacagactgatcacaggcaggctgcagtccctgcagacctacgtgacacagcagctgatcagggccgccgagatcagggcctctgccaatctggccgccaccaagatgagcgagtgcgtgctgggccagtccaagagggtggatttttgtggcaagggctatcacctgatgagcttcccacagtccgcccctcacggagtggtgtttctgcacgtgacctacgtgccagcccaggagaagaacttcaccacagcaccagcaatctgccacgacggcaaggcacactttccaagagagggcgtgttcgtgagcaacggcaccgattggtttgtgacacagaggaatttctacgagccccagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgaccggctgaacgaggtggccaagaatctgaacgagtccctgatcgatctgcaggagctgggcaagtatgagcagtacatcaagtggccttggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagccagtgctgaagggcgtgaagctgcactacacctga

SEQ ID NO: 16 - DNA, 3822 bp, Artificial Sequence
  Synthetic nucleic acid
atgttcgtgtttctggtgctgctgcctctggtgagctcccagtgcgtgaatttcaccaacagaacacagctgccttctgcctacaccaatagcttcacacggggcgtgtactatccagacaaggtgtttagatctagcgtgctgcacagcacacaggatctgtttctgccattcttttccaacgtgacctggttccacgccatccacgtgtccggcaccaatggcacaaagcggttcgacaatcccgtgctgccttttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaattatcccttcctgggcgtgtactatcacaagaacaataagtcttggatggagagcgagtttagggtgtactcctctgccaacaattgcacatttgagtatgtgagccagcctttcctgatggacctggagggcaagcagggcaatttcaagaacctgagcgagttcgtgtttaagaatatcgatggctacttcaagatctactccaagcacacccccatcaacctggtgcgcgacctgcctcagggcttctctgccctggagcccctggtggatctgcctatcggcatcaacatcacccggtttcagacactgctggccctgcacagaagctacctgacacccggcgacagctcctctggatggaccgccggcgctgccgcctactatgtgggctacctgcagcctaggaccttcctgctgaagtataacgagaatggcaccatcacagacgcagtggattgcgccctggaccccctgtccgagaccaagtgtacactgaagtcttttaccgtggagaagggcatctaccagacatctaatttcagggtgcagccaaccgagagcatcgtgcgctttcctaatatcacaaacctgtgcccatttggcgaggtgttcaacgccacccgcttcgccagcgtgtatgcctggaataggaagcgcatcagcaactgcgtggccgactattccgtgctgtacaacagcgcctccttctctacctttaagtgttacggcgtgtctcctacaaagctgaatgacctgtgctttaccaacgtgtatgccgatagcttcgtgatcaggggcgacgaggtgcgccagatcgcaccaggacagaccggaacaatcgcagactacaattataagctgcctgacgatttcaccggctgcgtgatcgcctggaactccaacaatctggattctaaagtgggcggcaactacaattatctgtaccggctgtttagaaagtccaacctgaagccattcgagcgggacatcagcacagagatctaccaggcaggctccaccccatgcaatggagtgaagggctttaactgttatttcccactgcagagctacggcttccagcccacatatggcgtgggctatcagccttacagagtggtggtgctgtcctttgagctgctgcacgcaccagcaacagtgtgcggacccaagaagtctaccaatctggtgaagaacaagtgcgtgaacttcaacttcaacggactgaccggaacaggcgtgctgaccgagtccaacaagaagttcctgccatttcagcagttcggcagggacatcgcagataccacagacgccgtgcgcgacccacagaccctggagatcctggatatcacaccctgcagcttcggcggcgtgtccgtgatcacaccaggaaccaatacaagcaaccaggtggccgtgctgtaccagggcgtgaattgtaccgaggtgcctgtggcaatccacgcagaccagctgaccccaacatggcgggtgtattctaccggcagcaacgtgttccagacaagagccggctgcctgatcggcgccgagtatgtgaacaattcttacgagtgcgatatccctatcggcgccggcatctgtgccagctaccagacccagacaaacagcccacggagagcacggtccgtggcaagccagtccatcatcgcctacaccatgtctctgggcgccgagaatagcgtggcctattccaacaattctatcgccatcccaaccaacttcacaatctccgtgaccacagagatcctgcccgtgtctatgaccaagacaagcgtggactgcacaatgtacatctgtggcgattccaccgagtgctctaacctgctgctgcagtatggcagcttttgtacccagctgaatagagccctgacaggcatcgccgtggagcaggacaagaacacacaggaggtgttcgcccaggtgaagcagatctacaagaccccccctatcaaggactttggcggcttcaacttcagccagatcctgcctgatccaagcaagccatccaagaggtcttttatcgaggacctgctgttcaacaaggtgaccctggccgatgccggcttcatcaagcagtacggcgattgcctgggcgacatcgcagcaagggacctgatctgtgcccagaagtttaatggcctgaccgtgctgccacccctgctgacagatgagatgatcgcccagtatacatccgccctgctggccggcaccatcacatctggatggaccttcggcgcaggagccgccctgcagatcccctttgccatgcagatggcctacaggttcaacggcatcggcgtgacccagaatgtgctgtatgagaaccagaagctgatcgccaatcagtttaactccgccatcggcaagatccaggactccctgagctccacagcctctgccctgggcaagctgcaggatgtggtgaatcagaacgcccaggccctgaataccctggtgaagcagctgtctagcaacttcggcgccatctcctctgtgctgaatgatatcctgagccggctggacccccccgaggcagaggtgcagatcgaccggctgatcaccggcagactgcagagcctgcagacctacgtgacacagcagctgatcagggccgccgagatcagggcatccgccaatctggccgccatcaagatgtctgagtgcgtgctgggccagagcaagagagtggacttttgtggcaagggctaccacctgatgagcttccctcagtccgccccacacggagtggtgtttctgcacgtgacctatgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtacgatccactgcagcccgagctggactcctttaaggaggagctggataagtatttcaagaatcacacctctcccgacgtggatctgggcgacatctccggcatcaatgcctctttcgtgaacatccagaaggagatcgaccgcctgaacgaggtggccaagaatctgaacgagtccctgatcgatctgcaggagctgggcaagtatgagcagtacatcaagtggccctggtacatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactatacctga

SEQ ID NO: 17 - DNA, 3816 bp, Artificial Sequence
  Synthetic nucleic acid
atgtttgtgtttctggtgctgctgccactggtgagtagccagtgtgtgaacctgagaacccgaacacagctgcctcctgcctataccaacagcttcaccagaggcgtgtactaccctgacaaggtgttccgatctagcgtgctccatagcacccaggacctgttcttgccttttttctctaacgtgacatggttccacgccattcacgtgtctggcaccaacggaacaaaaagattcgacaaccctgtgctgcccttcaacgacggtgtctattttgccagcaccgagaagagcaacatcatcagaggctggatcttcggaaccaccctggacagcaagacccagagcctgctgatcgtcaataacgcaacaaatgtggtgatcaaggtgtgcgagttccaattttgcaacgatcctttcctggatgtgtactaccacaagaacaacaaaagctggatggaaagtggagtttatagcagcgccaacaactgcaccttcgagtacgtgagccaacctttcctgatggacctcgaagggaaacagggcaacttcaagaaccttagagagttcgtctttaagaacatcgacggctactttaaaatctactccaagcacacccccatcaacctggtgcgggacctgcctcagggctttagcgcgctggaacccttggttgacctgcccatcggcatcaacatcactagattccagacccttctggccctccaccggtcttacctgacacctggcgacagtagttctggctggacagccggcgccgctgcctactacgtgggctatctgcagcctagaaccttcctgctgaagtacaacgagaacggcaccatcaccgacgctgtggattgcgccctggaccctctgtccgaaaccaagtgcacactgaagtccttcaccgtggaaaagggcatctaccagacctctaacttccgggtgcagcctactgaaagcatcgtgcggttcccaaacattacaaacctgtgccctttcggagaagttttcaacgccactcgcttcgcctctgtctatgcctggaacagaaagcggatcagcaattgtgtggccgattacagcgtgctgtacaacagcgccagcttttctacattcaagtgctacggcgtgtctcccaccaagctgaatgatctgtgcttcaccaacgtgtacgccgactcgtttgtgatccggggagacgaagtgcgccagatcgcccctgggcagacaggaaacatcgccgattacaattacaaactgcctgacgattttacaggatgtgtgatagcttggaactccaacaacctcgacagcaaagtgggcggcaactacaattaccggtacagactgtttagaaagagcaacctaaaacccttcgagagagatatctctaccgagatctaccaggccggcagcaagccttgtaatggcgttgagggcttcaactgttacttccctctgcagagctacggcttccagcccaccaacggcgtcgggtaccagccttacagagttgtggttctgagcttcgagctgctccacgctcctgccaccgtgtgtggtcctaagaaaagcaccaacctggtgaagaacaagtgcgtgaatttcaatttcaacggcctgacaggcacaggcgtgctgaccgagagcaacaaaaagttcctgcccttccagcagttcggcagagatattgccgataccacagacgccgtgcgggaccctcaaaccctggaaatcttggacatcacaccttgcagcttcggcggagtgtctgtgatcactcccgggaccaacaccagcaaccaggttgccgtgctgtaccagggcgtcaactgcaccgaagtgccagtggctatacacgccgaccagctgacccctacatggcgggtgtacagcaccggcagcaacgtgttccagaccagagccggctgcctgatcggcgcagagcacgtgaacaactcttatgaatgcgacatccccatcggagccggcatttgcgccagctaccagacacagaccaatagcagaagacgggctagaagcgtggcctcgcagagcataatcgcatacacaatgagcctgggagccgagaacagcgtggcctacagcaacaatagtatcgccatccccacaaattttaccatcagcgtgacaaccgaaatcctgccagtgagcatgacaaagaccagcgtcgactgcacaatgtacatatgtggcgatagcacggagtgcagcaatctgctgctccaatacggcagcttctgcacccagctgaatcgggcactgaccggcatcgccgtggaacaggataaaaatacccaggaggtgtttgcccaggtgaagcagatatataagacccctccgatcaaggacttcggaggcttcaatttcagccagatcctgcccgatccaagcaagcctagcaagcggtccttcatcgaggatctgctgttcaataaggtgaccctggccgacgccggattcatcaaacagtacggcgactgcctgggcgacatcgccgccagagatctgatctgtgctcaaaagttcaacggactgacagtcctgccacctctgttgacagatgaaatgatcgctcagtacacctccgccctcctggccgggacgatcacctctggatggaccttcggcgccggcgctgcactgcagatccctttcgccatgcagatggcctacagattcaacggcatcggagtgacccaaaacgtcctgtacgagaaccagaagctgatcgccaaccagttcaactctgctatcggcaagatccaggacagcctcagcagcaccgccagcgccctgggcaaactccagaacgtggtgaaccagaacgcacaggccctgaataccctggtgaagcagctgagcagcaacttcggcgctatcagctctgtgctgaacgacatcctgagcagactggaccctcccgaggccgaggtgcagattgacaggctgatcacaggcagactgcagtcgctgcaaacttacgtgacccagcaactgatccgggccgccgaaatcagggccagcgccaacctggctgctacaaagatgtccgaatgcgtgttgggccagtccaagagagtggacttctgcggcaagggataccacctgatgagcttccctcagtccgctccccacggcgtcgtgttcctgcatgtgacatacgtgcccgcccaggagaagaatttcaccaccgcccctgccatctgccacgacggcaaggcccacttccccagagagggcgtgttcgtgtccaacggcacccactggttcgtgacccagcggaacttctacgagcctcagatcatcaccaccgataacacattcgtgtccggcaactgcgacgtggttatcggcatcgtgaacaataccgtgtacgaccctctgcagccagaactggattcttttaaggaagagctggacaaatactttaagaaccacacatctcctgatgtggacctgggcgacatcagcggcatcaacgcctccgtggtcaacatccaaaaggagatcgatagactgaacgaggtggccaagaacctcaacgagtctctgattgacctgcaggagctgggcaagtacgagcagtacatcaagtggccttggtacatctggctgggcttcatcgccggcctgatcgctatcgtcatggtgaccatcatgctgtgctgtatgacctcctgctgcagctgtctgaaaggctgctgttcttgcggcagctgttgcaagtttgacgaggacgactccgagcccgtgctgaagggggtgaagctgcactacacgtga

SEQ ID NO: 18 - DNA, 3813 bp, Artificial Sequence
  Synthetic nucleic acid
atgttcgtgttcctggtgctgctgcccctggtgtctagccaatgtgtgaacctgacaacaaggacccagcttcccccagcttacaccaattcatttacaagaggcgtgtattaccccgataaggtgttccgaagcagcgtgctgcacagcacccaggatctcttcctgccttttttcagcaatgtgacttggttccacgtgatcagcggaaccaacggcaccaagcggtttgacaatcctgtgctgcccttcaacgacggcgtgtacttcgccagcatcgagaagagcaacattatccggggctggatcttcggcaccaccctcgatagcaagacccagagcttactgatcgtaaacaacgccaccaatgtcgtaatcaaggtctgtgaatttcagttctgcaacgacccctttctggaccacaagaacaacaagtcgtggatggaaagcgagttcagagtgtacagctccgctaacaattgtacattcgagtacgtgtctcagcctttcctgatggacctggaaggcaagcagggaaacttcaagaatctgagggagttcgtgttcaaaaacatcgacggctacttcaagatctacagcaagcatacccccatcatcgttgaacctgagagagacctgccacagggtttcagcgctctggagcctctggttgacctgcccatcggcatcaacatcacccggtttcagacactgttagccctgcatagatcttacctgaccccaggcgattcttcctctggctggaccgccggagccgcagcctactacgtgggatatctgcagcccagaaccttcctgctgaaatacaacgagaacggaaccatcaccgatgccgtggactgcgccctggaccctctgtctgaaaccaagtgcaccctgaagagcttcaccgtggaaaagggcatctaccagaccagcaactttcgggtgcagcccaccgagagcatcgtgagatttccaaacatcaccaacctgtgtcctttcgacgaggtgtttaatgccacaagattcgccagcgtgtacgcctggaatagaaaaagaatctccaactgcgtggctgattactcagtgctttacaacctggccccattcttcaccttcaagtgctacggcgttagccctaccaagctcaatgatctgtgcttcacgaacgtgtacgccgacagcttcgtgatccggggcgacgaagtcagacagatcgcccctggacagaccggtaatatcgccgactacaattacaagctgcctgatgatttcacaggttgcgtgatcgcctggaactccaacaagctggacagcaaggtgtccggcaactacaactacctgtatagacttttcagaaagtccaacctgaagccattcgagcgggacatcagcactgagatctaccaggccggcaacaaaccctgcaacggagttgccggattcaactgctatttccctctgagatcttactccttcagacctacatacggcgtgggacaccagccttacagagtagtggtgctcagcttcgagcttctgcacgctcctgccaccgtgtgcggccctaagaagagcacgaacctggtgaagaacaaatgtgttaattttaacttcaacggcctgaagggcacaggagtcctgaccgagagcaataaaaaattcttgcccttccagcagttcggaagagacatcgccgacaccacagatgctgtgagagaccctcagaccctggaaatcctcgacatcaccccttgcagcttcggcggcgtcagcgtgatcaccccgggcaccaacacctctaaccaggtggccgtgctgtaccagggcgtgaattgcaccgaggttcctgtggccatccacgcggaccagctgacaccaacatggcgggtgtacagcaccggctccaacgtgtttcagaccagagccggctgtctgatcggcgccgaatatgtgaacaacagctacgaatgcgacatcccaatcggcgccggcatttgcgccagctaccagacacagaccaaaagtcaccggagagctcggagcgtggcctctcagagcattatcgcctataccatgagcctgggggccgagaacagcgtggcctattccaacaacagcatcgccatccctaccaatttcaccatctctgtgaccaccgagatcctgccagtgtccatgacaaagacaagcgtggactgcaccatgtacatctgcggcgactctaccgagtgcagcaacctgctgctgcagtacggcagcttttgcacacagctgaaacgggcgctgacaggaattgccgttgagcaggacaagaacactcaggaggtgtttgcccaagtgaagcagatatataagacccctcctatcaaatacttcggcggctttaacttcagccagatcctccctgatccttctaagcctagcaagcgcagcttcatcgaggacctgctgttcaacaaggtaaccctggctgacgccggcttcatcaagcagtacggtgattgcctgggcgacatcgcagcccgggacctgatctgtgcccaaaaattcaagggcctgactgttctgcctcctctgctgacagatgaaatgatcgcccagtacacctccgccctgctggctggcacaatcaccagcggctggacattcggcgccggcgccgcgctgcagatccctttcgccatgcagatggcctacagattcaacggcatcggagtgactcagaacgtgctgtacgaaaaccagaaactgattgcaaatcagtttaacagcgcaatcggcaagatccaggatagcctgtccagcaccgcctccgctctgggcaagctgcaagacgtggtgaaccacaatgcccaggctctgaacaccttggtgaagcagctgagcagcaagttcggcgccatttcttccgtgctgaacgacatcttcagcagactcgatcctcccgaggccgaggtgcagatcgacagactgatcacgggcagactgcagtctctgcagacatacgtgacacagcaactgatcagagccgctgaaatcagggcctctgccaacctggccgccaccaagatgtctgagtgcgtgctcggccagtctaaaagagtggacttctgcggcaaaggctaccacctgatgagcttcccccagagcgccccccacggcgtggtgttcctacacgttacctacgtgccggctcaagaaaagaactttaccaccgcccctgccatctgccacgacggaaaggcccacttccctcgggagggtgtgtttgtcagcaacggcacacactggttcgtgacacagcggaacttctacgagccccaaatcatcacaacagataacaccttcgtcagcggcaactgtgacgtggtgatcggcatcgtgaacaacaccgtgtatgaccctctgcagcctgagctggacagctttaaggaagagctggacaagtacttcaagaatcacacaagtcctgacgtggatctgggcgatatcagtggcatcaacgcctctgtggtgaacatacaaaaggagatcgacagactgaacgaggtggcaaagaacctgaatgaaagcctgatcgacctgcaagaactgggcaagtacgagcagtacatcaagtggccttggtacatttggctgggatttatcgcaggcctcatcgccatcgtgatggtgacaatcatgctgtgttgcatgaccagctgttgcagctgcctgaaaggctgttgtagctgcggcagctgctgcaagttcgatgaggacgacagcgagcctgtcctgaagggggtgaagctgcactacacatga

SEQ ID NO: 19 - DNA, 3822 bp, Artificial Sequence
  Synthetic nucleic acid
atgttcgtcttcctggtcctgctgcccctggtctcatctcagtgcgtgaatctgactacaagaactcagctgcctcccgcctacaccaattccttcacccggggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaattgcacatttgagtacgtgtcccagcctttcctgatggacctggagggcaagcagggcaatttcaagaacctgagggagttcgtgtttaagaatatcgatggctacttcaagatctactctaagcacacccccatcaacctggtgcgcgacctgcctcagggcttcagcgccctggagccactggtggatctgcctatcggcatcaacatcacccggtttcagacactgctggccctgcacagaagctacctgacacccggcgactcctctagcggatggaccgcaggagcagcagcctactatgtgggctatctgcagcctaggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaaccaggttcgcaagcgtgtacgcatggaataggaagcgcatctctaactgcgtggccgactatagcgtgctgtacaactccgcctctttcagcacctttaagtgctatggcgtgtcccccacaaagctgaatgacctgtgctttaccaacgtgtacgccgattctttcgtgatcaggggcgacgaggtgcgccagatcgcacctggacagacaggcaagatcgccgactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagagatctaccaggccggcagcaccccttgcaatggcgtggagggctttaactgttatttcccactgcagtcctacggcttccagcccacaaacggcgtgggctatcagccttaccgcgtggtggtgctgagctttgagctgctgcacgcaccagcaacagtgtgcggacccaagaagtccaccaatctggtgaagaacaagtgcgtgaacttcaacttcaacggcctgaccggaacaggcgtgctgaccgagtccaacaagaagttcctgccatttcagcagttcggcagggacatcgcagataccacagacgccgtgcgcgacccacagaccctggagatcctggatatcacaccctgctctttcggcggcgtgagcgtgatcacaccaggaaccaatacaagcaaccaggtggccgtgctgtatcaggacgtgaattgtaccgaggtgcctgtggccatccacgccgatcagctgaccccaacatggcgggtgtacagcaccggctccaacgtgttccagacaagagcaggatgcctgatcggagcagagcacgtgaacaattcctatgagtgcgacatcccaatcggcgccggcatctgtgcctcttaccagacccagacaaactctccaaggagagcacggagcgtggcatcccagtctatcatcgcctataccatgtccctgggcgccgagaattctgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctgcagtacggcagcttttgtacccagctgaatagagccctgacaggcatcgccgtggagcaggataagaacacacaggaggtgttcgcccaggtgaagcagatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagcacgggacctgatctgtgcccagaagtttaatggcctgaccgtgctgccacccctgctgacagatgagatgatcgcacagtacacaagcgccctgctggcaggaaccatcacatccggatggaccttcggcgcaggagccgccctgcagatcccctttgccatgcagatggcctataggttcaacggcatcggcgtgacccagaatgtgctgtacgagaaccagaagctgatcgccaatcagtttaactccgccatcggcaagatccaggacagcctgtcctctacagcctccgccctgggcaagctgcaggatgtggtgaatcagaacgcccaggccctgaataccctggtgaagcagctgagctccaacttcggcgccatctctagcgtgctgaatgatatcctgagccggctggacccccccgaggcagaggtgcagatcgaccggctgatcacaggcagactgcagtctctgcagacctatgtgacacagcagctgatcagggcagcagagatcagggcaagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaataccttcgtgagcggcaactgtgacgtggtcatcggcatcgtgaacaataccgtgtatgatccactgcagcccgagctggacagctttaaggaggagctggataagtacttcaagaatcacacctcccctgacgtggatctgggcgacatcagcggcatcaatgcctccgtggtgaacatccagaaggagatcgaccgcctgaacgaggtggccaagaatctgaacgagagcctgatcgatctgcaggagctgggcaagtatgagcagtacatcaagtggccatggtacatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgattccgaacccgtgctgaagggagtgaagctgcattacacctga